SEQUENCE LISTING (138 sequences)

SEQ ID NO: 1 - protein, 16 aa, unidentified
AAVALLPAVLLALLAP

SEQ ID NO: 2 - protein, 11 aa, unidentified
AALLPVLLAAP

SEQ ID NO: 3 - protein, 13 aa, unidentified
GRKKRRQRRRPPQ

SEQ ID NO: 4 - protein, 16 aa, unidentified
  source 1..16: note = Drosophila Antennapedia
RQIKIWFQNRRMKWKK

SEQ ID NO: 5 - RNA, 21 nt, synthetic construct
  source 1..21: mol_type = other RNA
atagacgtgattaatcgcgtt

SEQ ID NO: 6 - RNA, 21 nt, synthetic construct
  source 1..21: mol_type = other RNA
aagatgatccttatcgcgaat

SEQ ID NO: 7 - RNA, 21 nt, synthetic construct
  source 1..21: mol_type = other RNA
ccgcggattatttatcgcgtt

SEQ ID NO: 8 - RNA, 21 nt, synthetic construct
  source 1..21: mol_type = other RNA
aaagtgtcgaattttcgcgat

SEQ ID NO: 9 - RNA, 21 nt, synthetic construct
  source 1..21: mol_type = other RNA
cacctcctaattaatcgcgtt

SEQ ID NO: 10 - RNA, 21 nt, synthetic construct
  source 1..21: mol_type = other RNA
aaagctaggtttatacgacgt

SEQ ID NO: 11 - RNA, 21 nt, synthetic construct
  source 1..21: mol_type = other RNA
gaggactcaatttatcgcgtt

SEQ ID NO: 12 - RNA, 21 nt, synthetic construct
  source 1..21: mol_type = other RNA
aaagacgtactttaatcgcgt

SEQ ID NO: 13 - RNA, 21 nt, synthetic construct
  source 1..21: mol_type = other RNA
tcagttgaccatatcgtacgt

SEQ ID NO: 14 - RNA, 21 nt, synthetic construct
  source 1..21: mol_type = other RNA
tctgtagtggatatacgacgt

SEQ ID NO: 15 - RNA, 21 nt, synthetic construct
  source 1..21: mol_type = other RNA
tagtacgactatttatcgcgt

SEQ ID NO: 16 - RNA, 21 nt, synthetic construct
  source 1..21: mol_type = other RNA
aaagtcgtctttacgtatcgt

SEQ ID NO: 17 - RNA, 21 nt, synthetic construct
  source 1..21: mol_type = other RNA
aacgtacacactaatacgcgt

SEQ ID NO: 18 - RNA, 21 nt, synthetic construct
  source 1..21: mol_type = other RNA
aaccacatccttatacgacgt

SEQ ID NO: 19 - RNA, 21 nt, synthetic construct
  source 1..21: mol_type = other RNA
atgaacgactttttatcgcgt

SEQ ID NO: 20 - RNA, 21 nt, synthetic construct
  source 1..21: mol_type = other RNA
tttcatgcgattacgtatcgt

SEQ ID NO: 21 - RNA, 21 nt, synthetic construct
  source 1..21: mol_type = other RNA
aaacgagtgttttatcgcgat

SEQ ID NO: 22 - RNA, 21 nt, synthetic construct
  source 1..21: mol_type = other RNA
atcgtctagtattttcgcgat

SEQ ID NO: 23 - RNA, 21 nt, synthetic construct
  source 1..21: mol_type = other RNA
tttcatagcgttacgtacgtt

SEQ ID NO: 24 - RNA, 21 nt, synthetic construct
  source 1..21: mol_type = other RNA
acccagatatttttatcgcgt

SEQ ID NO: 25 - RNA, 21 nt, synthetic construct
  source 1..21: mol_type = other RNA
tatcgtctgaatacgtacgtt

SEQ ID NO: 26 - RNA, 21 nt, synthetic construct
  source 1..21: mol_type = other RNA
ttacatccgttttttcgcgat

SEQ ID NO: 27 - RNA, 23 nt, synthetic construct
  source 1..23: mol_type = other RNA
aacgcgattaatcacgtctatct

SEQ ID NO: 28 - RNA, 23 nt, synthetic construct
  source 1..23: mol_type = other RNA
attcgcgataaggatcatcttta

SEQ ID NO: 29 - RNA, 23 nt, synthetic construct
  source 1..23: mol_type = other RNA
aacgcgataaataatccgcggat

SEQ ID NO: 30 - RNA, 23 nt, synthetic construct
  source 1..23: mol_type = other RNA
atcgcgaaaattcgacacttttt

SEQ ID NO: 31 - RNA, 23 nt, synthetic construct
  source 1..23: mol_type = other RNA
aacgcgattaattaggaggtggt

SEQ ID NO: 32 - RNA, 23 nt, synthetic construct
  source 1..23: mol_type = other RNA
acgtcgtataaacctagctttca

SEQ ID NO: 33 - RNA, 23 nt, synthetic construct
  source 1..23: mol_type = other RNA
aacgcgataaattgagtcctcca

SEQ ID NO: 34 - RNA, 23 nt, synthetic construct
  source 1..23: mol_type = other RNA
acgcgattaaagtacgtctttat

SEQ ID NO: 35 - RNA, 23 nt, synthetic construct
  source 1..23: mol_type = other RNA
acgtacgatatggtcaactgaaa

SEQ ID NO: 36 - RNA, 23 nt, synthetic construct
  source 1..23: mol_type = other RNA
acgtcgtatatccactacagatt

SEQ ID NO: 37 - RNA, 23 nt, synthetic construct
  source 1..23: mol_type = other RNA
acgcgataaatagtcgtactagt

SEQ ID NO: 38 - RNA, 23 nt, synthetic construct
  source 1..23: mol_type = other RNA
acgatacgtaaagacgacttttt

SEQ ID NO: 39 - RNA, 23 nt, synthetic construct
  source 1..23: mol_type = other RNA
acgcgtattagtgtgtacgtttt

SEQ ID NO: 40 - RNA, 23 nt, synthetic construct
  source 1..23: mol_type = other RNA
acgtcgtataaggatgtggttaa

SEQ ID NO: 41 - RNA, 23 nt, synthetic construct
  source 1..23: mol_type = other RNA
acgcgataaaaagtcgttcatga

SEQ ID NO: 42 - RNA, 23 nt, synthetic construct
  source 1..23: mol_type = other RNA
acgatacgtaatcgcatgaaaaa

SEQ ID NO: 43 - RNA, 23 nt, synthetic construct
  source 1..23: mol_type = other RNA
atcgcgataaaacactcgtttaa

SEQ ID NO: 44 - RNA, 23 nt, synthetic construct
  source 1..23: mol_type = other RNA
atcgcgaaaatactagacgatca

SEQ ID NO: 45 - RNA, 23 nt, synthetic construct
  source 1..23: mol_type = other RNA
aacgtacgtaacgctatgaaatt

SEQ ID NO: 46 - RNA, 23 nt, synthetic construct
  source 1..23: mol_type = other RNA
acgcgataaaaatatctgggtcg

SEQ ID NO: 47 - RNA, 23 nt, synthetic construct
  source 1..23: mol_type = other RNA
aacgtacgtattcagacgatagc

SEQ ID NO: 48 - RNA, 23 nt, synthetic construct
  source 1..23: mol_type = other RNA
atcgcgaaaaaacggatgtaacg

SEQ ID NO: 49 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyluridine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
atagacgtgattaatcgcgtt

SEQ ID NO: 50 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylguanosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyluridine-3'-phosphate
  modified_base 15: mod_base = OTHER; note = 2'-O-methylcytidine-3'-phosphate
  modified_base 16: mod_base = OTHER; note = 2'-O-methylguanosine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aagatgatccttatcgcgaat

SEQ ID NO: 51 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-O-methylguanosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
ccgcggattatttatcgcgtt

SEQ ID NO: 52 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methylguanosine-3'-phosphate
  modified_base 7: mod_base = OTHER; note = 2'-deoxy-2'-fluorouridine-3'-phosphate
  modified_base 8: mod_base = OTHER; note = 2'-O-methylcytidine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaagtgtcgaattttcgcgat

SEQ ID NO: 53 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
cacctcctaattaatcgcgtt

SEQ ID NO: 54 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaagctaggtttatacgacgt

SEQ ID NO: 55 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluorouridine-3'-phosphate
  modified_base 8: mod_base = OTHER; note = 2'-O-methylcytidine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
gaggactcaatttatcgcgtt

SEQ ID NO: 56 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaagacgtactttaatcgcgt

SEQ ID NO: 57 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyluridine-3'-phosphate
  modified_base 15: mod_base = OTHER; note = 2'-O-methylcytidine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tcagttgaccatatcgtacgt

SEQ ID NO: 58 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methyladenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tctgtagtggatatacgacgt

SEQ ID NO: 59 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tagtacgactatttatcgcgt

SEQ ID NO: 60 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methylguanosine-3'-phosphate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaagtcgtctttacgtatcgt

SEQ ID NO: 61 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methyladenosine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyladenosine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aacgtacacactaatacgcgt

SEQ ID NO: 62 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaccacatccttatacgacgt

SEQ ID NO: 63 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
atgaacgactttttatcgcgt

SEQ ID NO: 64 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methylcytidine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methylguanosine-3'-phosphate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tttcatgcgattacgtatcgt

SEQ ID NO: 65 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-O-methyladenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
aaacgagtgttttatcgcgat

SEQ ID NO: 66 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyluridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorouridine-3'-phosphate
  modified_base 8: mod_base = OTHER; note = 2'-O-methyladenosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
atcgtctagtattttcgcgat

SEQ ID NO: 67 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methylguanosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methylguanosine-3'-phosphate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tttcatagcgttacgtacgtt

SEQ ID NO: 68 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
acccagatatttttatcgcgt

SEQ ID NO: 69 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluorocytidine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methylguanosine-3'-phosphate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
tatcgtctgaatacgtacgtt

SEQ ID NO: 70 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyladenosine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-deoxy-2'-fluorocytidine-3'-phosphate
  modified_base 8: mod_base = OTHER; note = 2'-O-methylcytidine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate ((2S,4R)-1-[29-[[2- (acetylamino)-2-deoxy-beta-D-galactopyranosyl]oxy]-14,14-b is[[3-[[3-[[5-[[2-(acetylamino)-2-deoxy-beta-D-galactopyra nosyl]oxy]-1-oxopentyl]amino]propyl]amino]-3-oxopropoxy]me thyl]-1,12,19,25-tetraoxo-16-oxa-13,20,24-triazanonacos-1- yl]-4-hydroxy-2-pyrrolidinyl)methyl ester
ttacatccgttttttcgcgat

SEQ ID NO: 71 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyluridine
aacgcgattaatcacgtctatct

SEQ ID NO: 72 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluorocytidine-3'-phosphate
  modified_base 7: mod_base = OTHER; note = 2'-O-methylguanosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyladenosine
attcgcgataaggatcatcttta

SEQ ID NO: 73 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methyluridine
aacgcgataaataatccgcggat

SEQ ID NO: 74 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroguanosine-3'-phosphate
  modified_base 15: mod_base = OTHER; note = 2'-O-methyladenosine-3'-phosphate
  modified_base 16: mod_base = OTHER; note = 2'-deoxy-2'-fluorocytidine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
atcgcgaaaattcgacacttttt

SEQ ID NO: 75 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluoroadenosine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methylguanosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
aacgcgattaattaggaggtggt

SEQ ID NO: 76 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-deoxy-2'-fluorocytidine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methylguanosine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyladenosine
acgtcgtataaacctagctttca

SEQ ID NO: 77 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluoroguanosine-3'-phosphate
  modified_base 15: mod_base = OTHER; note = 2'-O-methyladenosine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methylcytidine-3'-phosphorothioate
  modified_base 22: mod_base = OTHER; note = 2'-O-methylcytidine-3'-phosphorothioate
  modified_base 23: mod_base = OTHER; note = 2'-O-methyladenosine
aacgcgataaattgagtcctcca

SEQ ID NO: 78 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyladenosine-3'-phosphorothioate
  modified_base 23: mod_base = OTHER; note = 2'-O-methyluridine
acgcgattaaagtacgtctttat

SEQ ID NO: 79 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluorocytidine-3'-phosphate
  modified_base 7: mod_base = OTHER; note = 2'-O-methylguanosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
acgtacgatatggtcaactgaaa

SEQ ID NO: 80 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylcytidine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methyluridine
acgtcgtatatccactacagatt

SEQ ID NO: 81 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
acgcgataaatagtcgtactagt

SEQ ID NO: 82 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluoroadenosine-3'-phosphate
  modified_base 7: mod_base = OTHER; note = 2'-O-methylcytidine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
acgatacgtaaagacgacttttt

SEQ ID NO: 83 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluorouridine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
acgcgtattagtgtgtacgtttt

SEQ ID NO: 84 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
acgtcgtataaggatgtggttaa

SEQ ID NO: 85 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyluridine-3'-phosphorothioate
  modified_base 22: mod_base = OTHER; note = 2'-O-methylguanosine-3'-phosphorothioate
  modified_base 23: mod_base = OTHER; note = 2'-O-methyladenosine
acgcgataaaaagtcgttcatga

SEQ ID NO: 86 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluoroadenosine-3'-phosphate
  modified_base 7: mod_base = OTHER; note = 2'-O-methylcytidine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroguanosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
acgatacgtaatcgcatgaaaaa

SEQ ID NO: 87 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
atcgcgataaaacactcgtttaa

SEQ ID NO: 88 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-deoxy-2'-fluorouridine-3'-phosphate
  modified_base 15: mod_base = OTHER; note = 2'-O-methyladenosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyladenosine
atcgcgaaaatactagacgatca

SEQ ID NO: 89 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-deoxy-2'-fluoroadenosine-3'-phosphate
  modified_base 7: mod_base = OTHER; note = 2'-O-methylcytidine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylcytidine-3'-phosphate
  modified_base 13: mod_base = OTHER; note = 2'-O-methylguanosine-3'-phosphate
  modified_base 14: mod_base = OTHER; note = 2'-deoxy-2'-fluorocytidine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methyluridine
aacgtacgtaacgctatgaaatt

SEQ ID NO: 90 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methyluridine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methylguanosine-3'-phosphate
  modified_base 20: mod_base = OTHER; note = 2'-O-methylguanosine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
acgcgataaaaatatctgggtcg

SEQ ID NO: 91 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-deoxy-2'-fluoroadenosine-3'-phosphate
  modified_base 7: mod_base = OTHER; note = 2'-O-methylcytidine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorouridine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylguanosine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
aacgtacgtattcagacgatagc

SEQ ID NO: 92 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methylcytidine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-deoxy-2'-fluoroguanosine-3'-phosphate
  modified_base 15: mod_base = OTHER; note = 2'-O-methylguanosine-3'-phosphate
  modified_base 16: mod_base = OTHER; note = 2'-deoxy-2'-fluoroadenosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
atcgcgaaaaaacggatgtaacg

SEQ ID NO: 93 - RNA, 23 nt, synthetic construct
  source 1..23: mol_type = other RNA
agatagacgtgattaatcgcgtt

SEQ ID NO: 94 - RNA, 23 nt, synthetic construct
  source 1..23: mol_type = other RNA
taaagatgatccttatcgcgaat

SEQ ID NO: 95 - RNA, 23 nt, synthetic construct
  source 1..23: mol_type = other RNA
atccgcggattatttatcgcgtt

SEQ ID NO: 96 - RNA, 23 nt, synthetic construct
  source 1..23: mol_type = other RNA
aaaaagtgtcgaattttcgcgat

SEQ ID NO: 97 - RNA, 23 nt, synthetic construct
  source 1..23: mol_type = other RNA
accacctcctaattaatcgcgtt

SEQ ID NO: 98 - RNA, 23 nt, synthetic construct
  source 1..23: mol_type = other RNA
tgaaagctaggtttatacgacgt

SEQ ID NO: 99 - RNA, 23 nt, synthetic construct
  source 1..23: mol_type = other RNA
ataaagacgtactttaatcgcgt

SEQ ID NO: 100 - RNA, 23 nt, synthetic construct
  source 1..23: mol_type = other RNA
ataaagacgtactttaatcgcgt

SEQ ID NO: 101 - RNA, 23 nt, synthetic construct
  source 1..23: mol_type = other RNA
tttcagttgaccatatcgtacgt

SEQ ID NO: 102 - RNA, 23 nt, synthetic construct
  source 1..23: mol_type = other RNA
aatctgtagtggatatacgacgt

SEQ ID NO: 103 - RNA, 23 nt, synthetic construct
  source 1..23: mol_type = other RNA
actagtacgactatttatcgcgt

SEQ ID NO: 104 - RNA, 23 nt, synthetic construct
  source 1..23: mol_type = other RNA
aaaaagtcgtctttacgtatcgt

SEQ ID NO: 105 - RNA, 23 nt, synthetic construct
  source 1..23: mol_type = other RNA
aaaacgtacacactaatacgcgt

SEQ ID NO: 106 - RNA, 23 nt, synthetic construct
  source 1..23: mol_type = other RNA
ttaaccacatccttatacgacgt

SEQ ID NO: 107 - RNA, 23 nt, synthetic construct
  source 1..23: mol_type = other RNA
tcatgaacgactttttatcgcgt

SEQ ID NO: 108 - RNA, 23 nt, synthetic construct
  source 1..23: mol_type = other RNA
tttttcatgcgattacgtatcgt

SEQ ID NO: 109 - RNA, 23 nt, synthetic construct
  source 1..23: mol_type = other RNA
ttaaacgagtgttttatcgcgat

SEQ ID NO: 110 - RNA, 23 nt, synthetic construct
  source 1..23: mol_type = other RNA
tgatcgtctagtattttcgcgat

SEQ ID NO: 111 - RNA, 23 nt, synthetic construct
  source 1..23: mol_type = other RNA
aatttcatagcgttacgtacgtt

SEQ ID NO: 112 - RNA, 23 nt, synthetic construct
  source 1..23: mol_type = other RNA
cgacccagatatttttatcgcgt

SEQ ID NO: 113 - RNA, 23 nt, synthetic construct
  source 1..23: mol_type = other RNA
gctatcgtctgaatacgtacgtt

SEQ ID NO: 114 - RNA, 23 nt, synthetic construct
  source 1..23: mol_type = other RNA
cgttacatccgttttttcgcgat

SEQ ID NO: 115 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methylcytidine-3'-phosphorothioate
  modified_base 5: mod_base = OTHER; note = 2'-O-methyladenosine-3'-phosphorothioate
  modified_base 6: mod_base = OTHER; note = 2'-O-methylguanosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methyluridine-3'-phosphorothioate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O,4'-C-methylene 5-methyluridine-3'-phosphorothioate
  modified_base 15: mod_base = OTHER; note = 2'-O,4'-C-methylene adenosine-3'-phosphorothioate
  modified_base 16: mod_base = OTHER; note = 2'-O,4'-C-methylene 5-methyluridine-3'-phosphorothioate
  modified_base 17: mod_base = OTHER; note = 2'-O,4'-C-methylene 5-methylcytidine-3'-phosphorothioate
  modified_base 18: mod_base = OTHER; note = 2'-O-methylguanosine-3'-phosphorothioate
  modified_base 19: mod_base = OTHER; note = 2'-O,4'-C-methylene 5-methylcytidine-3'-phosphorothioate
  modified_base 20: mod_base = OTHER; note = 2'-O-methylguanosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
acccagatatttttatcgcga

SEQ ID NO: 116 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O,4'-C-methylene 5-methyluridine-3'-phosphate
  modified_base 4: mod_base = OTHER; note = 2'-O-methyladenosine-3'-phosphorothioate
  modified_base 5: mod_base = OTHER; note = 2'-O,4'-C-methylene 5-methyluridine-3'-phosphate
  modified_base 6: mod_base = OTHER; note = 2'-O,4'-C-methylene 5-methylcytidine-3'-phosphate
  modified_base 7: mod_base = OTHER; note = 2'-O-methylguanosine-3'-phosphorothioate
  modified_base 8: mod_base = OTHER; note = 2'-O,4'-C-methylene 5-methylcytidine-3'-phosphorothioate
  modified_base 9: mod_base = OTHER; note = 2'-O,4'-C-methylene guanosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tttatcgcga

SEQ ID NO: 117 - DNA, 21 bp, synthetic construct
  source 1..21: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methyladenosine-3'-phosphorothioate
  modified_base 6: mod_base = OTHER; note = 2'-O-methylcytidine-3'-phosphorothioate
  modified_base 7: mod_base = OTHER; note = 2'-O-methylguanosine-3'-phosphorothioate
  modified_base 8: mod_base = OTHER; note = 2'-O-methyladenosine-3'-phosphorothioate
  modified_base 9: mod_base = OTHER; note = 2'-O-methylcytidine-3'-phosphorothioate
  modified_base 10: mod_base = OTHER; note = 2'-O-methyluridine-3'-phosphorothioate
  modified_base 11: mod_base = OTHER; note = 2'-O-methyladenosine-3'-phosphorothioate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O,4'-C-methylene 5-methyluridine-3'-phosphorothioate
  modified_base 15: mod_base = OTHER; note = 2'-O,4'-C-methylene adenosine-3'-phosphorothioate
  modified_base 16: mod_base = OTHER; note = 2'-O,4'-C-methylene 5-methyluridine-3'-phosphorothioate
  modified_base 17: mod_base = OTHER; note = 2'-O,4'-C-methylene 5-methylcytidine-3'-phosphorothioate
  modified_base 18: mod_base = OTHER; note = 2'-O-methylguanosine-3'-phosphorothioate
  modified_base 19: mod_base = OTHER; note = 2'-O,4'-C-methylene 5-methylcytidine-3'-phosphorothioate
  modified_base 20: mod_base = OTHER; note = 2'-O-methylguanosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
tagtacgactatttatcgcga

SEQ ID NO: 118 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O,4'-C-methylene 5-methyluridine-3'-phosphate
  modified_base 4: mod_base = OTHER; note = 2'-O-methyladenosine-3'-phosphorothioate
  modified_base 5: mod_base = OTHER; note = 2'-O,4'-C-methylene 5-methyluridine-3'-phosphate
  modified_base 6: mod_base = OTHER; note = 2'-O,4'-C-methylene 5-methylcytidine-3'-phosphate
  modified_base 7: mod_base = OTHER; note = 2'-O-methylguanosine-3'-phosphorothioate
  modified_base 8: mod_base = OTHER; note = 2'-O,4'-C-methylene 5-methylcytidine-3'-phosphorothioate
  modified_base 9: mod_base = OTHER; note = 2'-O,4'-C-methylene guanosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tttatcgcga

SEQ ID NO: 119 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylguanosine-3'-phosphorothioate
  modified_base 5: mod_base = OTHER; note = 2'-O-methyluridine-3'-phosphorothioate
  modified_base 6: mod_base = OTHER; note = 2'-O-methylcytidine-3'-phosphorothioate
  modified_base 7: mod_base = OTHER; note = 2'-O-methylguanosine-3'-phosphorothioate
  modified_base 8: mod_base = OTHER; note = 2'-O-methyluridine-3'-phosphorothioate
  modified_base 9: mod_base = OTHER; note = 2'-O-methylcytidine-3'-phosphorothioate
  modified_base 10: mod_base = OTHER; note = 2'-O-methyluridine-3'-phosphorothioate
  modified_base 11: mod_base = OTHER; note = 2'-O-methyluridine-3'-phosphorothioate
  modified_base 12: mod_base = OTHER; note = 2'-O-methyluridine-3'-phosphorothioate
  modified_base 13: mod_base = OTHER; note = 2'-O-methyladenosine-3'-phosphorothioate
  modified_base 14: mod_base = OTHER; note = 2'-O,4'-C-methylene 5-methylcytidine-3'-phosphorothioate
  modified_base 15: mod_base = OTHER; note = 2'-O,4'-C-methylene guanosine-3'-phosphorothioate
  modified_base 16: mod_base = OTHER; note = 2'-O,4'-C-methylene 5-methyluridine-3'-phosphorothioate
  modified_base 17: mod_base = OTHER; note = 2'-O,4'-C-methylene adenosine-3'-phosphorothioate
  modified_base 18: mod_base = OTHER; note = 2'-O-methyluridine-3'-phosphorothioate
  modified_base 19: mod_base = OTHER; note = 2'-O,4'-C-methylene 5-methylcytidine-3'-phosphorothioate
  modified_base 20: mod_base = OTHER; note = 2'-O-methylguanosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
aaagtcgtctttacgtatcga

SEQ ID NO: 120 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O,4'-C-methylene 5-methylcytidine-3'-phosphate
  modified_base 4: mod_base = OTHER; note = 2'-O-methylguanosine-3'-phosphorothioate
  modified_base 5: mod_base = OTHER; note = 2'-O,4'-C-methylene 5-methyluridine-3'-phosphate
  modified_base 6: mod_base = OTHER; note = 2'-O,4'-C-methylene adenosine-3'-phosphate
  modified_base 7: mod_base = OTHER; note = 2'-O-methyluridine-3'-phosphorothioate
  modified_base 8: mod_base = OTHER; note = 2'-O,4'-C-methylene 5-methylcytidine-3'-phosphorothioate
  modified_base 9: mod_base = OTHER; note = 2'-O,4'-C-methylene guanosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tacgtatcga

SEQ ID NO: 121 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylguanosine-3'-phosphorothioate
  modified_base 5: mod_base = OTHER; note = 2'-O-methyluridine-3'-phosphorothioate
  modified_base 6: mod_base = OTHER; note = 2'-O-methylguanosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methyladenosine-3'-phosphorothioate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O,4'-C-methylene 5-methyluridine-3'-phosphorothioate
  modified_base 15: mod_base = OTHER; note = 2'-O,4'-C-methylene 5-methyluridine-3'-phosphorothioate
  modified_base 16: mod_base = OTHER; note = 2'-O,4'-C-methylene 5-methylcytidine-3'-phosphorothioate
  modified_base 17: mod_base = OTHER; note = 2'-O,4'-C-methylene guanosine-3'-phosphorothioate
  modified_base 18: mod_base = OTHER; note = 2'-O-methylcytidine-3'-phosphorothioate
  modified_base 19: mod_base = OTHER; note = 2'-O,4'-C-methylene guanosine-3'-phosphorothioate
  modified_base 20: mod_base = OTHER; note = 2'-O-methyladenosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
aaagtgtcgaattttcgcgaa

SEQ ID NO: 122 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O,4'-C-methylene 5-methyluridine-3'-phosphate
  modified_base 4: mod_base = OTHER; note = 2'-O-methyluridine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O,4'-C-methylene guanosine-3'-phosphate
  modified_base 7: mod_base = OTHER; note = 2'-O-methylcytidine-3'-phosphorothioate
  modified_base 8: mod_base = OTHER; note = 2'-O,4'-C-methylene guanosine-3'-phosphorothioate
  modified_base 9: mod_base = OTHER; note = 2'-O,4'-C-methylene adenosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
ttttcgcgaa

SEQ ID NO: 123 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylcytidine-3'-phosphorothioate
  modified_base 5: mod_base = OTHER; note = 2'-O-methylguanosine-3'-phosphorothioate
  modified_base 6: mod_base = OTHER; note = 2'-O-methyladenosine-3'-phosphorothioate
  modified_base 7: mod_base = OTHER; note = 2'-O-methylguanosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyluridine-3'-phosphorothioate
  modified_base 11: mod_base = OTHER; note = 2'-O-methyluridine-3'-phosphorothioate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O,4'-C-methylene adenosine-3'-phosphorothioate
  modified_base 15: mod_base = OTHER; note = 2'-O,4'-C-methylene 5-methyluridine-3'-phosphorothioate
  modified_base 16: mod_base = OTHER; note = 2'-O,4'-C-methylene 5-methylcytidine-3'-phosphorothioate
  modified_base 17: mod_base = OTHER; note = 2'-O,4'-C-methylene guanosine-3'-phosphorothioate
  modified_base 18: mod_base = OTHER; note = 2'-O-methylcytidine-3'-phosphorothioate
  modified_base 19: mod_base = OTHER; note = 2'-O,4'-C-methylene guanosine-3'-phosphorothioate
  modified_base 20: mod_base = OTHER; note = 2'-O-methyladenosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
aaacgagtgttttatcgcgaa

SEQ ID NO: 124 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O,4'-C-methylene adenosine-3'-phosphate
  modified_base 4: mod_base = OTHER; note = 2'-O-methyluridine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O,4'-C-methylene guanosine-3'-phosphate
  modified_base 7: mod_base = OTHER; note = 2'-O-methylcytidine-3'-phosphorothioate
  modified_base 8: mod_base = OTHER; note = 2'-O,4'-C-methylene guanosine-3'-phosphorothioate
  modified_base 9: mod_base = OTHER; note = 2'-O,4'-C-methylene adenosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
ttatcgcgaa

SEQ ID NO: 125 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphorothioate
  modified_base 4: mod_base = OTHER; note = 2'-O-methyladenosine-3'-phosphorothioate
  modified_base 5: mod_base = OTHER; note = 2'-O-methyluridine-3'-phosphorothioate
  modified_base 6: mod_base = OTHER; note = 2'-O-methylguanosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylcytidine-3'-phosphorothioate
  modified_base 10: mod_base = OTHER; note = 2'-O-methylcytidine-3'-phosphorothioate
  modified_base 11: mod_base = OTHER; note = 2'-O-methyluridine-3'-phosphorothioate
  modified_base 12: mod_base = OTHER; note = 2'-O-methyluridine-3'-phosphorothioate
  modified_base 13: mod_base = OTHER; note = 2'-O-methyladenosine-3'-phosphorothioate
  modified_base 14: mod_base = OTHER; note = 2'-O,4'-C-methylene 5-methyluridine-3'-phosphorothioate
  modified_base 15: mod_base = OTHER; note = 2'-O,4'-C-methylene 5-methylcytidine-3'-phosphorothioate
  modified_base 16: mod_base = OTHER; note = 2'-O,4'-C-methylene guanosine-3'-phosphorothioate
  modified_base 17: mod_base = OTHER; note = 2'-O,4'-C-methylene 5-methylcytidine-3'-phosphorothioate
  modified_base 18: mod_base = OTHER; note = 2'-O-methylguanosine-3'-phosphorothioate
  modified_base 19: mod_base = OTHER; note = 2'-O,4'-C-methylene adenosine-3'-phosphorothioate
  modified_base 20: mod_base = OTHER; note = 2'-O-methyladenosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
aagatgatccttatcgcgaaa

SEQ ID NO: 126 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O,4'-C-methylene 5-methyluridine-3'-phosphate
  modified_base 4: mod_base = OTHER; note = 2'-O-methylcytidine-3'-phosphorothioate
  modified_base 5: mod_base = OTHER; note = 2'-O,4'-C-methylene guanosine-3'-phosphate
  modified_base 6: mod_base = OTHER; note = 2'-O,4'-C-methylene 5-methylcytidine-3'-phosphate
  modified_base 7: mod_base = OTHER; note = 2'-O-methylguanosine-3'-phosphorothioate
  modified_base 8: mod_base = OTHER; note = 2'-O,4'-C-methylene adenosine-3'-phosphorothioate
  modified_base 9: mod_base = OTHER; note = 2'-O,4'-C-methylene adenosine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tatcgcgaaa

SEQ ID NO: 127 - RNA, 21 nt, synthetic construct
  source 1..21: mol_type = other RNA
  modified_base 14: mod_base = OTHER; note = 5'-methyluridine-3'-phosphate
  modified_base 16: mod_base = OTHER; note = 5'-methyluridine-3'-phosphate
acccagatatttttatcgcga

SEQ ID NO: 128 - RNA, 10 nt, synthetic construct
  source 1..10: mol_type = other RNA
  modified_base 3: mod_base = OTHER; note = 5'-methyluridine-3'-phosphate
  modified_base 5: mod_base = OTHER; note = 5'-methyluridine-3'-phosphate
tttatcgcga

SEQ ID NO: 129 - RNA, 21 nt, synthetic construct
  source 1..21: mol_type = other RNA
  modified_base 14: mod_base = OTHER; note = 5'-methyluridine-3'-phosphate
  modified_base 16: mod_base = OTHER; note = 5'-methyluridine-3'-phosphate
tagtacgactatttatcgcga

SEQ ID NO: 130 - RNA, 10 nt, synthetic construct
  source 1..10: mol_type = other RNA
  modified_base 3: mod_base = OTHER; note = 5'-methyluridine-3'-phosphate
  modified_base 5: mod_base = OTHER; note = 5'-methyluridine-3'-phosphate
tttatcgcga

SEQ ID NO: 131 - RNA, 21 nt, synthetic construct
  source 1..21: mol_type = other RNA
  modified_base 16: mod_base = OTHER; note = 5'-methyluridine-3'-phosphate
aaagtcgtctttacgtatcga

SEQ ID NO: 132 - RNA, 10 nt, synthetic construct
  source 1..10: mol_type = other RNA
  modified_base 5: mod_base = OTHER; note = 5'-methyluridine-3'-phosphate
tacgtatcga

SEQ ID NO: 133 - RNA, 21 nt, synthetic construct
  source 1..21: mol_type = other RNA
  modified_base 14: mod_base = OTHER; note = 5'-methyluridine-3'-phosphate
  modified_base 15: mod_base = OTHER; note = 5'-methyluridine-3'-phosphate
aaagtgtcgaattttcgcgaa

SEQ ID NO: 134 - RNA, 10 nt, synthetic construct
  source 1..10: mol_type = other RNA
  modified_base 3: mod_base = OTHER; note = 5'-methyluridine-3'-phosphate
ttttcgcgaa

SEQ ID NO: 135 - RNA, 21 nt, synthetic construct
  source 1..21: mol_type = other RNA
  modified_base 15: mod_base = OTHER; note = 5'-methyluridine-3'-phosphate
aaacgagtgttttatcgcgaa

SEQ ID NO: 136 - RNA, 10 nt, synthetic construct
  source 1..10: mol_type = other RNA
ttatcgcgaa

SEQ ID NO: 137 - RNA, 21 nt, synthetic construct
  source 1..21: mol_type = other RNA
  modified_base 14: mod_base = OTHER; note = 5'-methyluridine-3'-phosphate
aagatgatccttatcgcgaaa

SEQ ID NO: 138 - RNA, 10 nt, synthetic construct
  source 1..10: mol_type = other RNA
  modified_base 3: mod_base = OTHER; note = 5'-methyluridine-3'-phosphate
tatcgcgaaa

(19) United States
(12) Patent Application Publication
(10) Pub. No.: US 2025/0243490 A1
(43) Pub. Date: Jul. 31, 2025

(54) UNIVERSAL NON-TARGETING SIRNA COMPOSITIONS AND METHODS OF USE THEREOF

(71) Applicant: Alnylam Pharmaceuticals, Inc., Cambridge, MA (US)

(72) Inventors: Megha Subramanian, Cambridge, MA (US); Vasant R. Jadhav, Sharon, MA (US); James D. McIninch, Burlington, MA (US)

(21) Appl. No.: 19/053,526

(22) Filed: Feb. 14, 2025

Related U.S. Application Data
(63) Continuation of application No. PCT/US2023/030461, filed on Aug. 17, 2023.
(60) Provisional application No. 63/398,894, filed on Aug. 18, 2022.

(51) Int. Cl. C12N 15/113

(52) U.S. Cl. CPC C12N 15/113; C12N 2310/14; C12N 2310/313; C12N 2310/321; C12N 2310/3341; C12N 2310/335; C12N 2310/336; C12N 2310/351

(57) ABSTRACT

The present invention provides compositions, systems, and methods for regulating protein expression using iRNA compositions which effect the RNA-induced silencing complex (RISC)-mediated cleavage of a universal RNAi target sequence and REVERSIR compounds which abrogate the activity of such iRNA compositions.

### RELATED APPLICATIONS

This application is a 35 § U.S.C. 111(a) continuation application which claims the benefit of priority to International Application No. PCT/US2023/030461, filed on Aug. 17, 2023, which, in turn, claims the benefit of priority to U.S. Provisional Application No. 63/398,894, filed on Aug. 18, 2022. The entire contents of each of the foregoing applications are incorporated herein by reference.

### SEQUENCE LISTING

The instant application contains a Sequence Listing which has been submitted electronically in XML file format via Patent Center and is hereby incorporated by reference in its entirety. Said XML copy, created on Apr. 14, 2025, is named 121301_20602_SeqList.xml and is 811,370 bytes in size.

### BACKGROUND

Adeno-associated virus (AAV) vectors have emerged as the leading platform for most in vivo gene therapy applications with the potential to achieve years, if not life-long disease treatment option following a single dose.¹ However, one key challenge that has become evident from recent clinical trials of systemic AAV-mediated gene therapy is the wide interindividual variability in therapeutic protein expression at the same vector dose, which could lead to phenotoxicity at supraphysiological transgene levels in some cases.^{41,42} This, along with the difficulty in AAV gene therapy modalities to extrapolate the therapeutically efficacious dose range in humans from preclinical data, underscore the need for clinically translatable approaches to modulate transgene expression after AAV administration.^{2-3,43} The ability to refine transgene dosage to within the targeted therapeutic range or suppress expression in case of adverse events represent important features that could maximize the safety and utility of AAV-based therapies.

RNA interference (RNAi) is an evolutionarily conserved mechanism in which endogenous (microRNA) or exogenous (siRNA, shRNA) short non-coding RNAs downregulate gene expression of mRNA transcripts in sequence-dependent manner.⁴ As a native pathway that leverages an efficient cellular catalytic mechanism, RNAi can be used to achieve robust, durable, and specific silencing of gene transcripts of interest. In recent years, several RNAi-based drugs have been successfully validated in the clinical studies, with demonstrated benefit at low doses and dosing, as infrequent as up to 6 months, compared to alternative gene silencing strategies.⁴⁴ Novel delivery solutions along with highly chemically modified siRNAs have improved potency, durability, and safety and, thus, greatly expanded the reach of RNAi therapeutics, culminating in four approved drugs and several others in clinical development.¹¹⁻¹³ In liver, infrequent delivery of metabolically stabilized siRNA conjugated to N-galactosamine (GalNAc) results in potent gene silencing that persists for months in humans with favorable safety and tolerability profiles.^{14,16} Recent work has also broadened the scope for siRNA delivery to extrahepatic tissues, with conjugation of 2′-O-palmityl (C16) demonstrating wide distribution and durable gene silencing across cell types in central nervous system, eye, and lung.¹⁷ All of these advances for therapeutic silencing of endogenous disease-associated genes via RNAi, also hold the potential for on-demand regulation of exogenously-delivered transgenes in a therapeutic setting.

Given the small footprint of RNAi elements, fully or partially complementary binding sites (typically 19-23 nucleotides) for an interfering RNA may be readily incorporated into viral genomes, typically within the 3′ UTR of the vector-encoded transgene. AAV incorporation of binding sites for endogenous microRNAs has been exploited to improve tissue specificity of gene targeting by selectively attenuating expression in undesired cell types.²⁴⁻²⁶ Prior designs for RNAi-based on-switches have leveraged ligand binding to control the processing of engineered interfering RNAs delivered alongside the therapeutic transgene or to modulate accessibility of endogenous microRNAs to their cognate binding sites on virally-delivered mRNAs.⁵⁻¹⁰ However, their applicability has been hampered by limitations imposed by endogenous microRNA expression levels, risks related to off-targeting, and a lack of non-protein ligands or generalizable aptamers.⁵ In contrast, supplying chemically modified siRNAs exogenously overcomes the reliance on endogenous miRNAs and offers precise and flexible control of dosage. As an alternative to siRNAs which require repeated, though infrequent administration, RNAi via shRNAs that can be stably introduced into AAV vectors in a gene therapy setting allow continuous regulation of the expressed transgene in cis as a single treatment.

While exogenous RNAi modalities may enable low basal expression of transgenes, the versatility of these systems would be greatly enhanced by the ability to achieve reversal of transgene silencing as a means to control therapeutic transgene expression in the on-state. A highly potent and generalizable approach for in vivo control of RNAi pharmacology using short, synthetic single-stranded oligonucleotides known as REVERSIRs²¹ has recently been reported. REVERSIRs functionally abrogate RNAi activity by acting as synthetic high-affinity decoys to sequester RNA-induced silencing complexes (RISC) loaded with complementary siRNA antisense (guide) strands in competition with siRNA target mRNAs. REVERSIRs stably bind to the seed region of the antisense strand, thereby preventing RISC-mediated recognition and degradation of target mRNA transcripts and consequently increasing their translation. With a modular design and generalizable template for length and chemical modifications, REVERSIRs have been shown to potently reverse in vivo gene silencing by multiple siRNA sequences across several targets. The development of REVERSIR as an antidote for RNAi activity represents a valuable tool that may be co-opted to regulate on-states of exogenously delivered transcripts by enabling induction of transgene expression from RNAi-regulated AAV vectors.

Accordingly, there is a need in the art for compositions, systems, and methods that combine RNAi-mediated knockdown with REVERSIR-enabled rescue of gene silencing as a molecular rheostat or switch for AAV-delivered transcripts.

### SUMMARY OF THE INVENTION

The present invention provides compositions, systems, and methods for regulating protein expression using iRNA compositions which effect the RNA-induced silencing complex (RISC)-mediated cleavage of a universal RNAi target sequence and REVERSIR compounds which abrogate the activity of such iRNA compositions. The present invention also provides controllable approaches to fine-tune the magnitude and timing of expression of therapeutic transgenes.

The universal iRNAs of the invention were designed to have favorable thermodynamic properties for RISC loading and RNAi functionality, and to have little to no sequence complementarity to any annotated genes in human, cynomolgus monkey, rat, and mouse transcriptomes. Such universal iRNAs have been demonstrated to be potent RNAi triggers with high on-target specificity, and minimal propensity for off-target gene disruption. In addition, and as described herein, these universal dsRNA agents were shown to modulate expression from exogenous vector-delivery systems without causing undesired off-target silencing within the endogenous transcriptome of humans and mammalian preclinical models. Thus, the use of these universal iRNAs, REVERSIR molecules which abrogate the activity of these universal iRNAs, and systems comprising these universal iRNAs and/or REVERSIR molecules permits refinement of transgene dosage and timing of induction from exogenous vector delivery systems, e.g., AAV vector delivery systems, to thereby provide in vivo gene therapy methods which achieve long-term correction of genetic defects across a wide range of target organs following a single administration.

Accordingly, in one aspect, the present invention provides a universal double stranded ribonucleic acid (dsRNA) agent, comprising a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in Table 2.

In another aspect, the present invention provides a universal double stranded ribonucleic acid (dsRNA) agent, comprising a sense strand and an antisense strand forming a double stranded region, wherein the sense strand comprises at least 15, e.g., 15, 16, 17, 18, 19, 20, or 21, contiguous nucleotides differing by no more than 3, e.g., 3, 2, 1, or 0, nucleotides from any one of the sense strand nucleotide sequences in Table 2 and the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in Table 2.

In one aspect, the present invention provides a universal double stranded ribonucleic acid (dsRNA) agent, comprising a sense strand and an antisense strand forming a double stranded region, wherein the antisense strand comprises a region of complementarity to any one of the target nucleotide sequences in Table 2 or 3.

In one embodiment, the dsRNA agent comprises at least one modified nucleotide.

In one embodiment, substantially all of the nucleotides of the sense strand are modified nucleotides; substantially all of the nucleotides of the antisense strand are modified nucleotides; or substantially all of the nucleotides of the sense strand and substantially all of the nucleotides of the antisense strand are modified nucleotides.

In one embodiment, all of the nucleotides of the sense strand are modified nucleotides; all of the nucleotides of the antisense strand are modified nucleotides; or all of the nucleotides of the sense strand and all of the nucleotides of the antisense strand are modified nucleotides.

In one embodiment, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In another embodiment, the modifications on the modified nucleotides are selected from the group consisting of LNA, HNA, CeNA, 2′-methoxyethyl, 2′-O-alkyl, 2′-O-allyl, 2′-C-allyl, 2′-fluoro, 2′-deoxy, 2′-hydroxyl, and glycol; and combinations thereof.

In yet another embodiment, at least one of the modified nucleotides is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), a nucleotide comprising a 2′ phosphate, and, a vinyl-phosphonate nucleotide; and combinations thereof.

In one embodiment, at least one of the modifications on the modified nucleotides is a thermally destabilizing nucleotide modification.

In one embodiment, the thermally destabilizing nucleotide modification is selected from the group consisting of an abasic modification; a mismatch with the opposing nucleotide in the duplex; and destabilizing sugar modification, a 2′-deoxy modification, an acyclic nucleotide, an unlocked nucleic acids (UNA), and a glycerol nucleic acid (GNA).

The double stranded region may be 19-30 nucleotide pairs in length; 19-25 nucleotide pairs in length; 19-23 nucleotide pairs in length; 23-27 nucleotide pairs in length; or 21-23 nucleotide pairs in length.

In one embodiment, each strand is independently no more than 30 nucleotides in length.

In one embodiment, the sense strand is 21 nucleotides in length and the antisense strand is 23 nucleotides in length.

In one embodiment, the region of complementarity is at least 17 nucleotides in length.

In one embodiment, at least one strand comprises a 3′ overhang of at least 1 nucleotide.

In another embodiment, at least one strand comprises a 3′ overhang of at least 2 nucleotides.

In one embodiment, the universal dsRNA agent further comprises a ligand.

In one embodiment, ligand is conjugated to the 3′ end of the sense strand of the dsRNA agent.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

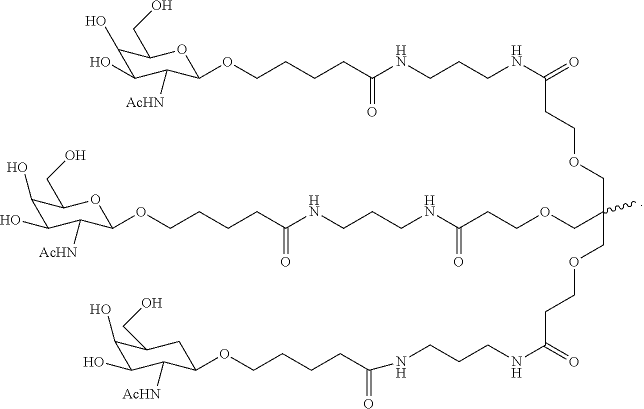 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)CC(CO)C(O)C1O]

In one embodiment, the dsRNA agent is conjugated to the ligand as shown in the following schematic

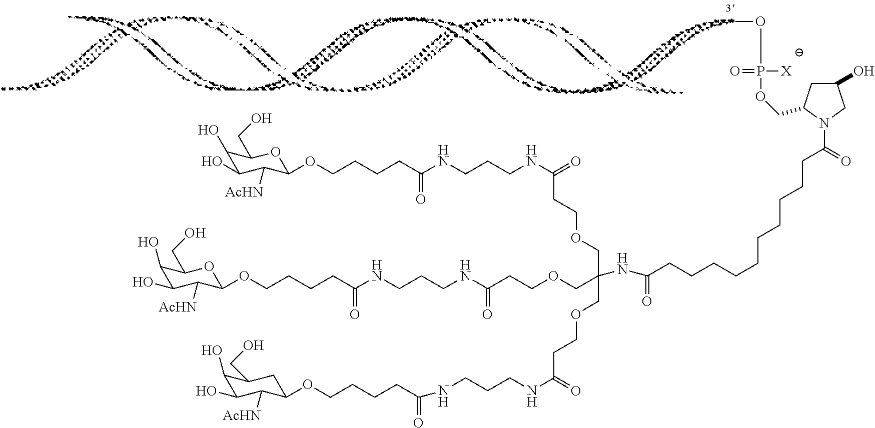

and, wherein X is O or S.

In one embodiment, the X is O.

In one embodiment, wherein the dsRNA agent further comprises at least one phosphorothioate or methylphosphonate internucleotide linkage.

In one embodiment, the phosphorothioate or methylphosphonate internucleotide linkage is at the 3′-terminus of one strand.

In one embodiment, the strand is the antisense strand.

In another embodiment, the strand is the sense strand.

In one embodiment, the phosphorothioate or methylphosphonate internucleotide linkage is at the 5′-terminus of one strand.

In one embodiment, the strand is the antisense strand.

In another embodiment, the strand is the sense strand.

In one embodiment, the phosphorothioate or methylphosphonate internucleotide linkage is at both the 5′- and 3′-terminus of one strand.

In one embodiment, the strand is the antisense strand.

In one embodiment, the base pair at the 1 position of the 5′-end of the antisense strand of the duplex is an AU base pair.

The present invention further provides cells containing the universal dsRNA agents of the invention, as well as vectors comprising the universal dsRNA agents of the invention.

In one embodiment, the vector is an expression vector.

In one embodiment, the vector is a viral vector.

In one embodiment, the viral vector is an adeno-associated (AAV) vector.

In one embodiment, the viral vector is a biscistronic vector.

In one embodiment, the vectors of the invention further comprise a transgene, e.g., a transgene.

Also provided by the present invention are cells containing the vectors of the invention.

The present invention further provides pharmaceutical compositions comprising the universal dsRNA agent of the invention or the vectors of the invention and a pharmaceutically acceptable carrier.

In one embodiment, the dsRNA agent or vector is in an unbuffered solution.

In one embodiment, the unbuffered solution is saline or water.

In another embodiment, the dsRNA agent is in a buffer solution.

In one embodiment, the buffer solution comprises acetate, citrate, prolamine, carbonate, or phosphate or any combination thereof.

In another embodiment, the buffer solution is phosphate buffered saline (PBS).

In one aspect, the present invention provides a REVERSIR compound that abrogates the iRNA activity of the universal dsRNA agent of the invention.

In another aspect, the present invention provides a REVERSIR compound, comprising a single stranded oligonucleotide 6-30 nucleotides in length and comprising a nucleotide sequence which is at least about 90% complementary to any one of the antisense strand nucleotide sequences in Table 2 or Table 3.

In one embodiment, the oligonucleotide is 100% complementary to any one of the antisense strand nucleotide sequences in Table 2 or Table 3.

In one embodiment, the oligonucleotide comprises at least one modified nucleotide.

In one embodiment, substantially all of the nucleotides of the oligonucleotide are modified nucleotides.

In one embodiment, all of the nucleotides of the oligonucleotide are modified nucleotides.

In one embodiment, at least one of the modified nucleotides comprises a modified nucleobase.

In one embodiment, the modified nucleobase is a 5′-methylcytosine.

In one embodiment, at least one of the modified nucleotide comprises a modified sugar.

In one embodiment, the modified sugar is selected from the group consisting of a 2′-O-methoxyethyl modified sugar, a 2′-methoxy modified sugar, a 2′-O-alkyl modified sugar, and a bicyclic sugar.

In one embodiment, the oligonucleotide further comprises a ligand.

In one embodiment, the ligand is conjugated to the 3′ end of the oligonucleotide.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

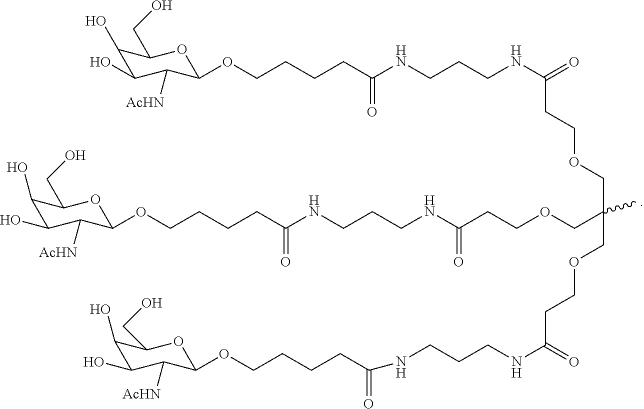 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the oligonucleotide is conjugated to the ligand as shown in the following schematic

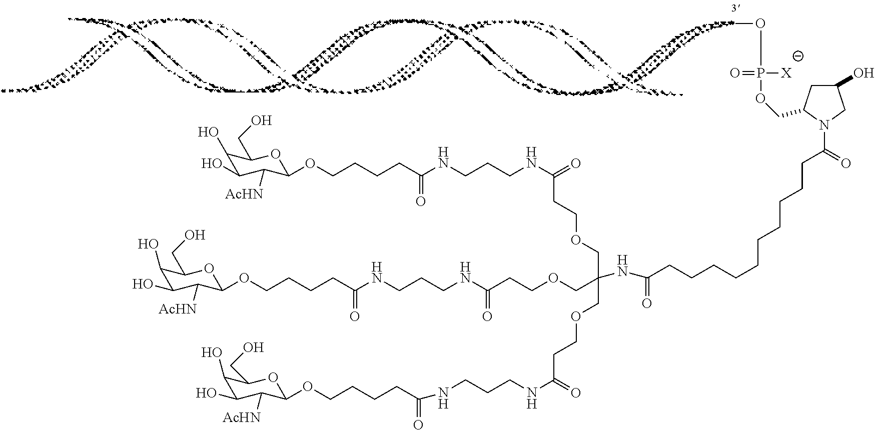

and, wherein X is O or S.

In one embodiment, the X is O.

In one embodiment, the oligonucleotide further comprises at least one phosphorothioate or methylphosphonate internucleotide linkage.

In one embodiment, the oligonucleotide 6-15, 7-11, or 8-10 nucleotides in length.

In another embodiment, the oligonucleotide is 15-25, 17-25, 19-25, or 21-25 nucleotides in length.

The present invention further provides cells comprising a REVERSIR compound of the invention.

In one aspect, the present invention provides a system for on-demand expression of a transgene. Th system includes an expression vector encoding a transgene and comprising a universal iRNA target site; a universal double stranded ribonucleic acid (dsRNA) agent, comprising a sense strand and an antisense strand forming a double stranded region, which recognizes and binds to the universal iRNA target site to thereby inhibit the expression of the transgene; and, optionally, a REVERSIR compound that abrogates the iRNA activity of the universal dsRNA agent to thereby allow the expression of the transgene.

The universal iRNA target site may located in the 5′-untranslated region or the 3′-untranslated region (UTR) of the transgene.

In one embodiment, the universal dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in any one of Table 2 or Table 3.

In one embodiment, the universal dsRNA agent comprises a sense strand and an antisense strand forming a double stranded region, wherein the sense strand comprises at least 15, e.g., 15, 16, 17, 18, 19, 20, or 21, contiguous nucleotides differing by no more than 3, e.g., 3, 2, 1, or 0, nucleotides from any one of the sense strand nucleotide sequences in Table 2 and the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in any one of Table 2 or Table 3.

In one embodiment, the universal dsRNA agent comprises a sense strand and an antisense strand forming a double stranded region, wherein the antisense strand comprises a region of complementarity to any one of the target nucleotide sequences in any one of Table 3 or Table 4.

In one embodiment, the universal dsRNA agent comprises at least one modified nucleotide.

In one embodiment, substantially all of the nucleotides of the sense strand are modified nucleotides; substantially all of the nucleotides of the antisense strand comprise are modified nucleotides; or substantially all of the nucleotides of the sense strand and substantially all of the nucleotides of the antisense strand are modified nucleotides.

In one embodiment, all of the nucleotides of the sense strand are modified nucleotides; all of the nucleotides of the antisense strand are modified nucleotides; or all of the nucleotides of the sense strand and all of the nucleotides of the antisense strand are modified nucleotides.

In one embodiment, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In another embodiment, the modifications on the nucleotides are selected from the group consisting of LNA, HNA, CeNA, 2′-methoxyethyl, 2′-O-alkyl, 2′-O-allyl, 2′-C-allyl, 2′-fluoro, 2′-deoxy, 2′-hydroxyl, and glycol; and combinations thereof.

In still another embodiment, at least one of the modified nucleotides is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), a nucleotide comprising a 2′ phosphate, and, a vinyl-phosphonate nucleotide; and combinations thereof.

In another embodiment, at least one of the modifications on the modified nucleotides is a thermally destabilizing nucleotide modification.

In one embodiment, the thermally destabilizing nucleotide modification is selected from the group consisting of an abasic modification; a mismatch with the opposing nucleotide in the duplex; and destabilizing sugar modification, a 2′-deoxy modification, an acyclic nucleotide, an unlocked nucleic acids (UNA), and a glycerol nucleic acid (GNA).

The double stranded region may be 19-30 nucleotide pairs in length; 19-25 nucleotide pairs in length; 19-23 nucleotide pairs in length; 23-27 nucleotide pairs in length; or 21-23 nucleotide pairs in length.

In one embodiment, each strand is independently no more than 30 nucleotides in length.

In one embodiment, the sense strand is 21 nucleotides in length and the antisense strand is 23 nucleotides in length.

In one embodiment, the region of complementarity is at least 17 nucleotides in length.

In one embodiment, at least one strand comprises a 3′ overhang of at least 1 nucleotide.

In another embodiment, at least one strand comprises a 3′ overhang of at least 2 nucleotides.

In one embodiment, the universal dsRNA agent further comprising a ligand.

In one embodiment, the ligand is conjugated to the 3′ end of the sense strand of the universal dsRNA agent.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

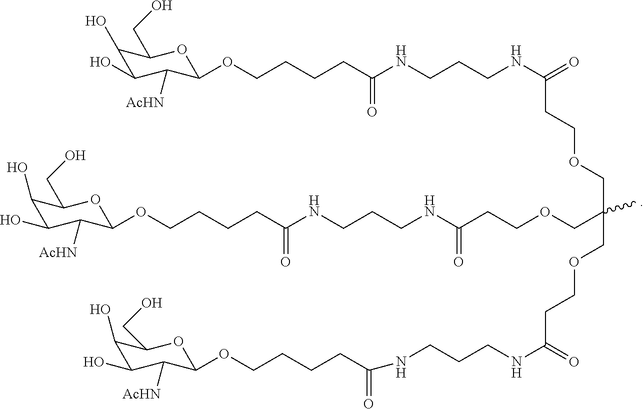 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the universal dsRNA agent is conjugated to the ligand as shown in the following schematic

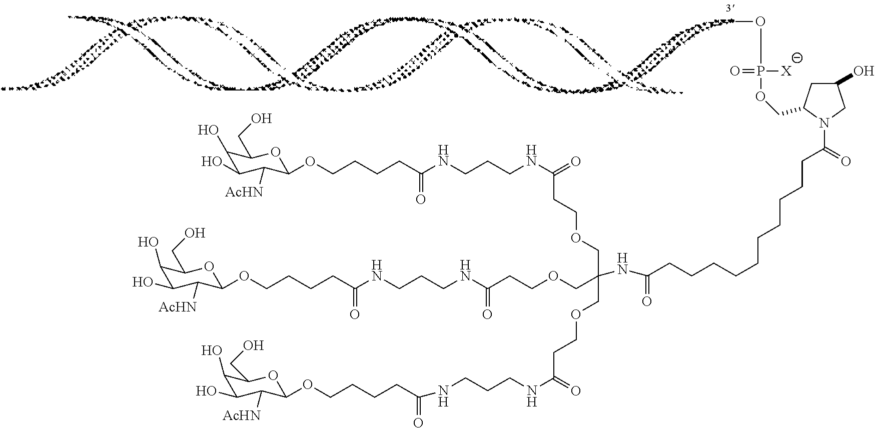

and, wherein X is O or S.

In one embodiment, the X is O.

In one embodiment, the universal dsRNA agent further comprises at least one phosphorothioate or methylphosphonate internucleotide linkage.

In one embodiment, the phosphorothioate or methylphosphonate internucleotide linkage is at the 3′-terminus of one strand.

In one embodiment, the strand is the antisense strand.

In another embodiment, the strand is the sense strand.

In one embodiment, the phosphorothioate or methylphosphonate internucleotide linkage is at the 5′-terminus of one strand.

In one embodiment, the strand is the antisense strand.

In another embodiment, the strand is the sense strand.

In one embodiment, the phosphorothioate or methylphosphonate internucleotide linkage is at both the 5′- and 3′-terminus of one strand.

In one embodiment, the strand is the antisense strand.

In one embodiment, the base pair at the 1 position of the 5′-end of the antisense strand of the duplex is an AU base pair.

In another aspect, the present invention provides a system for on-demand expression of a transgene. The system includes an expression vector encoding a transgene and a double stranded ribonucleic acid (dsRNA) agent targeting the transgene; wherein expression of the transgene is inhibited by the expression of the dsRNA agent targeting the transgene; and, optionally, a REVERSIR compound that abrogates the iRNA activity of the dsRNA agent to thereby allow the expression of the transgene.

In one embodiment, the REVERSIR compound comprises a single stranded oligonucleotide 6-30, e.g., 6-25, 6-20, 8-25, 8-20, 10-25, 10-20, 12-25, 15-25, 17-25, 19-25, 7-23, 19-23, e.g., 6, 7, 8, 9, 10, 11, 12, 13, 14, 15, 16, 17, 18, 19, 20, 21, 22, 23, 24, 25, 26, 27, 28, 29, or 30, nucleotides in length and comprising a nucleotide sequence which is at least about 90% complementary to any one of the antisense strand nucleotide sequences in Table 2 or Table 3.

In one embodiment, the oligonucleotide is 100% complementary to any one of the antisense strand nucleotide sequences in Table 2 or Table 3.

In one embodiment, the oligonucleotide comprises at least one modified nucleotide.

In one embodiment, substantially all of the nucleotides of the oligonucleotide are modified nucleotides.

In one embodiment, all of the nucleotides of the oligonucleotide are modified nucleotides.

In one embodiment, at least one of the modified nucleotides comprises a modified nucleobase.

In one embodiment, the modified nucleobase is a 5′-methylcytosine.

In one embodiment, at least one of the modified nucleotide comprises a modified sugar.

In one embodiment, the modified sugar is selected from the group consisting of a 2′-O-methoxyethyl modified sugar, a 2′-methoxy modified sugar, a 2′-O-alkyl modified sugar, and a bicyclic sugar.

In one embodiment, the REVERSIR compound comprises a ligand.

In one embodiment, the ligand is conjugated to the 3′ end of the oligonucleotide.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

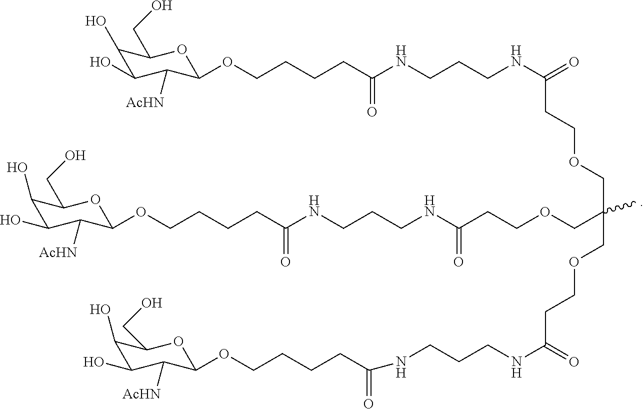 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the oligonucleotide is conjugated to the ligand as shown in the following schematic

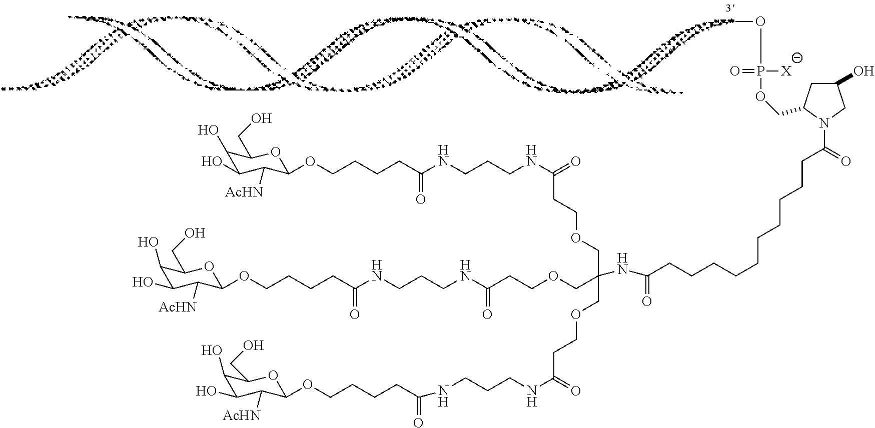

and, wherein X is O or S.

In one embodiment, the X is O.

In one embodiment, the oligonucleotide further comprises at least one phosphorothioate or methylphosphonate internucleotide linkage.

In one embodiment, the oligonucleotide 6-15, 7-11, or 8-10 nucleotides in length.

In one embodiment, the oligonucleotide 15-25, 17-25, 19-25, or 21-25 nucleotides in length.

In one embodiment, the expression vector is a viral vector.

In one embodiment, the viral vector is an adeno-associated (AAV) vector.

In one embodiment, the viral vector is a biscistronic vector.

In one aspect, the present invention provides a method of modulating expression of a transgene in a cell, the method includes contacting the cell with an expression vector encoding a transgene and comprising a universal iRNA target site; contacting the cell with a universal double stranded ribonucleic acid (dsRNA) agent which recognizes and binds to the universal iRNA target site to thereby inhibit expression of the transgene, thereby inhibiting the expression of the transgene; and, optionally, further contacting the cell with a REVERSIR compound that abrogates the iRNA activity of the universal dsRNA agent, thereby allowing expression of the transgene.

In one embodiment, the cell is within a subject.

In another aspect, the present invention provides a method of treating a subject in need thereof. The method includes contacting an expression vector encoding a therapeutic transgene and comprising a universal iRNA target site administered to the subject with a universal double stranded ribonucleic acid (dsRNA) agent which recognizes and binds to the universal iRNA target site to thereby inhibit expression of the transgene, thereby treating the subject.

In one embodiment, the universal dsRNA agent is further contacted with a REVERSIR compound that abrogates the iRNA activity of the universal dsRNA agent to thereby allow the expression of the transgene.

In one aspect, the present invention provides a method of treating a subject in need thereof. The method includes contacting an expression vector encoding a therapeutic transgene and a double stranded ribonucleic acid (dsRNA) agent targeting the transgene administered to the subject with a REVERSIR compound that abrogates the iRNA activity of the dsRNA agent to thereby allow expression of the transgene, thereby treating the subject.

In another aspect, the present invention provides a method of treating a subject in need thereof. The method includes administering to the subject an expression vector encoding a transgene and comprising a universal iRNA target site; allowing expression of the transgene until a desired level of expression has been achieved; and once a desired level of expression of the transgene has been achieved, administering to said subject a universal double stranded ribonucleic acid (dsRNA) agent, comprising a sense strand and an antisense strand forming a double stranded region, which recognizes and binds to the universal iRNA target site to thereby inhibit the expression of the transgene, thereby treating said subject.

In one embodiment, the methods further comprise administering to the subject a REVERSIR compound once the level of the transgene has dropped below a desired level of expression, wherein the REVERSIR compound abrogates the iRNA activity of the universal dsRNA agent to thereby allow the expression of the transgene.

In one embodiment, the universal iRNA target site is located in the 5′-untranslated refion (UTR) or the 3′-untranslated region (UTR) of the transgene.

In one embodiment, the universal dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in any one of Table 2 or Table 3.

In one embodiment, the universal dsRNA agent comprises a sense strand and an antisense strand forming a double stranded region, wherein the sense strand comprises at least 15, e.g., 15, 16, 17, 18, 19, 20, or 21, contiguous nucleotides differing by no more than 3, e.g., 3, 2, 1, or 0, nucleotides from any one of the sense strand nucleotide sequences in Table 2 and the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense strand nucleotide sequences in any one of Table 2 or Table 3.

In one embodiment, the universal dsRNA agent comprises a sense strand and an antisense strand forming a double stranded region, wherein the antisense strand comprises a region of complementarity to any one of the target nucleotide sequences in any one of Table 3 or Table 4.

In one embodiment, the universal dsRNA agent comprises at least one modified nucleotide.

In one embodiment, substantially all of the nucleotides of the sense strand are modified nucleotides; substantially all of the nucleotides of the antisense strand comprise are modified nucleotides; or substantially all of the nucleotides of the sense strand and substantially all of the nucleotides of the antisense strand are modified nucleotides.

In another embodiment, all of the nucleotides of the sense strand are modified nucleotides; all of the nucleotides of the antisense strand are modified nucleotides; or all of the nucleotides of the sense strand and all of the nucleotides of the antisense strand are modified nucleotides.

In one embodiment, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In another embodiment, the modifications on the nucleotides are selected from the group consisting of LNA, HNA, CeNA, 2′-methoxyethyl, 2′-O-alkyl, 2′-O-allyl, 2′-C-allyl, 2′-fluoro, 2′-deoxy, 2′-hydroxyl, and glycol; and combinations thereof.

In one embodiment, at least one of the modified nucleotides is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), a nucleotide comprising a 2′ phosphate, and, a vinyl-phosphonate nucleotide; and combinations thereof.

In another embodiment, at least one of the modifications on the modified nucleotides is a thermally destabilizing nucleotide modification.

In one embodiment, the thermally destabilizing nucleotide modification is selected from the group consisting of an abasic modification; a mismatch with the opposing nucleotide in the duplex; and destabilizing sugar modification, a 2′-deoxy modification, an acyclic nucleotide, an unlocked nucleic acids (UNA), and a glycerol nucleic acid (GNA).

The double stranded region may be 19-30 nucleotide pairs in length; 19-25 nucleotide pairs in length; 19-23 nucleotide pairs in length; 23-27 nucleotide pairs in length; or 21-23 nucleotide pairs in length.

In one embodiment, each strand is independently no more than 30 nucleotides in length.

In one embodiment, the sense strand is 21 nucleotides in length and the antisense strand is 23 nucleotides in length.

In one embodiment, the region of complementarity is at least 17 nucleotides in length.

In one embodiment, at least one strand comprises a 3′ overhang of at least 1 nucleotide.

In another embodiment, at least one strand comprises a 3′ overhang of at least 2 nucleotides.

In one embodiment, the universal dsRNA agent further comprising a ligand.

In one embodiment, the ligand is conjugated to the 3′ end of the sense strand of the universal dsRNA agent.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

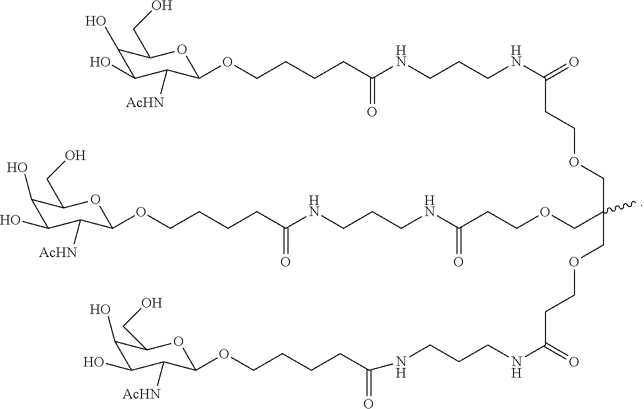 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the universal dsRNA agent is conjugated to the ligand as shown in the following schematic

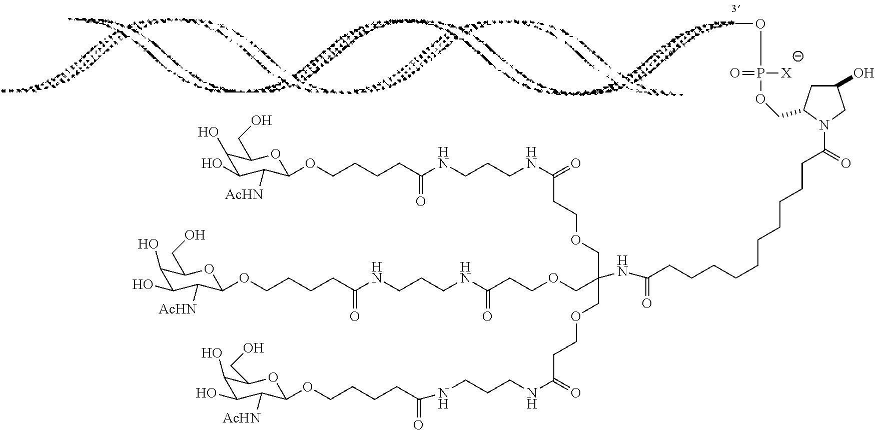

and, wherein X is O or S.

In one embodiment, the X is O.

In one embodiment, the universal dsRNA agent further comprises at least one phosphorothioate or methylphosphonate internucleotide linkage.

In one embodiment, the phosphorothioate or methylphosphonate internucleotide linkage is at the 3′-terminus of one strand.

In one embodiment, the strand is the antisense strand.

In another embodiment, the strand is the sense strand.

In one embodiment, the phosphorothioate or methylphosphonate internucleotide linkage is at the 5′-terminus of one strand.

In one embodiment, the strand is the antisense strand.

In one embodiment, the strand is the sense strand.

In one embodiment, the phosphorothioate or methylphosphonate internucleotide linkage is at both the 5′- and 3′-terminus of one strand.

In one embodiment, the strand is the antisense strand.

In one embodiment, the base pair at the 1 position of the 5′-end of the antisense strand of the duplex is an AU base pair.

In one embodiment, the REVERSIR compound comprises a single stranded oligonucleotide 6-30, e.g., 6-25, 6-20, 8-25, 8-20, 10-25, 10-20, 12-25, 15-25, 17-25, 19-25, 7-23, 19-23, e.g., 6, 7, 8, 9, 10, 11, 12, 13, 14, 15, 16, 17, 18, 19, 20, 21, 22, 23, 24, 25, 26, 27, 28, 29, or 30, nucleotides in length and comprising a nucleotide sequence which is at least about 90% complementary to any one of the antisense strand nucleotide sequences in any one of Table 2 or Table 3.

In one embodiment, the oligonucleotide is 100% complementary to any one of the antisense strand nucleotide sequences in any one of Table 2 or Table 3.

In one embodiment, the oligonucleotide comprises at least one modified nucleotide.

In one embodiment, substantially all of the nucleotides of the oligonucleotide are modified nucleotides.

In another embodiment, all of the nucleotides of the oligonucleotide are modified nucleotides.

In one embodiment, at least one of the modified nucleotides comprises a modified nucleobase.

In one embodiment, the modified nucleobase is a 5′-methylcytosine.

In one embodiment, at least one of the modified nucleotide comprises a modified sugar.

In one embodiment, the modified sugar is selected from the group consisting of a 2′-O-methoxyethyl modified sugar, a 2′-methoxy modified sugar, a 2′-O-alkyl modified sugar, and a bicyclic sugar.

In one embodiment, the REVERSIR compound comprises a ligand.

In one embodiment, the ligand is conjugated to the 3′ end of the oligonucleotide.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

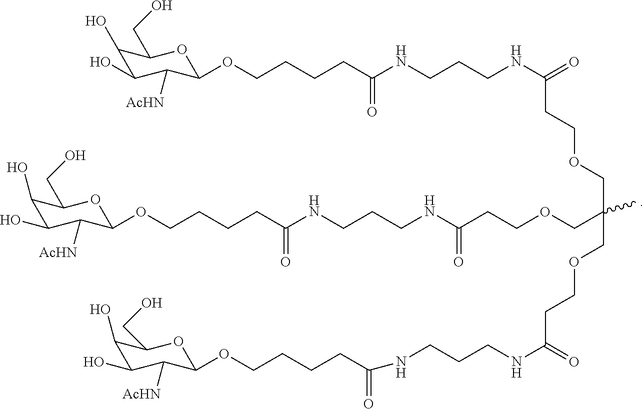 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the oligonucleotide is conjugated to the ligand as shown in the following schematic

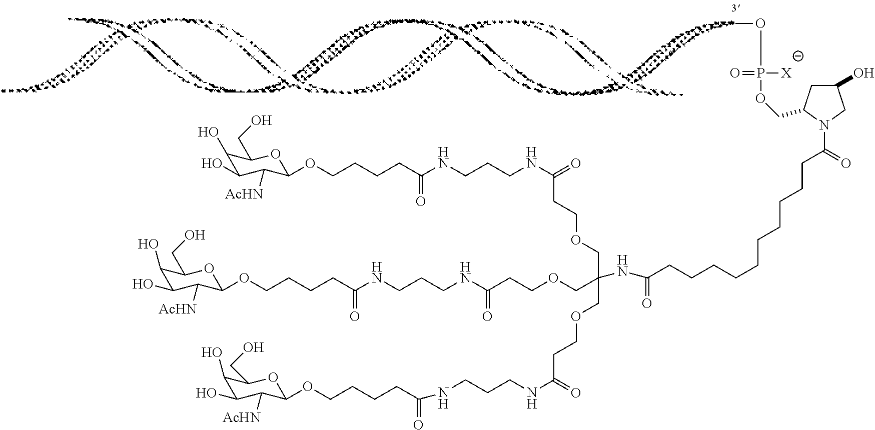

and, wherein X is O or S.

In one embodiment, the X is O.

In one embodiment, the oligonucleotide further comprises at least one phosphorothioate or methylphosphonate internucleotide linkage.

In one embodiment, the oligonucleotide 6-15, 7-11, or 8-10 nucleotides in length.

In one embodiment, the oligonucleotide 15-25, 17-25, 19-25, or 21-25 nucleotides in length.

In one embodiment, the expression vector is a viral vector.

In one embodiment, the viral vector is an adeno-associated (AAV) vector.

In one embodiment, the viral vector is a biscistronic vector.

### BRIEF DESCRIPTION OF THE DRAWINGS

FIGS. 1A-1I depict the recovery of transgene expression from shRNA-regulated self-silencing AAV vectors using REVERSIR.

FIG. 1A is a schematic illustrating an AAV switch using intronically-encoded shRNA for transgene silencing and REVERSIR for transgene induction. shRNA is expressed from a chimeric intron preceding the viral transgene cassette. Following intracellular processing of shRNA, RISC loaded antisense binds to complementary target sites within the rAAV 3′UTR, leading to constitutive cleavage and degradation of AAV-delivered transgene mRNAs. Exogenous delivery of REVERSIR blocks RISC activity, relieving repression of transgene mRNA and inducing protein expression.

FIG. 1B is a viral genome schematic of ssAAV8 self-silencing GLuc reporter vector. Vector expresses intronically-encoded miR-33-embedded TTR shRNA and harbors a cognate (TTR-ts) or scrambled (NT-ts) target site in the 3′ UTR of the GLuc transgene.

FIG. 1C is a graph depicting the timecourse of shRNA-mediated transgene silencing in vitro. HepG2 cells were depicting with the indicated AAV plasmids and cell culture media was collected at each timepoint for quantification of secreted GLuc levels. Media was fully exchanged at every collection, with each line corresponding to accumulated GLuc from a single well since the prior timepoint.

FIG. 1D is a graph depicting the validation of transgene self-suppression with intronic miR-33-containing AAV constructs and induction with REVERSIR in HepG2 cells. Twenty ng of total DNA consisting of a 5:1 ratio of GLuc AAV constructs described in (1B) and an FLuc internal control plasmid were co-transfected with the indicated concentrations of full-length TTR-REVERSIR or chemistry-matched non-targeting (NT) control. Forty-eight hours post-transfection, GLuc and FLuc intensities were assayed in cell culture supernatant and lysate, respectively, and GLuc/FLuc ratios computed to normalize for transfection efficiency.

FIG. 1E is a graph depicting secreted GLuc levels measured in serum collected prior to and at the indicated days following treatment with molar equivalent doses of 9-mer (0.1 mg/kg) or 22-mer (0.2 mg/kg) TTR or NT REVERSIR on D0. Mice were injected with 2×10¹¹ genome copies (GC) of shTTR^{miR-33}/TTR-ts or control shTTR^{miR-33}/NT-ts AAV8 vectors encoding GLuc reporter 2 weeks prior to dosing of REVERSIR compounds.

FIG. 1F is a graph depicting qRT-PCR analysis of GLuc transcript levels in liver tissue at terminal day 47 timepoint, compared to endogenous Gapdh control and plotted relative to shTTR/NT-ts condition set to 100%.

FIG. 1G is a graph depicting longitudinal quantification of serum GLuc levels in mice administered with 0.1 mg/kg or 0.3 mg/kg of a 9-mer tunable TTR REVERSIR (TTR REVERSIR 2) or NT REVERSIR on D0, followed by a second dose on D47.

FIG. 1H is a graph depicting serum EPO concentrations measured at the indicated timepoints by ELISA in mice treated with 0.1 mg/kg 9-mer TTR or NT REVERSIR on D0. Mice were injected with 2×10¹¹ GC of AAV8 virus encoding mouse EPO transgene under control of TTR shRNA with intact (TTR-ts) or non-targeted (NT-ts) binding site in 3′ UTR.

FIG. 1I is a graph depicting serum EPO concentrations at indicated timepoints in mice treated with increasing doses of tunable TTR REVERSIR (TTR REVERSIR 2 at 0.01, 0.03, 0.1, or 0.3 mg/kg) compared to 0.1 mg/kg of NT REVERSIR on D0.

FIGS. 2A-G depict the in vivo regulation of an AAV-delivered reporter transgene by exogenous delivery of siRNA and cognate REVERSIR.

FIG. 2A is a schematic depicting exogenous siRNA approach for AAV transgene modulation. siRNA administration facilitates inactivation or dampening of AAV gene expression through RNAi-mediated degradation of viral transcripts harboring target sites within 3′ UTR. Sequence-specific abrogation of siRNA activity with REVERSIR results in de-repression of viral mRNA transcripts and consequent increases in therapeutic protein expression.

FIG. 2B is a schematic of ssAAV serotype 8 vector carrying a bicistronic expression cassette encoding PMP-22 and GLuc reporter genes. A fully complementary binding site for a TTR siRNA was inserted directly adjacent to the stop codon within the 3′ UTR (left). Six-week-old female C57BL/6 mice were intravenously injected with 2×10¹⁰ genome copies (GC) of AAV. Two weeks after AAV administration, mice were injected subcutaneously (SC) with either vehicle or TTR siRNA at 9 mg/kg (D0). This was followed two weeks later with a single molar equivalent dose of full-length 22-mer (3 mg/kg) or 9-mer TTR REVERSIR (1.6 mg/kg) and compared to vehicle or length-matched NT REVERSIR (D14) as controls. Blood was collected as indicated and terminal liver tissue harvested at D42 (right).

FIG. 2C is a graph depicting quantification of serum GLuc levels at indicated timepoints normalized to pre-dose for each animal.

FIG. 2D is a graph depicting qRT-PCR analyses of GLuc transcript levels in terminal liver tissue at D42 normalized to Gapdh control and plotted relative to PBS condition set to 100%.

FIG. 2E is a graph depicting serum GLuc levels at D21 in mice transduced with 2×10¹¹ GC of AAV shown in (2B) and treated with TTR siRNA (9 mg/kg; D0), followed by varying doses of 9-mer TTR REVERSIR or NT REVERSIR (D14) at high dose alone as control.

FIG. 2F depict an AAV vector schematic for assessment of shRNA-based modulation of AAV-hANGPTL3 transgene (left) and a graph of plasma hANGPTL3 protein concentrations assessed over the indicated timecourse by ELISA (right). C57BL/6 mice had received intravenous administration of 1.5×10¹¹ GCs of AAV8 vectors carrying the human ANGPTL3 coding region with a target site for a GLuc siRNA within the 3′ UTR. Two weeks later, mice were treated with 9 mg/kg GLuc siRNA for 14 days, after which 1.5 mg/kg 9-mer GLuc REVERSIR or NT REVERSIR was administered. Bleeds were performed at the timepoints shown.

FIG. 2G depict an AAV vector schematic for siRNA-mediated regulation of human Factor XII (hF12)-GLuc transgene (left) and a graph of serum GLuc intensity measured at the indicated timepoints and plotted relative to pre-treatment with siRNA (right). Mice were injected with 2×10¹¹ GCs of an IRES-containing bicistronic vector encoding hF12 and GLuc, with a TTR siRNA binding site in the 3′ UTR. Mice were administered with 9 mg/kg TTR siRNA, then after 2 weeks given 156 mg/kg TTR REVERSIR or NT REVERSIR, with blood draws performed as indicated.

FIGS. 3A-3D depict the in vitro characterization of on- and off-target activity of transgene regulator siRNA sequences.

FIG. 3A are graphs depicting the on-target silencing efficacy (solid black line) of three lead transgene regulator siRNA sequences as assayed by co-transfection of serially titrated doses of siRNA with dual luciferase sensors containing a perfectly matched binding site. Seed-mediated off-target repression was similarly assessed by dose-response activity of siRNA in the presence of luciferase reporters bearing either 1 (medium gray dashed line) or 4 tandem (light grey dashed line) seed-matched target sites. RLuc/FLuc ratios were normalized to the mock-transfected control (no siRNA) condition set at 100%, and plotted as mean of 3-6 replicates±SEM.

FIG. 3B are Bland-Altman plots (MA plots) depicting differential gene expression analysis of RNA-seq data obtained from transfection of transgene regulator siRNAs in Hep3B cells (top; 10 nM dose harvested at 24 h) and primary mouse hepatocytes (bottom; 50 nM dose harvested at 48 h) Dots represent individual transcripts, average normalized read counts across replicates, and log₂ fold change relative to mock transfected controls. ‘Black’-colored dots denote genes with significant differential expression (FDR<0.05) but no canonical seed-matched sites within their 3′ UTRs (8-mer, 7mer-m8, and 7mer-A1). ‘Dark gray’ dots represent genes that contain canonical 3′ UTR seed-matched binding sites but are not significantly differentially expressed. (N=4 technical replicates).

FIG. 3C are tables showing differential gene expression analyses of in vitro RNAseq data from transfection of transgene regulator siRNAs in mouse (primary mouse hepatocytes; top) and human (Hep3B; bottom) hepatic cells.

FIG. 3D are graphs depicting serum alanine aminotransferase (ALT) and glutamate dehydrogenase (GLDH) at necropsy (D16) in rats that received 3 once weekly injections (qw×3) of indicated transgene regulator siRNAs (TR-siRNA) at 30 or 100 mg/kg dose. N=4 males (6-8 weeks old) per group; qw weekly dosing.

FIGS. 4A-4E depict additional in vitro and in vivo analyses supporting AAV regulatory switch leveraging intronically-expressed shRNA and REVERSIR.

FIG. 4A is a schematic of a marker construct and a graph depicting in vitro assessment of REVERSIR-mediated reversal of target silencing by miRNA-mediated shRNA in the dual luciferase reporter assay. Cos7 cells were co-transfected for 48 hours with luciferase reporter plasmid and GFP marker constructs expressing miR-30E-embedded TTR or NT shRNAs, along with increasing concentrations of 22-mer TTR or matched NT REVERSIR.

FIG. 4B is a schematic of a marker construct and a graph depicting in vitro assessment of REVERSIR-mediated reversal of target silencing by miRNA-mediated shRNA in the dual luciferase reporter assay. Cos7 cells were co-transfected for 48 hours with luciferase reporter plasmid and GFP marker constructs expressing miR-33-embedded TTR or NT shRNAs, along with increasing concentrations of 22-mer TTR or matched NT REVERSIR.

FIG. 4C is a graph showing validation of GLuc transgene suppression with intronic miR-30E-shRNA-containing self-silencing AAV constructs and subsequent induction with increasing doses of REVERSIR in HepG2 cells. AAV constructs were co-transfected with FLuc control plasmids for normalization at 5:1 molar ratio. GLuc and FLuc intensities were assayed in cell culture supernatant and lysate, respectively, and GLuc/FLuc ratios expressed as % relative to shNT-expressing AAV plasmid.

FIG. 4D is a graph depicting quantification of GLuc mRNA levels by qRT-PCR in HepG2 cells 48 h following transfection with miR-33-containing self-silencing AAV plasmids and REVERSIR. GLuc transcript levels were normalized to Fluc mRNA as internal control.

FIG. 4E is a graph showing successful in vivo knockdown of endogenous TTR protein levels by intronic expression of shTTR^{miR-33} and subsequent recovery back to baseline with exogenous administration of TTR REVERSIR but not NT REVERSIR.

FIGS. 5A-5F depict additional in vitro and in vivo analyses supporting AAV regulatory switch leveraging exogenous siRNA and REVERSIR.

FIGS. 5A-5C are graphs depicting data from individual animals or additional groups tested as part of the study shown in FIG. 2B-D. AAV injections, timing of test article dosing, and blood collections were conducted as described in FIG. 2B. PBS and 9 mg/kg siRNA conditions are identical to those shown in main figure.

FIGS. 5A-5F are graphs depicting data from individual animals or additional groups tested as part of the study shown in FIG. 2B-D. AAV injections, timing of test article dosing, and blood collections were conducted as described in FIG. 2B. PBS and 9 mg/kg siRNA conditions are identical to those shown in main figure.

FIG. 5A is a graph depicting sustained dose-dependent knockdown of serum GLuc levels in AAV-injected mice treated with 1, 3, and 9 mg/kg TTR siRNA as compared to PBS control.

FIG. 5B are graphs depicting longitudinal measurement of serum GLuc levels in AAV-injected mice dosed with 3 mg/kg TTR siRNA and subsequently with vehicle or 1 mg/kg dose of the specified REVERSIR (left). Averaged data in FIG. 2C presented as spaghetti graph plotting serum GLuc changes over time relative to pre-dose for each individual animal treated with 9 mg/kg TTR siRNA followed by 3 mg/kg of the specified REVERSIR molecules (right).

FIG. 5C is a graph depicting a positive control demonstrating expected silencing of endogenous TTR mRNA with 9 mg/kg TTR siRNA and complete reversal of knockdown with 3 mg/kg 22-mer and 9-mer TTR REVERSIR but not corresponding NT REVERSIR.

FIG. 5D are graphs depicting on-target silencing activity of GLuc siRNA in dual luciferase reporter system (left). Normalized luciferase activity 48 h following co-transfection of Cos7 cells with 10 nM GLuc siRNA and increasing doses of 22-mer or 9-mer GLuc REVERSIR (right).

FIG. 5E is a spaghetti plot showing responses of individual animals that were averaged by condition to generate the graph shown in FIG. 2F. The graph on the right shows hANGPTL3 concentration over time in one animal that was identified as a significant outlier by Grubb's test due to low level of AAV transduction and, thus, omitted from main figure.

FIG. 5F is a spaghetti plot showing responses of individual animals that were averaged by condition to generate the graph shown in FIG. 2G.

FIGS. 6A-6B depict the lack of seed-mediated off-target effects from transgene regulator siRNAs

FIG. 6A depicts a cumulative distribution function (CDF) plot showing transcriptional changes after transfection of transgene regulator siRNAs in Hep3B at 10 nM for 24 h. Each line represents the cumulative distribution of expression change among target genes with the specified seed matches (8mer, 7mer-m8, and 7mer-A1) to the siRNA antisense strand (top) or sense strand (bottom) within the 3′UTR, as compared to genes bearing no such canonical seed match sites (background). The black line represents background genes lacking the specified seed matches while the varied gray lines represent genes with at least one seed match, split by the strength of the binding site (dark gray=8mer, medium gray=7mer-m8, light gray=m7mer-A1). Delta values reflect the magnitude of CDF shifts relative to background. N=4 technical replicates.

FIG. 6B depicts a cumulative distribution function (CDF) plot showing transcriptional changes after transfection of transgene regulator siRNAs in primary mouse hepatocytes at 50 nM for 48 h. Each line represents the cumulative distribution of expression change among target genes with the specified seed matches (8mer, 7mer-m8, and 7mer-A1) to the siRNA antisense strand (top) or sense strand (bottom) within the 3′UTR, as compared to genes bearing no such canonical seed match sites (background). The black line represents background genes lacking the specified seed matches while the varied gray lines represent genes with at least one seed match, split by the strength of the binding site (dark gray=8mer, medium gray=7mer-m8, light gray=m7mer-A1). Delta values reflect the magnitude of CDF shifts relative to background. N=4 technical replicates

FIG. 7 are graphs depicting the lack of liver function test (LFT) elevations in rat toxicity studies of transgene regulator siRNAs. In particular, tge graphs depict serum levels of aspartate aminotransferase (AST), albumin (ALB), alkaline phosphatase (ALP), and total protein (TP) at necropsy (D16) in rats that received 3 once weekly injections (qw×3) of indicated transgene regulator siRNAs (TR-siRNA) at 30 or 100 mg/kg dose. N=4 males (6-8 weeks old) per group; qw weekly dosing.

### DETAILED DESCRIPTION OF THE INVENTION

The present invention provides compositions, systems, and methods for regulating protein expression using iRNA compositions which effect the RNA-induced silencing complex (RISC)-mediated cleavage of a universal RNAi target sequence mRNA and REVERSIR compounds which abrogate the activity of such iRNA compositions.

The universal iRNAs of the invention were designed to have favorable thermodynamic properties for RISC loading and RNAi functionality, and to have little to no sequence complementarity to any annotated genes in human, cynomolgus monkey, rat, and mouse transcriptomes. Such universal iRNAs have been demonstrated to be potent RNAi triggers with high on-target specificity, and minimal propensity for off-target gene disruption. In addition, and as described herein, these universal dsRNA agent were shown to modulate expression from exogenous vector-delivery systems without causing undesired off-target silencing within the endogenous transcriptome of humans and mammalian preclinical models. Thus, the use of these universal iRNAs, REVERSIR molecules which abrogate the activity of these universal iRNAs, and systems comprising these universal iRNAs and/or REVERSIR molecules permits refinement of transgene dosage and timing of induction from exogenous vector delivery systems, e.g., AAV vector delivery systems, to thereby provide in vivo gene therapy methods which achieve long-term correction of genetic defects across a wide range of target organs following a single administration.

The iRNAs of the invention include an RNA strand (the antisense strand) having a region which is up to about 30 nucleotides or less in length, e.g., 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of a universal target.

In certain embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of a universal target. In some embodiments, such iRNA agents having longer length antisense strands may, for example, include a second RNA strand (the sense strand) of 20-60 nucleotides in length wherein the sense and antisense strands form a duplex of 18-30 contiguous nucleotides.

The following detailed description discloses how to make and use compositions containing iRNAs to inhibit the expression of a universal target sequence, REVERSIR compounds which abrogate the activity of such iRNAs, as well as systems, uses, and methods for treating subjects in need thereof.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

As used herein, “target sequence” refers to a contiguous portion of the nucleotide sequence of an mRNA molecule formed during the transcription of a universal target sequence, including mRNA that is a product of RNA processing of a primary transcription product. In one embodiment, the target portion of the sequence will be at least long enough to serve as a substrate for iRNA-directed cleavage at or near that portion of the nucleotide sequence of an mRNA molecule formed during the transcription of a universal target sequence.

A “universal target sequence” is a nucleotide sequence which has favorable thermodynamic properties for RISC loading and RNAi functionality, and little to no sequence complementarity to any annotated genes in human, cynomolgus monkey, rat, and mouse transcriptomes. Such universal iRNAs have been demonstrated to be potent RNAi triggers with high on-target specificity, and minimal propensity for off-target gene disruption.

The nucleotide sequences of exemplary universal target sequences are provided in Tables 3 and 4 below.

The target sequence may be about 19-36 nucleotides in length. For example, the target sequence can be about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a universal target mRNA sequence, e.g., in a cell, e.g., a cell within a subject, such as a mammalian subject.

In one embodiment, an RNAi agent of the invention includes a single stranded RNA that interacts with a target RNA sequence, e.g., a universal target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target sequence, i.e., a universal target sequence. Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a universal target mRNA sequence. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this specification, an “iRNA” may include ribonucleotides with chemical modifications; an iRNA may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “iRNA” or “RNAi agent” for the purposes of this specification and claims.

In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 19 to 36 base pairs in length, e.g., about 19-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, 3, 4, 5, 6, 7, 8, 9, 10, 20, 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, an iRNA agent of the invention is a dsRNA, each strand of which comprises 19-23 nucleotides, that interacts with a target RNA sequence, e.g., a universal target mRNA sequence, to direct cleavage of the target RNA.

In some embodiments, an iRNA of the invention is a dsRNA of 24-30 nucleotides that interacts with a target RNA sequence, e.g., a universal target mRNA sequence, to direct the cleavage of the target RNA.

As used herein, the term “nucleotide overhang” refers to at least one unpaired nucleotide that protrudes from the duplex structure of a double stranded iRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotides, e.g., a 1, 2, 3, 4, 5, 6, 7, 8, 9, or 10 nucleotide, overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′ end of the antisense strand of the duplex. In certain embodiments, an extended overhang is present on the 5′ end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an iRNA, e.g., a dsRNA, which includes a region that is substantially complementary to a target sequence, e.g., a universal target mRNA.

As used herein, the term “region of complementarity” refers to the region on the antisense strand that is substantially complementary to a sequence, for example a target sequence, e.g., a universal target nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the iRNA. In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a universal target sequence, generally does not contain any mismatch within the central 13 nucleotides. The methods described herein or methods known in the art can be used to determine whether an RNAi agent containing a mismatch to a target sequence is effective in inhibiting the expression of a universal target mRNA sequence. Consideration of the efficacy of RNAi agents with mismatches in inhibiting expression of a universal target sequence is important, especially if the particular region of complementarity in a universal target sequence is known to have polymorphic sequence variation within the population.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucletoides or polynucleotides, such as the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a universal target sequence). For example, a polynucleotide is complementary to at least a part of a universal target mRNA sequence if the sequence is substantially complementary to a non-interrupted portion of an mRNA encoding a universal target sequence.

Accordingly, in some embodiments, the antisense polynucleotides disclosed herein are fully complementary to the target universal sequence. In other embodiments, the antisense polynucleotides disclosed herein are substantially complementary to the target universal sequence and comprise a contiguous nucleotide sequence which is at least 80% complementary over its entire length to the nucleotide sequence of any one of the target nucleotide sequences in any one of Tables 3 and 4, or a fragment of any one of the target nucleotide sequences in any one of Tables 3 and 4, such as about 85%, about 90%, about 91%, about 92%, about 93%, about 94%, about 95%, about 96%, about 97%, about 98%, or about 99% complementary.

In other embodiments, the antisense polynucleotides disclosed herein are substantially complementary to the target universal sequence and comprise a contiguous nucleotide sequence which is at least about 80% complementary over its entire length to any one of the sense strand nucleotide sequences in any one of any one of Tables 2 and 3, or a fragment of any one of the sense strand nucleotide sequences in any one of Tables 2 and 3, such as about 85%, about 90%, about 91%, about 92%, about 93%, about 94%, about 95%, about 96%, about 97%, about 98%, about 99%, or 100% complementary.

In some embodiments, an iRNA of the invention includes a sense strand that is substantially complementary to an antisense polynucleotide which, in turn, is complementary to a target universal sequence, and wherein the sense strand polynucleotide comprises a contiguous nucleotide sequence which is at least about 80% complementary over its entire length to any one of the antisense strand nucleotide sequences in any one of any one of Tables 2 and 3, or a fragment of any one of the antisense strand nucleotide sequences in any one of Tables 2 and 3, such as about 85%, about 90%, about 91%, about 92%, about 93%, about 94%, about 95%, about 96%, about 97%, about 98%, about 99%, or 100% complementary.

In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In certain embodiments of the instant disclosure, inclusion of a deoxy-nucleotide if present within an RNAi agent can be considered to constitute a modified nucleotide.

In an aspect of the invention, an agent for use in the methods and compositions of the invention is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

As used herein, the term “REVERSIR compound” refers to an oligomeric compound that is complementary to and capable of hybridizing with (targeted to) at least one strand of a conjugated or unconjugated universal dsRNA agent.

A “REVERSIR compound” decreases or abrogates the intensity and/or duration of activity of a universal dsRNA agent attributable to hybridization of a REVERSIR compound to one of the strands of the universal dsRNA agent.

The REVERSIR compounds disclosed herein are particularly effective in reducing the activity of siRNAs. For example, the REVERSIR compounds disclosed herein can reduce the activity of an siRNA by at least about 50%, or at least about 60%, or at least about 70%, or at least about 80%, or at least about 90%, or at least about 95%, or at least about 97%, or at least about 99% or up to and including a 100% decrease (i.e., absent level as compared to a reference sample), or any decrease between 50-100% as compared to a reference level. The reference level can be siRNA activity in absence of the REVERSIR compound.

In some embodiments, the REVERSIR compounds describe herein can reduce the activity of a universal dsRNA agent by at least 75%, for example by 80%, 85%, 90%, 95% or more and up to and including complete reduction or inhibition of siRNA activity. By complete reduction of siRNA activity is meant a reduction of the siRNA activity by at least 80% relative to a reference level.

The phrase “contacting a cell with an iRNA,” such as a dsRNA, as used herein, includes contacting a cell by any possible means. Contacting a cell with an iRNA includes contacting a cell in vitro with the iRNA or contacting a cell in vivo with the iRNA. The contacting may be done directly or indirectly. Thus, for example, the iRNA may be put into physical contact with the cell by the individual performing the method, or alternatively, the iRNA may be put into a situation that will permit or cause it to subsequently come into contact with the cell.

Contacting a cell in vitro may be done, for example, by incubating the cell with the iRNA. Contacting a cell in vivo may be done, for example, by injecting the iRNA into or near the tissue where the cell is located, or by injecting the iRNA into another area, e.g., the bloodstream or the subcutaneous space, such that the agent will subsequently reach the tissue where the cell to be contacted is located. For example, the iRNA may contain or be coupled to a ligand, e.g., GalNAc, that directs the iRNA to a site of interest, e.g., the liver. Combinations of in vitro and in vivo methods of contacting are also possible. For example, a cell may also be contacted in vitro with an iRNA and subsequently transplanted into a subject.

In certain embodiments, contacting a cell with an iRNA includes “introducing” or “delivering the iRNA into the cell” by facilitating or effecting uptake or absorption into the cell. Absorption or uptake of an iRNA can occur through unaided diffusion or active cellular processes, or by auxiliary agents or devices. Introducing an iRNA into a cell may be in vitro or in vivo. For example, for in vivo introduction, iRNA can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the universal target sequence, either endogenously or heterologously. In an embodiment, the subject is a human. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result, such as reducing at least one sign or symptom of a disorder in a subject or improvement of at least one sign or symptom of the disease or condition.

“Treatment” can also mean prolonging survival as compared to expected survival in the absence of treatment. The term “lower” refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” also includes a decrease to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in bodyweight between an obese individual and an individual having a weight accepted within the range of normal.

As used herein, “prevention” or “preventing,” when used in reference to a disease, disorder or condition thereof, refers to a reduction in the likelihood that a subject will develop a symptom associated with such a disease, disorder, or condition, e.g., a symptom of the disease. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

“Therapeutically effective amount,” as used herein, is intended to include the amount of an RNAi agent or compound that, when administered to a subject, is sufficient to effect treatment of the disease (e.g., by diminishing, ameliorating, or maintaining the existing disease or one or more symptoms of disease). The “therapeutically effective amount” may vary depending on the agent or compound, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an agent or compound that, when administered to a subject, is sufficient to prevent or ameliorate the disease or one or more symptoms of the disease. Ameliorating the disease includes slowing the course of the disease or reducing the severity of later-developing disease. The “prophylactically effective amount” may vary depending on the agent or compound, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an agent or compound that produces some desired effect at a reasonable benefit/risk ratio applicable to any treatment. The agent or compound employed in the methods of the present invention may be administered in a sufficient amount to produce a reasonable benefit/risk ratio applicable to such treatment.

The phrase “pharmaceutically acceptable” is employed herein to refer to those compounds, materials (including salts), compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

### II. Universal iRNAs of the Invention

The present invention provides iRNAs which inhibit the expression of a universal target sequence. In certain embodiments, the iRNA includes double stranded ribonucleic acid (dsRNA) molecules for inhibiting the expression of a universal target sequence in a cell, such as a cell within a subject, e.g., a mammal, such as a human in need of treatment. The dsRNAi agent includes an antisense strand having a region of complementarity which is complementary to at least a part of an mRNA formed in the expression of a universal target sequence. The region of complementarity is about 19-30 nucleotides in length (e.g., about 30, 29, 28, 27, 26, 25, 24, 23, 22, 21, 20, or 19 nucleotides in length).

Upon contact with a cell expressing the universal target sequence, the iRNA inhibits the expression of the universal target sequence by at least about 50% as assayed by, for example, a PCR or branched DNA (bDNA)-based method, or by a protein-based method, such as by immunofluorescence analysis, using, for example, western blotting or flow cytometric techniques. In certain embodiments, inhibition of expression is determined by the qPCR method provided in the examples herein with the siRNA at, e.g., a 10 nM concentration, in an appropriate organism cell line provided therein. In certain embodiments, inhibition of expression in vivo is determined by knockdown of the human universal target sequence mRNA in a rodent expressing the human mRNA, e.g., a mouse or an AAV-infected mouse expressing the human universal target mRNA, e.g., when administered as single dose, e.g., at 3 mg/kg at the nadir of RNA expression.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a universal target sequence. The target sequence can be derived from the sequence of an mRNA formed during the expression of a universal target sequence. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target expression of a universal target sequence is not generated in the target cell by cleavage of a larger dsRNA.

A dsRNA as described herein can further include one or more single-stranded nucleotide overhangs, e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In an aspect, a dsRNA of the invention includes at least two nucleotide sequences, a sense sequence and an anti-sense sequence. The sense strand is selected from the group of sequences provided in any one of Tables 2-3, and the corresponding antisense strand of the sense strand is selected from the group of sequences of any one of Tables 2-3. In this aspect, one of the two sequences is complementary to the other of the two sequences, with one of the sequences being substantially complementary to a sequence of an mRNA generated in the expression of a universal target sequence. As such, in this aspect, a dsRNA will include two oligonucleotides, where one oligonucleotide is described as the sense strand in any one of Tables 2-3, and the second oligonucleotide is described as the corresponding antisense strand of the sense strand in any one of Tables 2-3.

In certain embodiments, the substantially complementary sequences of the dsRNA are contained on separate oligonucleotides. In other embodiments, the substantially complementary sequences of the dsRNA are contained on a single oligonucleotide.

In one embodiment, the antisense strand comprises at least 15, e.g., 15, 16, 17, 18, 19, or 20, contiguous nucleotides differing by no more than 0, 1, 2, or 3 nucleotides from any one of the antisense strand nucleotide sequences in any one of Tables 2-3.

It will be understood that, although the sequences in, for example, Table 2, are not described as modified or conjugated sequences, the RNA of the iRNA of the invention e.g., a dsRNA of the invention, may comprise any one of the sequences set forth in any one of Tables 2-3 that is un-modified, un-conjugated, or modified or conjugated differently than described therein. In other words, the invention encompasses dsRNAs of Tables 2-3 which are un-modified, un-conjugated, modified, or conjugated, as described herein.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any one of Tables 2-3. dsRNAs described herein can include at least one strand of a length of minimally 21 nucleotides. It can be reasonably expected that shorter duplexes having any one of the sequences in any one of Tables 2-3 minus only a few nucleotides on one or both ends can be similarly effective as compared to the dsRNAs described above. Hence, dsRNAs having a sequence of at least 19, 20, or more contiguous nucleotides derived from any one of the sequences of any one of Tables 2-3, and differing in their ability to inhibit the expression of a universal target sequence by not more than about 5, 10, 15, 20, 25, or 30% inhibition from a dsRNA comprising the full sequence, are contemplated to be within the scope of the present invention.

In addition, the RNAs provided in Tables 2-3 identify a site(s) in a universal target sequence transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 19 contiguous nucleotides from any one of the sequences provided in any one of Tables 2-3 coupled to additional nucleotide sequences taken from the region contiguous to the selected sequence in a universal target sequence.

### III. Modified Universal iRNAs of the Invention

In certain embodiments, the universal iRNA of the invention e.g., a dsRNA, is un-modified, and does not comprise, e.g., chemical modifications or conjugations known in the art and described herein. In other embodiments, the universal iRNA of the invention, e.g., a dsRNA, is chemically modified to enhance stability or other beneficial characteristics. In certain embodiments of the invention, substantially all of the nucleotides of a universal iRNA of the invention are modified, i.e., not more than 5, 4, 3, 2, or 1 unmodified nucleotides are present in a strand of the iRNA. In other embodiments of the invention, all of the nucleotides of a universal iRNA are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothiotate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothiotate groups present in the agent.

Representative U.S. Patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. Patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Suitable RNA mimetics are contemplated for use in iRNAs provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as O—P(O)(OH)—OCH₂—.

Modified RNAs can also contain one or more substituted sugar moieties. The iRNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative US patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

A universal iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxythimidine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. Patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In some embodiments, an RNAi agent of the disclosure can also be modified to include one or more bicyclic sugar moieties. A “bicyclic sugar” is a furanosyl ring modified by a ring formed by the bridging of two carbons, whether adjacent or non-adjacent. A “bicyclic nucleoside” (“BNA”) is a nucleoside having a sugar moiety comprising a ring formed by bridging two carbons, whether adjacent or non-adjacent, of the sugar ring, thereby forming a bicyclic ring system. In certain embodiments, the bridge connects the 4′-carbon and the 2′-carbon of the sugar ring, optionally, via the 2′-acyclic oxygen atom. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33 (1): 439-447; Mook, O R. et al., (2007) Mol Canc Ther 6 (3): 833-843; Grunweller, A. et al., (2003) Nucleic Acids Research 31 (12): 3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge.

A locked nucleoside can be represented by the structure (omitting stereochemistry),

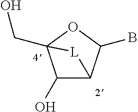

wherein B is a nucleobase or modified nucleobase and L is the linking group that joins the 2′-carbon to the 4′-carbon of the ribose ring. Examples of such 4′ to 2′ bridged bicyclic nucleosides, include but are not limited to 4′-(CH₂)—O-2′ (LNA); 4′-(CH₂)—S-2′; 4′-(CH₂)₂-O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C₁-C₁₂ alkyl, or a nitrogen protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and U.S. Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An iRNA of the invention can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge (i.e., L in the preceding structure). In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, U.S. Patent Publication No. 2013/0190383; and PCT publication WO 2013/036868, the entire contents of each of which are hereby incorporated herein by reference.

In some embodiments, an iRNA of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

In certain embodiments, the compositions and methods of the disclosure include a vinyl phosphonate (VP) modification of an RNAi agent as described herein. In exemplary embodiments, a 5′ vinyl phosphonate modified nucleotide of the disclosure has the structure:

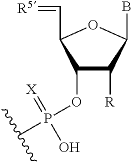

wherein X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphonate modifications are also contemplated for the compositions and methods of the instant disclosure. An exemplary vinyl phosphonate structure includes the preceding structure, where R5′ is ═C(H)—OP(O)(OH)₂ and the double bond between the C5′ carbon and R5′ is in the E or Z orientation (e.g., E orientation).

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3′-phosphate, inverted 2′-deoxy-modified ribonucleotide, such as inverted dT (idT), inverted dA (idA), and inverted abasic 2′-deoxyribonucleotide (iAb) and others. Disclosure of this modification can be found in WO 2011/005861.

In one example, the 3′ or 5′ terminal end of a oligonucleotide is linked to an inverted 2′-deoxy-modified ribonucleotide, such as inverted dT (idT), inverted dA (idA), or a inverted abasic 2′-deoxyribonucleotide (iAb). In one particular example, the inverted 2′-deoxy-modified ribonucleotide is linked to the 3′ end of an oligonucleotide, such as the 3′-end of a sense strand described herein, where the linking is via a 3′-3′ phosphodiester linkage or a 3′-3′-phosphorothioate linkage.

In another example, the 3′-end of a sense strand is linked via a 3′-3′-phosphorothioate linkage to an inverted abasic ribonucleotide (iAb). In another example, the 3′-end of a sense strand is linked via a 3′-3′-phosphorothioate linkage to an inverted dA (idA).

In one particular example, the inverted 2′-deoxy-modified ribonucleotide is linked to the 3′ end of an oligonucleotide, such as the 3′-end of a sense strand described herein, where the linking is via a 3′-3′ phosphodiester linkage or a 3′-3′-phosphorothioate linkage.

In another example, the 3′-terminal nucleotides of a sense strand is an inverted dA (idA) and is linked to the preceding nucleotide via a 3′-3′-linkage (e.g., 3′-3′-phosphorothioate linkage).

Other modifications of the nucleotides of an iRNA of the invention include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

A. Modified iRNAs Comprising Motifs of the Invention

In certain aspects of the invention, the double stranded RNA agents of the invention include agents with chemical modifications as disclosed, for example, in WO2013/075035, the entire contents of each of which are incorporated herein by reference. As shown herein and in WO2013/075035, one or more motifs of three identical modifications on three consecutive nucleotides may be introduced into a sense strand or antisense strand of a dsRNAi agent, particularly at or near the cleavage site. In some embodiments, the sense strand and antisense strand of the dsRNAi agent may otherwise be completely modified. The introduction of these motifs interrupts the modification pattern, if present, of the sense or antisense strand. The dsRNAi agent may be optionally conjugated with a GalNAc derivative ligand, for instance on the sense strand.

More specifically, when the sense strand and antisense strand of the double stranded RNA agent are completely modified to have one or more motifs of three identical modifications on three consecutive nucleotides at or near the cleavage site of at least one strand of a dsRNAi agent, the gene silencing activity of the dsRNAi agent was observed.

Accordingly, the invention provides double stranded RNA agents capable of inhibiting the expression of a universal target sequence in vivo. The RNAi agent comprises a sense strand and an antisense strand. Each strand of the RNAi agent may be, for example, 17-30 nucleotides in length, 25-30 nucleotides in length, 27-30 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as “dsRNAi agent.” The duplex region of a dsRNAi agent may be, for example, the duplex region can be 27-30 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 19, 20, 21, 22, 23, 24, 25, 26, and 27 nucleotides in length.

In certain embodiments, the dsRNAi agent may contain one or more overhang regions or capping groups at the 3′-end, 5′-end, or both ends of one or both strands. The overhang can be, independently, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In certain embodiments, the overhang regions can include extended overhang regions as provided above. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In certain embodiments, the nucleotides in the overhang region of the dsRNAi agent can each independently be a modified or unmodified nucleotide including, but no limited to 2′-sugar modified, such as, 2′-F, 2′-O-methyl, thymidine (T), 2-O-methoxyethyl-5-methyluridine (Teo), 2-O-methoxyethyladenosine (Aeo), 2-O-methoxyethyl-5-methylcytidine (m5Ceo), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand, or both strands of the dsRNAi agent may be phosphorylated. In some embodiments, the overhang region(s) contains two nucleotides having a phosphorothioate between the two nucleotides, where the two nucleotides can be the same or different. In some embodiments, the overhang is present at the 3′-end of the sense strand, antisense strand, or both strands. In some embodiments, this 3′-overhang is present in the antisense strand. In some embodiments, this 3′-overhang is present in the sense strand.

The dsRNAi agent may contain only a single overhang, which can strengthen the interference activity of the RNAi, without affecting its overall stability. For example, the single-stranded overhang may be located at the 3′-end of the sense strand or, alternatively, at the 3′-end of the antisense strand. The RNAi may also have a blunt end, located at the 5′-end of the antisense strand (i.e., the 3′-end of the sense strand) or vice versa. Generally, the antisense strand of the dsRNAi agent has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In certain embodiments, the dsRNAi agent is a double blunt-ended of 19 nucleotides in length, wherein the sense strand contains at least one motif of three 2′-F modifications on three consecutive nucleotides at positions 7, 8, 9 from the 5′-end. The antisense strand contains at least one motif of three 2′-O-methyl modifications on three consecutive nucleotides at positions 11, 12, and 13 from the 5′-end.

In other embodiments, the dsRNAi agent is a double blunt-ended of 20 nucleotides in length, wherein the sense strand contains at least one motif of three 2′-F modifications on three consecutive nucleotides at positions 8, 9, and 10 from the 5′-end. The antisense strand contains at least one motif of three 2′-O-methyl modifications on three consecutive nucleotides at positions 11, 12, and 13 from the 5′-end.

In yet other embodiments, the dsRNAi agent is a double blunt-ended of 21 nucleotides in length, wherein the sense strand contains at least one motif of three 2′-F modifications on three consecutive nucleotides at positions 9, 10, and 11 from the 5′-end. The antisense strand contains at least one motif of three 2′-O-methyl modifications on three consecutive nucleotides at positions 11, 12, and 13 from the 5′-end.

In certain embodiments, the dsRNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′-end; the antisense strand contains at least one motif of three 2′-O-methyl modifications on three consecutive nucleotides at positions 11, 12, and 13 from the 5′-end, wherein one end of the RNAi agent is blunt, while the other end comprises a 2 nucleotide overhang. In one embodiment, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In certain embodiments, every nucleotide in the sense strand and the antisense strand of the dsRNAi agent, including the nucleotides that are part of the motifs are modified nucleotides. In certain embodiments each residue is independently modified with a 2′-O-methyl or 3′-fluoro, e.g., in an alternating motif. Optionally, the dsRNAi agent further comprises a ligand (such as, GalNAc₃).

In certain embodiments, the dsRNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3 ‘terminal nucleotide of antisense strand is unpaired with sense strand, and up to 6 consecutive 3’ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce universal target site expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In certain embodiments, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce universal target site expression when the RNAi agent is introduced into a mammalian cell, and wherein Dicer cleavage of the dsRNAi agent results in an siRNA comprising the 3′-end of the second strand, thereby reducing expression of the universal target site in the mammal. Optionally, the dsRNAi agent further comprises a ligand.

In certain embodiments, the sense strand of the dsRNAi agent contains at least one motif of three identical modifications on three consecutive nucleotides, where one of the motifs occurs at the cleavage site in the sense strand.

In certain embodiments, the antisense strand of the dsRNAi agent can also contain at least one motif of three identical modifications on three consecutive nucleotides, where one of the motifs occurs at or near the cleavage site in the antisense strand.

For a dsRNAi agent having a duplex region of 19-23 nucleotides in length, the cleavage site of the antisense strand is typically around the 10, 11, and 12 positions from the 5′-end. Thus the motifs of three identical modifications may occur at the 9, 10, 11 positions; the 10, 11, 12 positions; the 11, 12, 13 positions; the 12, 13, 14 positions; or the 13, 14, 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the dsRNAi agent from the 5′-end.

The sense strand of the dsRNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In some embodiments, the sense strand of the dsRNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistries of the motifs are distinct from each other, and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the dsRNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In some embodiments, the wing modification on the sense strand or antisense strand of the dsRNAi agent typically does not include the first one or two terminal nucleotides at the 3′-end, 5′-end, or both ends of the strand.

In other embodiments, the wing modification on the sense strand or antisense strand of the dsRNAi agent typically does not include the first one or two paired nucleotides within the duplex region at the 3′-end, 5′-end, or both ends of the strand.

When the sense strand and the antisense strand of the dsRNAi agent each contain at least one wing modification, the wing modifications may fall on the same end of the duplex region, and have an overlap of one, two, or three nucleotides.

When the sense strand and the antisense strand of the dsRNAi agent each contain at least two wing modifications, the sense strand and the antisense strand can be so aligned that two modifications each from one strand fall on one end of the duplex region, having an overlap of one, two, or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In some embodiments, every nucleotide in the sense strand and antisense strand of the dsRNAi agent, including the nucleotides that are part of the motifs, may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′-hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′- or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′-end or ends can be phosphorylated.

It may be possible, e.g., to enhance stability, to include particular bases in overhangs, or to include modified nucleotides or nucleotide surrogates, in single strand overhangs, e.g., in a 5′- or 3′-overhang, or in both. For example, it can be desirable to include purine nucleotides in overhangs. In some embodiments all or some of the bases in a 3′- or 5′-overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, CRN, CET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others. In certain embodiments, the Nₐ or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′ to 3′ of the strand and the alternating motif in the antisense strand may start with “BABABA” from 5′ to 3′ of the strand within the duplex region. As another example, the alternating motif in the sense strand may start with “AABBAABB” from 5′ to 3′ of the strand and the alternating motif in the antisense strand may start with “BBAABBAA” from 5′ to 3′ of the strand within the duplex region, so that there is a complete or partial shift of the modification patterns between the sense strand and the antisense strand.

In some embodiments, the dsRNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand or antisense strand interrupts the initial modification pattern present in the sense strand or antisense strand. This interruption of the modification pattern of the sense or antisense strand by introducing one or more motifs of three identical modifications on three consecutive nucleotides to the sense or antisense strand may enhance the gene silencing activity against the universal target sequence.

In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ or N_{b} may be present or absent when there is a wing modification present.

The iRNA may further comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′ end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In one embodiment, the dsRNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In certain embodiments, the dsRNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In certain embodiments, the nucleotide at the 1 position within the duplex region from the 5′-end in the antisense strand is selected from A, dA, dU, U, and dT. Alternatively, at least one of the first 1, 2, or 3 base pair within the duplex region from the 5′-end of the antisense strand is an AU base pair. For example, the first base pair within the duplex region from the 5′-end of the antisense strand is an AU base pair.

In other embodiments, the nucleotide at the 3′-end of the sense strand is deoxythimidine (dT) or the nucleotide at the 3′-end of the antisense strand is deoxythimidine (dT). For example, there is a short sequence of deoxythimidine nucleotides, for example, two dT nucleotides on the 3′-end of the sense, antisense strand, or both strands.

In certain embodiments, the sense strand sequence may be represented by formula (I):

(I)
5′ nₚ-Nₐ-(XXX)ᵢ-N_{b}-YYY-N_{b}-(ZZZ)ⱼ-Nₐ-n_{q} 3′

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein N_{b} and Y do not have the same modification; and
XXX, YYY, and ZZZ each independently represent one motif of three identical modifications on three consecutive nucleotides. In one embodiment, YYY is all 2′-F modified nucleotides.

In some embodiments, the Nₐ or N_{b} comprises modifications of alternating pattern.

In some embodiments, the YYY motif occurs at or near the cleavage site of the sense strand. For example, when the dsRNAi agent has a duplex region of 17-23 nucleotides in length, the YYY motif can occur at or the vicinity of the cleavage site (e.g.: can occur at positions 6, 7, 8; 7, 8, 9; 8, 9, 10; 9, 10, 11; 10, 11, 12; or 11, 12, 13) of the sense strand, the count starting from the first nucleotide, from the 5′-end; or optionally, the count starting at the first paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′ nₚ-Nₐ-YYY-N_{b}-ZZZ-Nₐ-n_{q} 3′;
(Ic)
5′ nₚ-Nₐ-XXX-N_{b}-YYY-Nₐ-n_{q} 3′;
or
(Id)
5′ nₚ-Nₐ-XXX-N_{b}-YYY-N_{b}-ZZZ-Nₐ-n_{q} 3′.

When the sense strand is represented by formula (Ib), N_{b} represents an oligonucleotide sequence comprising 0-10, 0-7, 0-5, 0-4, 0-2, or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2, or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2, or 0 modified nucleotides. In one embodiment, N_{b} is 0, 1, 2, 3, 4, 5, or 6 Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′ nₚ-Nₐ-YYY-Nₐ-n_{q} 3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′ n_{q′}-Nₐ′-(Z′Z′Z′)ₖ-N_{b}′-Y′Y′Y′-N_{b}′-(X′X′X′)ₗ-N′ₐ-
nₚ′ 3′

wherein:
k and l are each independently 0 or 1;
p′ and q′ are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}′ and Y′ do not have the same modification; and
X′X′X′, Y′Y′Y′, and Z′Z′Z′ each independently represent one motif of three identical modifications on three consecutive nucleotides.

In some embodiments, the Nₐ′ or N_{b}′ comprises modifications of alternating pattern.

The Y′Y′Y′ motif occurs at or near the cleavage site of the antisense strand. For example, when the dsRNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense strand, with the count starting from the first nucleotide, from the 5′-end; or optionally, the count starting at the first paired nucleotide within the duplex region, from the 5′-end. In one embodiment, the Y′Y′Y′ motif occurs at positions 11, 12, 13.

In certain embodiments, Y′Y′Y′ motif is all 2′-OMe modified nucleotides.

In certain embodiments, k is 1 and l is 0, or k is 0 and l is 1, or both k and l are 1.

The antisense strand can therefore be represented by the following formulas:

(IIb)
5′ n_{q′}-Nₐ′-Z′Z′Z′-N_{b}′-Y′Y′Y′-Nₐ′-n_{p′} 3′;
(IIc)
5′ n_{q′}-Nₐ′-Y′Y′Y′-N_{b}′-X′X′X′-n_{p′} 3′;
or
(IId)
5′ n_{q′}-Nₐ′-Z′Z′Z′-N_{b}′-Y′Y′Y′-Nb′-X′X′X′-Nₐ′-n_{p′} 3′.

When the antisense strand is represented by formula (IIb), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides. In one embodiment, N_{b} is 0, 1, 2, 3, 4, 5, or 6.

In other embodiments, k is 0 and 1 is 0 and the antisense strand may be represented by the formula:

(Ia)
5′ n_{p′}-N_{a′}-Y′Y′Y′-N_{a′}-n_{q′} 3′.

When the antisense strand is represented as formula (IIa), each Nₐ′ independently represents an oligonucleotide sequence comprising 2-20, 2-15, or 2-10 modified nucleotides. Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′, and Z′, in particular, may represent a 2′-O-methyl modification or a 2′-fluoro modification.

In some embodiments, the sense strand of the dsRNAi agent may contain YYY motif occurring at 9, 10, and 11 positions of the strand when the duplex region is 21 nt, the count starting from the first nucleotide from the 5′-end, or optionally, the count starting at the first paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In some embodiments the antisense strand may contain Y′Y′Y′ motif occurring at positions 11, 12, 13 of the strand, the count starting from the first nucleotide from the 5′-end, or optionally, the count starting at the first paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the dsRNAi agents for use in the methods of the invention may comprise a sense strand and an antisense strand, each strand having 14 to 30 nucleotides, the iRNA duplex represented by formula (III):

(III)
sense:
5′ nₚ-Nₐ-(XXX)ᵢ-N_{b}-YYY-N_{b}-(ZZZ)ⱼ-Nₐ-n_{q} 3′
antisense:
3′ nₚ^{′}-Nₐ^{′}-(X′X′X′)ₖ-N_{b}^{′}-Y′Y′Y′-N_{b}^{′}-(Z′Z′Z′)ₗ-Nₐ^{′}-
n_{q}^{′} 5′

wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and l is 0; k is 0 and l is 1; or both k and l are 0; or both k and l are 1.

Exemplary combinations of the sense strand and antisense strand forming an iRNA duplex include the formulas below:

(IIIa)
5′ nₚ-Nₐ-YYY-Nₐ-n_{q} 3′
3′ nₚ^{′}-Nₐ^{′}-Y′Y′Y′-Nₐ^{′}n_{q}^{′} 5′
(IIIb)
5′ nₚ-Nₐ-YYY-N_{b}-ZZZ-Nₐ-n_{q} 3′
3′ nₚ^{′}-Nₐ^{′}-Y′Y′Y′-N_{b}^{′}-Z′Z′Z′-Nₐ^{′} n_{q}^{′} 5′
(IIIc)
5′ nₚ-Nₐ-XXX-N_{b}-YYY-Nₐ-n_{q} 3′
3′ nₚ^{′}-Nₐ^{′}-X′X′X′-N_{b}^{′}-Y′Y′Y′-Nₐ^{′}-n_{q}^{′} 5′
(IIId)
5′ nₚ-Nₐ-XXX-N_{b}-YYY-N_{b}-ZZZ-Nₐ-n_{q} 3′
3′ nₚ^{′}-Nₐ^{′}-X′X′X′-N_{b}-Y′Y′Y′-N_{b}^{′}-Z′Z′Z′-Nₐ-n_{q}^{′} 5′

When the dsRNAi agent is represented by formula (IIIa), each Nₐ independently represents an oligonucleotide sequence comprising 2-20, 2-15, or 2-10 modified nucleotides.

When the dsRNAi agent is represented by formula (IIIb), each N_{b} independently represents an oligonucleotide sequence comprising 1-10, 1-7, 1-5, or 1-4 modified nucleotides. Each Nₐ independently represents an oligonucleotide sequence comprising 2-20, 2-15, or 2-10 modified nucleotides.

When the dsRNAi agent is represented as formula (IIIc), each N_{b}, N_{b}′ independently represents an oligonucleotide sequence comprising 0-10, 0-7, 0-10, 0-7, 0-5, 0-4, 0-2, or 0 modified nucleotides. Each Nₐ independently represents an oligonucleotide sequence comprising 2-20, 2-15, or 2-10 modified nucleotides.

When the dsRNAi agent is represented as formula (IIId), each N_{b}, N_{b}′ independently represents an oligonucleotide sequence comprising 0-10, 0-7, 0-10, 0-7, 0-5, 0-4, 0-2, or 0 modified nucleotides. Each Nₐ, Nₐ′ independently represents an oligonucleotide sequence comprising 2-20, 2-15, or 2-10 modified nucleotides. Each of Nₐ, Nₐ′, No, and N_{b}′ independently comprises modifications of alternating pattern.

Each of X, Y, and Z in formulas (III), (IIIa), (IIIb), (IIIc), and (IIId) may be the same or different from each other.

When the dsRNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the dsRNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the dsRNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In certain embodiments, the modification on the Y nucleotide is different than the modification on the Y′ nucleotide, the modification on the Z nucleotide is different than the modification on the Z′ nucleotide, or the modification on the X nucleotide is different than the modification on the X′ nucleotide.

In certain embodiments, when the dsRNAi agent is represented by formula (IIId), the Nₐ modifications are 2′-O-methyl or 2′-fluoro modifications. In other embodiments, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more GalNAc derivatives attached through a bivalent or trivalent branched linker (described below). In other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, when the dsRNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

As described in more detail below, the iRNA that contains conjugations of one or more carbohydrate moieties to an iRNA can optimize one or more properties of the iRNA. In many cases, the carbohydrate moiety will be attached to a modified subunit of the iRNA. For example, the ribose sugar of one or more ribonucleotide subunits of a iRNA can be replaced with another moiety, e.g., a non-carbohydrate (such as, cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” such as, two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide, or polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The iRNA may be conjugated to a ligand via a carrier, wherein the carrier can be cyclic group or acyclic group. In one embodiment, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a serinol backbone or diethanolamine backbone.

### B. Thermally Destabilizing Modifications

In certain embodiments, a dsRNA molecule can be optimized for RNA interference by incorporating thermally destabilizing modifications in the seed region of the antisense strand. As used herein “seed region” means at positions 2-9 of the 5′-end of the referenced strand or at positions 2-8 of the 5′-end of the refrenced strand. For example, thermally destabilizing modifications can be incorporated in the seed region of the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celcius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, such as, positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

An iRNA agent comprises a sense strand and an antisense strand, each strand having 14 to 40 nucleotides. The RNAi agent may be represented by formula (L):

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA, 2′O—CH₂C(O)N(Me)H) modification.

C1 is a thermally destabilizing nucleotide placed at a site opposite to the seed region of the antisense strand (i.e., at positions 2-8 of the 5′-end of the antisense strand, or at positions 2-9 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA), or 2′-5′-linked ribonucleotides (“3′-RNA”). In one embodiment, C1 has thermally destabilizing modification selected from the group consisting of: i) mismatch with the opposing nucleotide in the antisense strand; ii) abasic modification selected from the group consisting of:

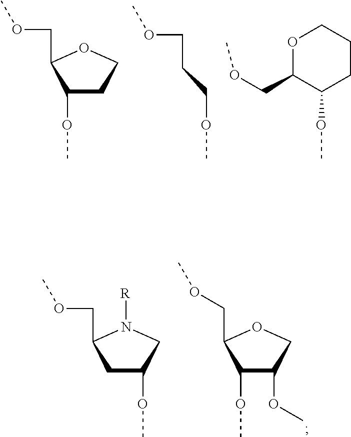

and iii) sugar modification selected from the group consisting of:

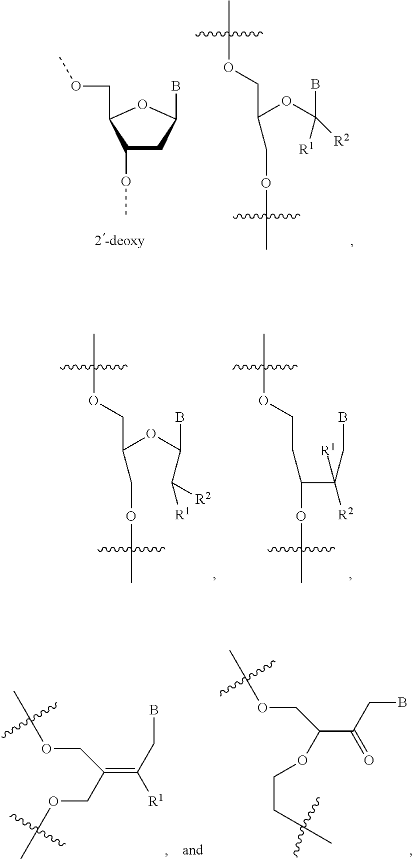

wherein B is a modified or unmodified nucleobase, R¹ and R² independently are H, halogen, OR₃, or alkyl; and R³ is H, alkyl, cycloalkyl, aryl, aralkyl, heteroaryl or sugar. In one embodiment, the thermally destabilizing modification in C₁ is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

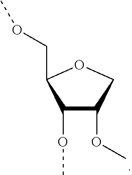

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.
n¹, n³, and q¹ are independently 4 to 15 nucleotides in length.
n⁵, q³, and q⁷ are independently 1-6 nucleotide(s) in length.
n⁴, q², and q⁶ are independently 1-3 nucleotide(s) in length; alternatively, n⁴ is 0.
q⁵ is independently 0-10 nucleotide(s) in length.
n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C₁ is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The RNAi agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

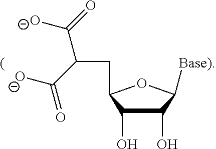

When the 5′-end phosphorus-containing group is 5′-end vinylphosphonate (5′-VP), the 5′-VP can be either 5′-E-VP isomer

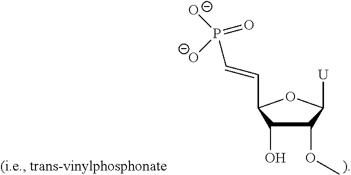

5′-Z-VP isomer

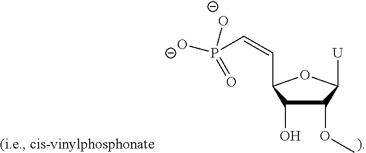

or mixtures thereof.

In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z-VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof), and a targeting ligand.

In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end); and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a deoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and deoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);

wherein the RNAi agents have a two nucleotide overhang at the 3′-end of the antisense strand, and a blunt end at the 5′-end of the antisense strand.

In certain embodiments, the iRNA for use in the methods of the invention is an agent selected from the agents listed in any one of Tables 2-3. These agents may further comprise a ligand.

### IV. REVERSIR Compounds of the Invention

The present invention also provides REVERSIR compounds which abrogate the activity of the universal dsRNA agents of the invention. The design, synthesis, and suitable modifications of REVERSIR compounds are disclosed in WO 2016/100716, WO 2019/036612, and U.S. 2017/369872, the entire contents of each of which are incorporated herein by reference.

Generally, the REVERSIR compounds of the invention are single stranded oligonucleotides (oligomers) 6-30 nucleotides in length. The nucleotide sequence of the oligonucleotides may be at least about 90%, e.g., 90%, 91%, 92%, 93%, 94%, 95%, 96%, 97%, 98%, 99%, or 100% complementary to any one of the antisense strand nucleotide sequences in Table 2 or 3.

Exemplary REVERSIR compounds of the invention are presented herein in Table 4.

In certain embodiments, the REVERSIR compounds are chemically modified oligomeric compounds, compared to naturally occurring oligomers, such as DNA or RNA.

Thus, in certain embodiment, the REVERSIR compounds of the invention comprise a least one modified nucleotide, i.e., at least one modified monomer. In other embodiments, substantially all of the nucleotides of the oligonucleotide are modified nucleotides. In still other embodiment, all of the nucleotides of the oligonucleotide are modified nucleotides.

In certain such embodiments, the REVERSIR compounds of the invention comprise one or more high affinity monomer. In certain embodiments, such high-affinity monomer is selected from monomers (e.g., nucleosides and nucleotides) comprising 2′-modified sugars, including, but not limited to: BNA's and monomers (e.g., nucleosides and nucleotides) with 2′-substituents such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rm)(Rn), or O—CH₂—C(═O)—N(Rm)(Rn), where each Rm and Rn is, independently, H or substituted or unsubstituted C₁-C₁₀ alkyl.

In certain embodiments, the REVERSIR compounds of the invention comprise one or more β-D-Methyleneoxy (4′-CH₂—O-2′) LNA monomers.

In certain embodiments, the REVERSIR compounds of the invention comprise one or more α-D-Methyleneoxy (4′-CH₂—O-2′) LNA monomers.

In certain embodiments, the REVERSIR compounds of the invention comprise one or more (S)-cEt monomers.

In certain embodiments, the REVERSIR compounds of the invention comprise one or more high affinity monomers provided that the compound does not comprise a nucleotide comprising a 2′-O(CH₂)ₙH, wherein n is one to six.

In certain embodiments, the REVERSIR compounds of the invention comprise one or more high affinity monomer provided that the compound does not comprise a nucleotide comprising a 2′-OCH₃ or a 2′-O(CH₂)₂OCH₃.

In certain embodiments, the REVERSIR compounds of the invention comprise one or more (e.g., 1, 2, 3, 4, 5, 6, 7, 8, 9, 10, 11, 12, 13, 14, 15 or more) high affinity monomer provided that the compound does not comprise a α-L-Methyleneoxy (4′-CH₂—O-2′) LNA.

In certain embodiments, the REVERSIR compounds of the invention comprise one or more high affinity monomer provided that the compound does not comprise a β-D-Methyleneoxy (4′-CH₂—O-2′) LNA.

In certain embodiments, the REVERSIR compounds of the invention comprise one or more high affinity monomer provided that the compound does not comprise a α-L-Methyleneoxy (4′-CH₂—O-2′) LNA or β-D-Methyleneoxy (4′-CH₂—O-2′) LNA.

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside or internucleotide backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

A REVERSIR compound as described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2 (thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2 (thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2 (thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo) thymine, 2-pyridinone, 5-nitroindole, 3-nitropyrrole, 6-(aza)pyrimidine, 2-(amino) purine, 2,6-(diamino) purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

In some embodiments, the REVERSIR compounds of the invention comprise at least one (e.g., 1, 2, 3, 4, 5, 6, 7, 8, 9, or 10) G-clamp nucleobase selected from the following:

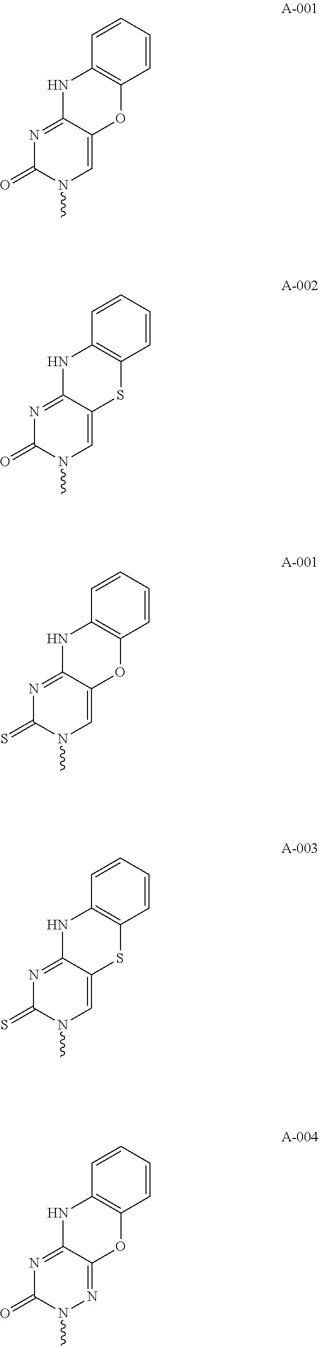

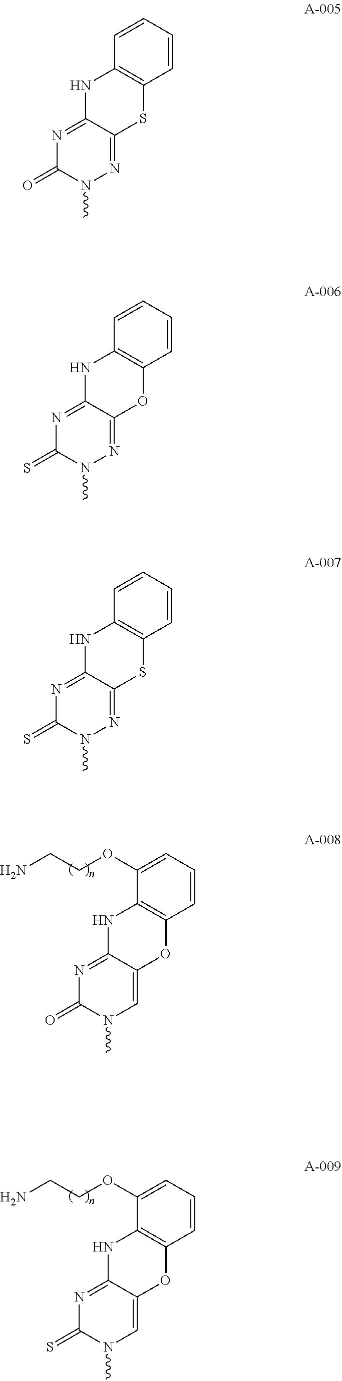

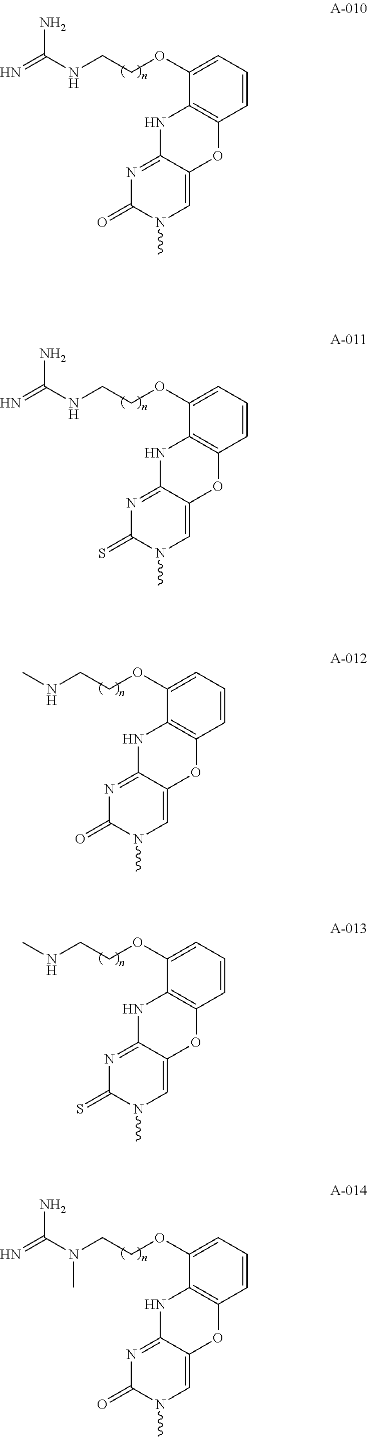

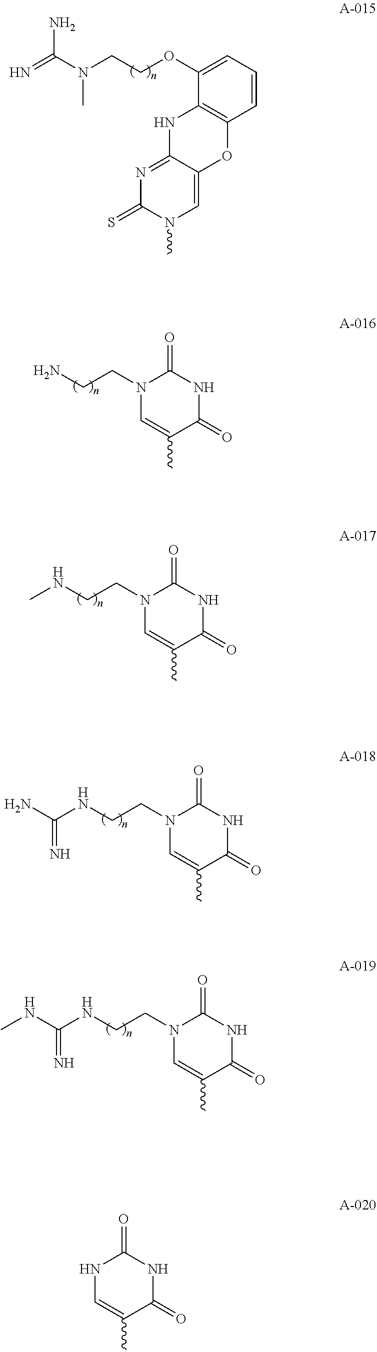

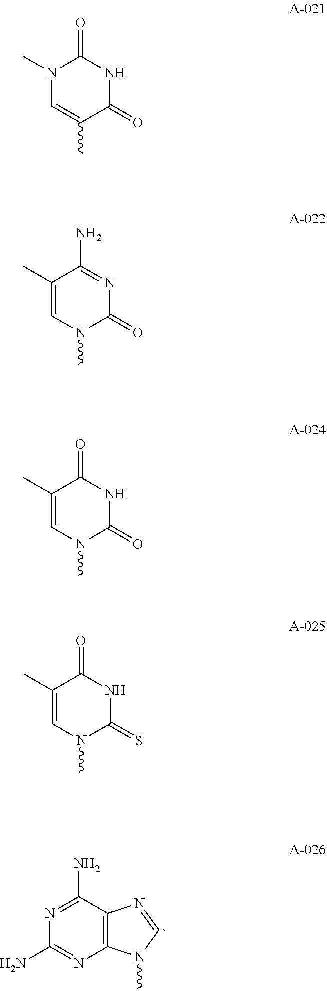

where n is 0, 1, 2, 3, 4, 5 or 6.

The REVERSIR compounds provided herein can comprise one or more monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, compounds comprise one or more monomers that are LNA.

In some embodiments of a locked nucleic acid, the 2′ position of furnaosyl is connected to the 4′ position by a linker selected independently from —[C(R1)(R2)]ₙ-, —[C(R1)(R2)]ₙ-O—, —[C(R1)(R2)]ₙ-N(R1)-, —[C(R1)(R2)]ₙ-N(R1)-O—, —[C(R1R2)]ₙ-O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R¹ and R² is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In one embodiment, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R=H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, a REVERSIR compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The REVERSIR compounds of the invention can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. REVERSIR compounds can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

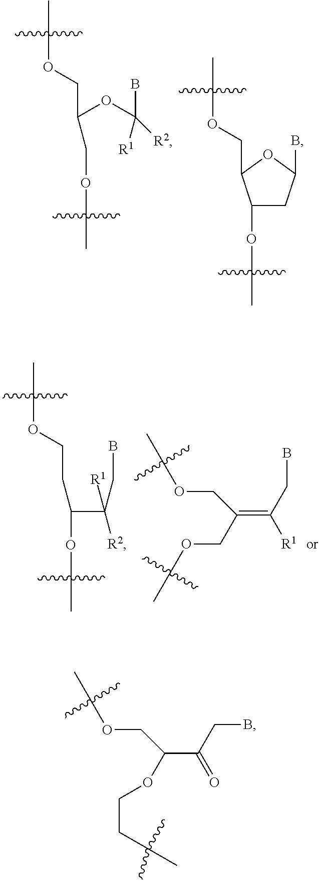

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

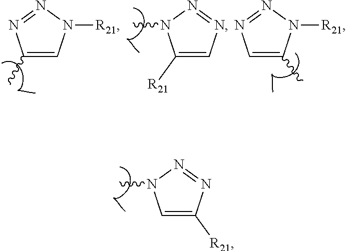

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

In certain embodiments, LNA's include bicyclic nucleotide having the formula:

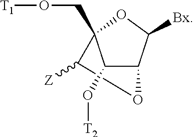

wherein:
Bx is a heterocyclic base moiety;
T1 is H or a hydroxyl protecting group;
T2 is H, a hydroxyl protecting group or a reactive phosphorus group;
Z is C1-C6 alkyl, C2-C6 alkenyl, C2-C6 alkynyl, substituted C1-C6 alkyl, substituted C2-C6 alkenyl, substituted C2-C6 alkynyl, acyl, substituted acyl, or substituted amide.

In one embodiment, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C1-C6 alkyl, or substituted C1-C6 alkyl and X is O or NJ1.

In certain embodiments, the Z group is C1-C6 alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is C1-C6 alkyl substituted with one or more Xx, wherein each Xx is independently halo (e.g., fluoro), hydroxyl, alkoxy (e.g., CH3O—), substituted alkoxy or azido.

In certain embodiments, the Z group is —CH2Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is —CH2Xx, wherein Xx is halo (e.g., fluoro), hydroxyl, alkoxy (e.g., CH3O—) or azido.

In certain such embodiments, the Z group is in the (R)-configuration:

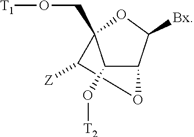

In certain such embodiments, the Z group is in the(S)-configuration:

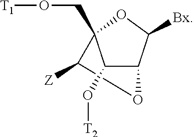

In certain embodiments, each T1 and T2 is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl) xanthine-9-yl (MOX). In certain embodiments, T1 is a hydroxyl protecting group selected from acetyl, benzyl, t-butyldimethylsilyl, t-butyldiphenylsilyl and dimethoxytrityl wherein a more preferred hydroxyl protecting group is T1 is 4,4′-dimethoxytrityl.

In certain embodiments, T2 is a reactive phosphorus group wherein preferred reactive phosphorus groups include diisopropylcyanoethoxy phosphoramidite and H-phosphonate. In certain embodiments T1 is 4,4′-dimethoxytrityl and T2 is diisopropylcyanoethoxy phosphoramidite.

In certain embodiments, REVERSIR compounds have at least one monomer of the formula:

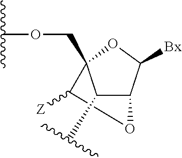

or of the formula:

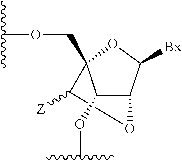

or of the formula:

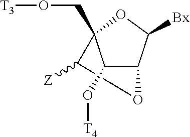

wherein
Bx is a heterocyclic base moiety;
T3 is H, a hydroxyl protecting group, a linked conjugate group or an internucleoside linking group attached to a nucleoside, a nucleotide, an oligonucleoside, an oligonucleotide, a monomeric subunit or an oligomeric compound;
T4 is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T3 and T4 is an internucleoside linking group attached to a nucleoside, a nucleotide, an oligonucleoside, an oligonucleotide, a monomeric subunit or an oligomeric compound; and
Z is C1-C6 alkyl, C2-C6 alkenyl, C2-C6 alkynyl, substituted C1-C6 alkyl, substituted C2-C6 alkenyl, substituted C2-C6 alkynyl, acyl, substituted acyl, or substituted amide.

In one embodiment, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In one embodiment, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C1-C6 alkyl or substituted C1-C6 alkyl. In certain embodiments, each Z is, independently, C1-C6 alkyl or substituted C1-C6 alkyl. In certain embodiments, at least one Z is C1-C6 alkyl. In certain embodiments, each Z is, independently, C1-C6 alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C1-C6 alkyl. In certain embodiments, each Z is, independently, substituted C1-C6 alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C1-C6 alkoxy (e.g., at least one Z is C1-C6 alkyl substituted with one or more C1-C6 alkoxy). In another embodiment, each substituent group is, independently, C1-C6 alkoxy (e.g., each Z is, independently, C1-C6 alkyl substituted with one or more C1-C6 alkoxy).

In certain embodiments, at least one C1-C6 alkoxy substituent group is CH3O— (e.g., at least one Z is CH₃OCH₂—). In another embodiment, each C1-C6 alkoxy substituent group is CH3O— (e.g., each Z is CH₃OCH₂—).

In certain embodiments, at least one substituent group is halogen (e.g., at least one Z is C1-C6 alkyl substituted with one or more halogen). In certain embodiments, each substituent group is, independently, halogen (e.g., each Z is, independently, C1-C6 alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C1-C6 alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C1-C6 alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, at least one Z group is C1-C6 alkyl substituted with one or more Xx, wherein each Xx is, independently, halo (e.g., fluoro), hydroxyl, alkoxy (e.g., CH3O—) or azido. In certain embodiments, each Z group is, independently, C1-C6 alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, each Z group is, independently, C1-C6 alkyl substituted with one or more Xx, wherein each Xx is independently halo (e.g., fluoro), hydroxyl, alkoxy (e.g., CH3O—) or azido.

In certain embodiments, at least one Z group is —CH₂Xx, wherein Xx is OJ1, NJ1J2, SJ1, N³, OC(═X)J1, OC(═X)NJ1J2, NJ3C(═X)NJ1J2 or CN; wherein each J1, J2 and J3 is, independently, H or C1-C6 alkyl, and X is O, S or NJ1 In certain embodiments, at least one Z group is —CH₂Xx, wherein Xx is halo (e.g., fluoro), hydroxyl, alkoxy (e.g., CH3O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃.

In certain embodiments, the Z group of at least one monomer is in the (R)- configuration represented by the formula:

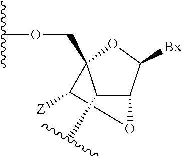

or the formula:

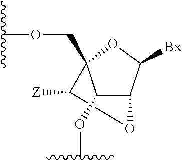

or the formula:

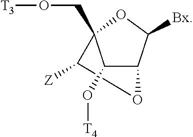

In certain embodiments, the Z group of each monomer of the formula is in the (R)- configuration.

In certain embodiments, the Z group of at least one monomer is in the (S)- configuration represented by the formula:

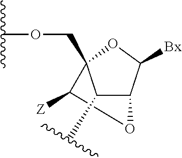

or the formula:

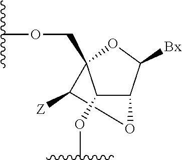

or the formula:

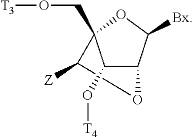

In certain embodiments, the Z group of each monomer of the formula is in the (S)-configuration.

In certain embodiments, T3 is H or a hydroxyl protecting group. In certain embodiments, T4 is H or a hydroxyl protecting group. In a further embodiment T3 is an internucleoside linking group attached to a nucleoside, a nucleotide or a monomeric subunit. In certain embodiments, T4 is an internucleoside linking group attached to a nucleoside, a nucleotide or a monomeric subunit. In certain embodiments, T3 is an internucleoside linking group attached to an oligonucleoside or an oligonucleotide. In certain embodiments, T4 is an internucleoside linking group attached to an oligonucleoside or an oligonucleotide. In certain embodiments, T3 is an internucleoside linking group attached to an oligomeric compound. In certain embodiments, T4 is an internucleoside linking group attached to an oligomeric compound. In certain embodiments, at least one of T3 and T4 comprises an internucleotide linking group selected from phosphodiester or phosphorothioate.

In certain embodiments, REVERSIR compounds have at least one region of at least two contiguous monomers of the formula:

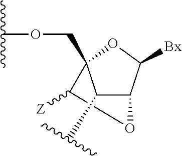

or of the formula:

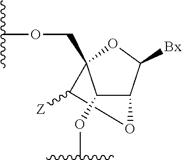

or of the formula:

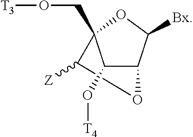

In certain such embodiments, LNAs include, but are not limited to, (A) α-L-Methyleneoxy (4′-CH2-O-2′) LNA, (B) β-D-Methyleneoxy (4′-CH2-O-2′) LNA, (C) Ethyleneoxy (4′-(CH2)2-O-2′) LNA, (D) Aminooxy (4′-CH2-O—N(R)-2′) LNA and (E) Oxyamino (4′-CH2-N(R)—O-2′) LNA, as depicted below:

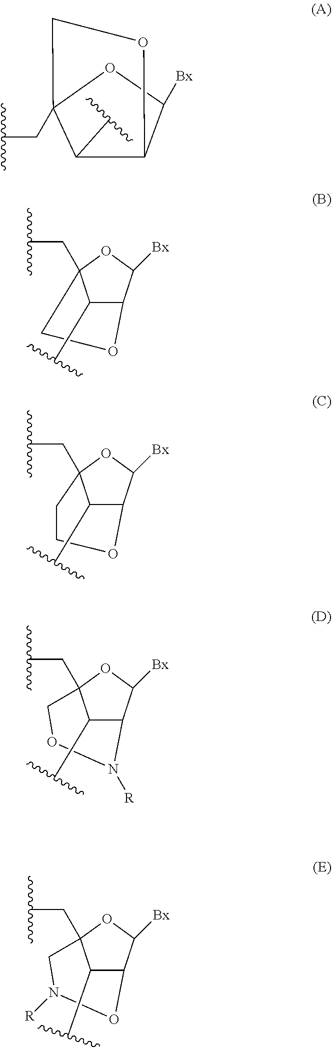

In certain embodiments, the REVERSIR compounds of the invention comprises at least two regions of at least two contiguous monomers of the above formula. In certain embodiments, the compound comprises a gapped oligomeric compound. In certain embodiments, the REVERSIR compounds of the invention comprises at least one region of from about 8 to about 14 contiguous β-D-2′-deoxyribofuranosyl nucleosides. In certain embodiments, the compound comprises at least one region of from about 9 to about 12 contiguous β-D-2′-deoxyribofuranosyl nucleosides.

In certain embodiments, the REVERSIR compound comprises at least one (e.g., 1, 2, 3, 4, 5, 6, 7, 8, 9, 10, 11, 12, 13, 14, 15 or more) S-cEt monomer of the formula:

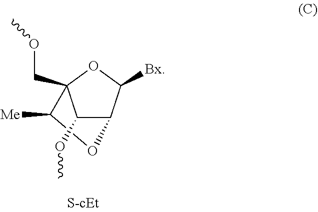

wherein Bx IS heterocyclic base moiety.

In some embodiments, the REVERSIR compounds of the invention comprises at least one (e.g., 1, 2, 3, 4, 5, 6, 7, 8, 9, 10, 11, 12, 13, 14, 15 or more) nucleotide selected from the following:

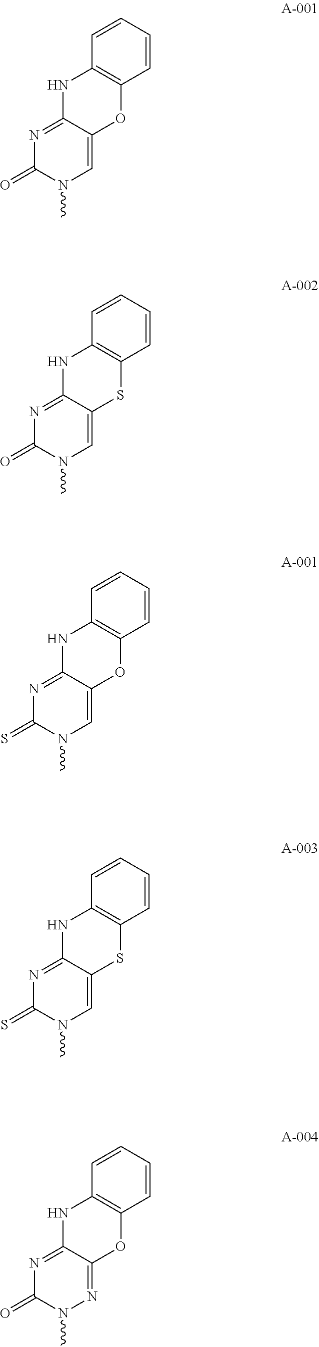

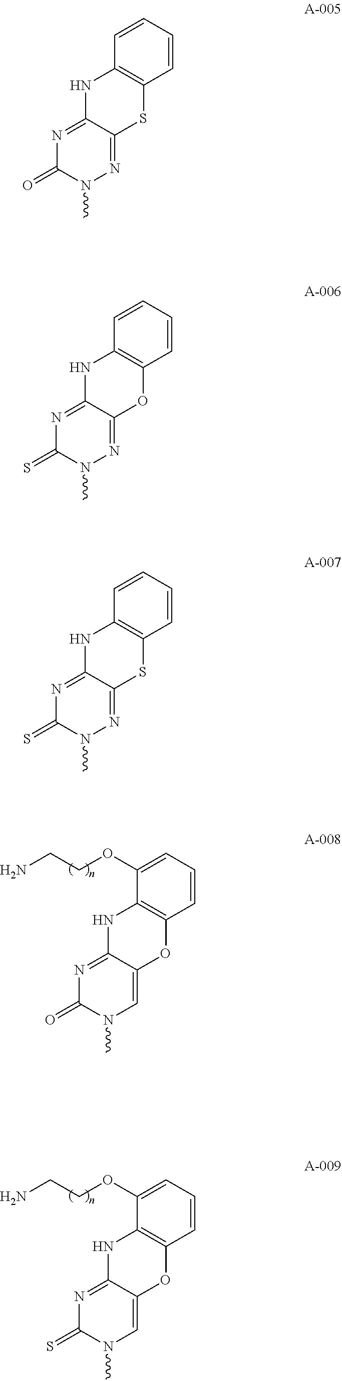

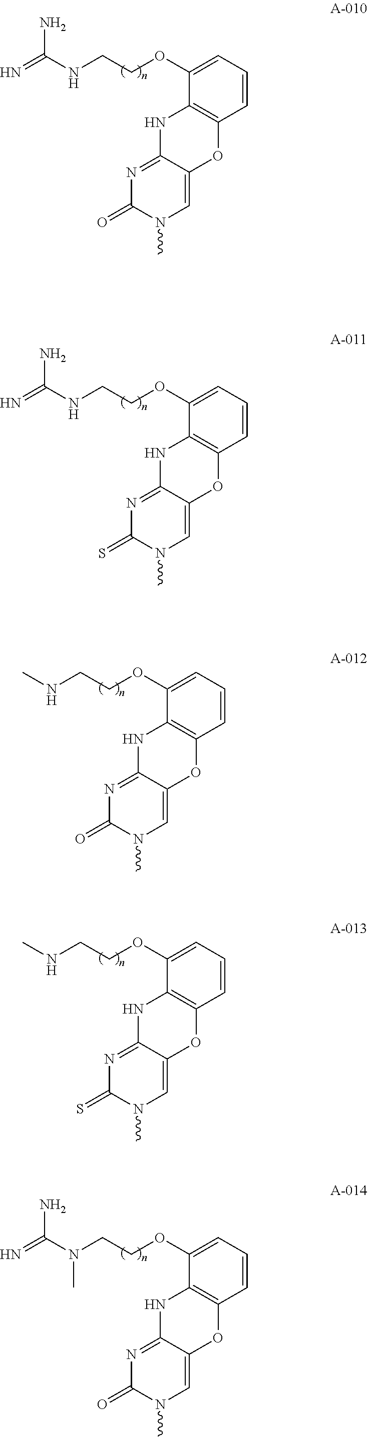

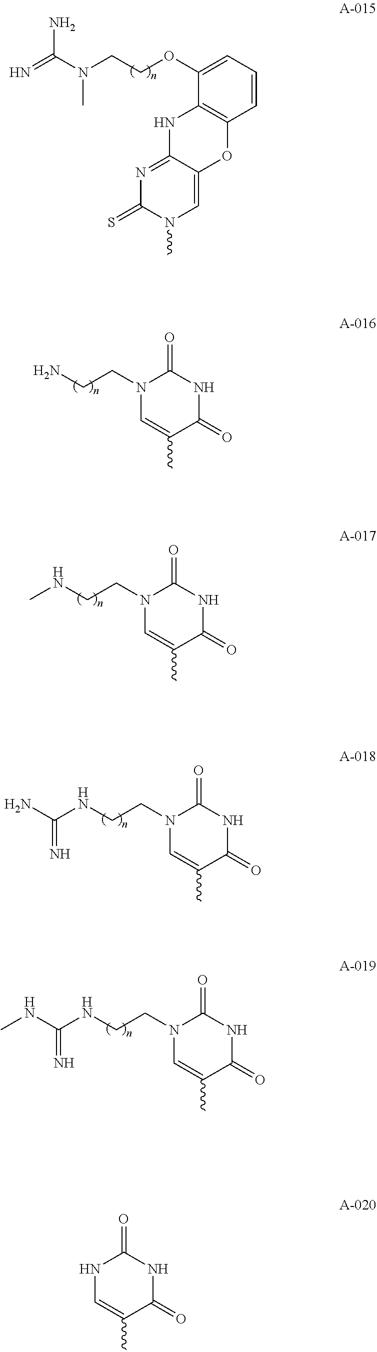

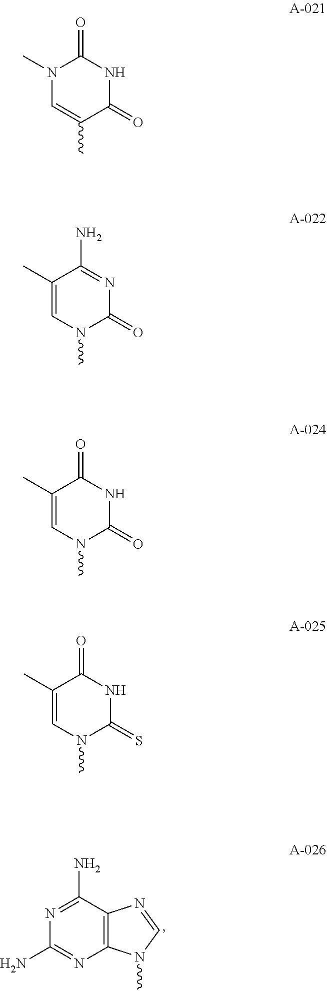

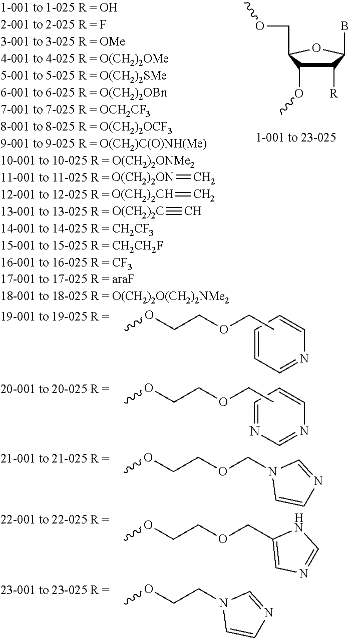

where B is A-001 to A-026 and n is 0-6 (e.g., 0, 1, 2, 3, 4, 5 or 6).

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside, nucleotide and nucleobase mimetics are well known to those skilled in the art.

In certain embodiments, the REVERSIR compounds of the invention comprise at least one monomer that is LNA and at least one G-clamp nucleobase. For example, the REVERSIR compound can comprise 1, 2, 3, 4, 5, 6, 7, 8, 9, 10, 11, 12, 13, 14, 15 or more monomers that are LNA 1, 2, 3, 4, 5, 6, 7, 8, 9, 10, 11, 12, 13, 14, 15 or more G-clamp nucleobases.

In some embodiments, the REVERSIR compounds of the invention comprise at least one (e.g., 1, 2, 3, 4, 5, 6, 7, 8, 9, 10, 11, 12, 13, 14, 15 or more) peptide nucleic acid monomer. In certain embodiments, the REVERSIR compound comprises at least one monomer that is LNA and at least one monomer that is PNA. For example, the REVERSIR compound can comprise 1, 2, 3, 4, 5, 6, 7, 8, 9, 10, 11, 12, 13, 14, 15 or more monomers that are LNA 1, 2, 3, 4, 5, 6, 7, 8, 9, 10, 11, 12, 13, 14, 15 or more monomers that are PNA.

In certain embodiments, the REVERSIR compounds of the invention comprise at least one PNA monomer and at least one G-clamp nucleobase. For example, the REVERSIR compound can comprise 1, 2, 3, 4, 5, 6, 7, 8, 9, 10, 11, 12, 13, 14, 15 or more PNA monomers and 1, 2, 3, 4, 5, 6, 7, 8, 9, 10, 11, 12, 13, 14, 15 or more G-clamp nucleobases.

In certain embodiments, the REVERSIR compounds of the invention comprise at least one LNA monomer, at least one PNA monomer and at least one G-clamp nucleobase. For example, the REVERSIR compound can comprise 1, 2, 3, 4, 5, 6, 7, 8, 9, 10, 11, 12, 13, 14, 15 or more LNA monomers; 1, 2, 3, 4, 5, 6, 7, 8, 9, 10, 11, 12, 13, 14, 15 or more PNA monomers and 1, 2, 3, 4, 5, 6, 7, 8, 9, 10, 11, 12, 13, 14, 15 or more G-clamp nucleobases.

Described herein are linking groups that link monomers (including, but not limited to, modified and unmodified nucleosides and nucleotides) together, thereby forming an oligomeric compound, i.e., a REVERSIR compound comprising an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the REVERSIR compounds of the invention comprise at least one (e.g., 1, 2, 3, 4, 5, 6, 7, 8, 9, 10, 11, 12, 13, 14, 15 or more and upto including all) modified or nonphosphodiester linkages. In one embodiment, the REVERSIR compounds of the invention comprise at least one (e.g., 1, 2, 3, 4, 5, 6, 7, 8, 9, 10, 11, 12, 13, 14, 15 or more and upto including all) phosphorothioate linkages.

In some embodiments, all internucleotide linkages in the reverser compounds are phosphorothioate (PS) internucleotide linkages. In certain embodiments, the REVERSIR compounds comprise at least one phosphorothioate (PS) internucleotide linkage, but not all internucleotide linkages in said REVERSIR compound are a phosphorothioate linkage. In other words, in some embodiments, less than 100% (e.g., 95%, 90%, 85%, 80%, 75%, 70%, 65%, 60%, 55%, 50%, 45%, 40% or fewer) of the internucleotide linkages are phosphorothioate linkages.

In some embodiments, the REVERSIR compounds comprise at least one phosphorothioate internucleotide linkage and at least one internucleoside or internucleotide linkage that is not a phosphorothioate. For example, the REVERSIR compounds comprise at least one phosphorothioate internucleotide linkage and at least one phosphodiester internucleotide linkage. In some embodiments, the non-phosphorothioate internucleotide linkage is between the terminus and the penultimate nucleotides.

In some embodiments, the internucleotide linkage between the nucleobase at the 3′-terminus of the REVERSIR compound and the rest of the REVERSIR compound is a phosphodiester linkage. In some embodiments, all internucleotide linkages in the REVERSIR compounds are phosphorothioate except for the internucleotide linkage between the nucleotide at the 3′-terminus of the REVERSIR compound and the rest of the REVERSIR compound.

REVERSIR compounds can also be constructed wherein the phosphate linker and the sugar are replaced by nuclease resistant nucleoside, nucleotide or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The REVERSIR compounds described herein may contain one or more asymmetric centers and thus give rise to enantiomers, diastereomers, and other stereoisomeric configurations that may be defined, in terms of absolute stereochemistry, as (R) or(S), such as for sugar anomers, or as (D) or (L) such as for amino acids et al. Included in the antisense compounds provided herein are all such possible isomers, as well as their racemic and optically pure forms.

Ends of the REVERSIR compounds of the invention may be modified. Such modifications can be at one end or both ends. For example, the 3′ and/or 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type compound.

Terminal modifications useful for modulating activity include modification of the 5′ end of compound with phosphate or phosphate analogs. In certain embodiments, the 5′ end of compound is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the compound comprises the modification

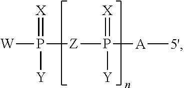

wherein W, X and Y are each independently selected from the group consisting of O, OR (R is hydrogen, alkyl, aryl), S, Se, BR₃ (R is hydrogen, alkyl, aryl), BH₃, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O) P—O—P(HO)(O)—O-5′); 5′-monothiophosphate (phosphorothioate; (HO)₂(S) P—O-5′); 5′-monodithiophosphate (phosphorodithioate; (HO)(HS)(S)P—O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}— 5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

In certain embodiments, the REVERSIR compounds of the invention are chimeric oligomeric compounds, i.e., chimeric oligonucleotides. In certain such embodiments, the chimeric oligonucleotides comprise differently modified nucleotides. In certain embodiments, chimeric oligonucleotides are mixed-backbone antisense oligonucleotides.

In general, a chimeric oligomeric compound will have modified nucleosides that can be in isolated positions or grouped together in regions that will define a particular motif. Any combination of modifications and/or mimetic groups can comprise a chimeric oligomeric compound as described herein.

In certain embodiments, chimeric oligomeric compounds typically comprise at least one region modified so as to confer increased resistance to nuclease degradation, increased cellular uptake, and/or increased binding affinity for the target nucleic acid. In certain embodiments, an additional region of the oligomeric compound may serve as a substrate for enzymes capable of cleaving RNA:DNA or RNA:RNA hybrids.

In certain embodiments, chimeric oligomeric compounds are gapmers. In certain such embodiments, a mixed-backbone oligomeric compound has one type of internucleotide linkages in one or both wings and a different type of internucleoside linkages in the gap. In certain such embodiments, the mixed-backbone oligonucleotide has phosphodiester linkages in the wings and phosphorothioate linkages in the gap. In certain embodiments in which the internucleotide linkages in a wing is different from the internucleotide linkages in the gap, the internucleotide linkage bridging that wing and the gap is the same as the internucleotide linkage in the wing. In certain embodiments in which the internucleotide linkages in a wing is different from the internucleotide linkages in the gap, the internucleotide linkage bridging that wing and the gap is the same as the internucleotide linkage in the gap.

In certain embodiments, the present invention provides REVERSIR compounds of any of a variety of ranges of lengths. In certain embodiments, the invention provides compounds consisting of X-Y linked oligonucleotides, where X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, the invention provides compounds comprising: 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 13-14, 13-15, 13-16, 13-17, 13-18, 13-19, 13-20, 13-21, 13-22, 13-23, 13-24, 13-25, 13-26, 13-27, 13-28, 13-29, 13-30, 14-15, 14-16, 14-17, 14-18, 14-19, 14-20, 14-21, 14-22, 14-23, 14-24, 14-25, 14-26, 14-27, 14-28, 14-29, 14-30, 15-16, 15-17, 15-18, 15-19, 15-20, 15-21, 15-22, 15-23, 15-24, 15-25, 15-26, 15-27, 15-28, 15-29, 15-30, 16-17, 16-18, 16-19, 16-25, 16-21, 16-22, 16-23, 16-24, 16-25, 16-26, 16-27, 16-28, 16-29, 16-30, 17-18, 17-19, 17-20, 17-21, 17-22, 17-23, 17-24, 17-25, 17-26, 17-27, 17-28, 17-29, 17-30, 18-19, 18-20, 18-21, 18-22, 18-23, 18-24, 18-25, 18-26, 18-27, 18-28, 18-29, 18-30, 19-20, 19-21, 19-22, 19-23, 19-24, 19-25, 19-26, 19-29, 19-28, 19-29, 19-30, 20-21, 20-22, 20-23, 20-24, 20-25, 20-26, 20-27, 20-28, 20-29, 20-30, 21-22, 21-23, 21-24, 21-25, 21-26, 21-27, 21-28, 21-29, 21-30, 22-23, 22-24, 22-25, 22-26, 22-27, 22-28, 22-29, 22-30, 23-24, 23-25, 23-26, 23-27, 23-28, 23-29, 23-30, 24-25, 24-26, 24-27, 24-28, 24-29, 24-30, 25-26, 25-27, 25-28, 25-29, 25-30, 26-27, 26-28, 26-29, 26-30, 27-28, 27-29, 27-30, 28-29, 28-30, or 29-30 linked nucleotides.

As noted-above, REVERSIR compounds can be of any length. For example, in some embodiments, the REVERSIR compound is a modified oligonucleotide consisting of 6-30 nucleotides. For example, the REVERSIR compound can consist of 6, 7, 8, 9, 10, 11, 12, 13, 14, 15, 16, 17, 18, 19, 20, 21, 22, 23, 24, 25, 26, 27, 28, 29 or 30 linked nucleobases. In some embodiments, the REVERSIR compound consists of 6-17, 7-16 or 8-15 linked nucleobases.

In some embodiments, the REVERSIR compound is a modified oligonucleotide consisting of 8-15 (e.g., 8, 9, 10, 11, 12, 13, 14 or 15) linked nucleotides. In some embodiments, the REVERSIR compound is a modified oligonucleotide consisting of 6-12, 7-11 or 8-10 linked nucleobases. In some embodiments, the REVERSIR compound consists of 8-9 linked nucleobases.

As discussed herein, REVERSIR compounds are oligonucleotides, e.g., modified oligonucleotides, that are substantially complementary to at least one strand of a universal dsRNA agent. Now without wishing to be bound by a theory, REVERSIR compounds that are substantially complementary to the seed region of the antisense strand of the dsRNA (i.e., at positions 2-8 of the 5′-end of the antisense strand) are particularly effective in reducing siRNA activity. Thus, in many embodiments, the REVERSIR compound is substantially complementary to nucleotides 2-8, 2-9, 2-10, 2-11, 2-12, 2-13, 2-14, 2-15 or 2-16 of the antisense strand of the universal dsRNA agents described herein. By substantially complementary in this context is meant a complementarity of at least 90%, preferably at least 95%, and more preferably complete complementarity.

### A. Modified REVERSIR Compounds Comprising Motifs of the Invention

The present invention also includes oligomeric compounds which are chimeric oligomeric compounds, i.e. chimeric REVERSIR compounds. “Chimeric” oligomeric compounds or “chimeras,” in the context of this invention, are oligomeric compounds which contain two or more chemically distinct regions, each made up of at least one monomer unit, i.e., a modified or unmodified nucleotide in the case of an oligonucleotide. Chimeric oligomeric compounds can be described as having a particular motif. In some embodiments, the motifs include, but are not limited to, an alternating motif, a gapped motif, a hemimer motif, a uniformly fully modified motif and a positionally modified motif. As used herein, the phrase “chemically distinct region” refers to an oligomeric region which is different from other regions by having a modification that is not present elsewhere in the oligomeric compound or by not having a modification that is present elsewhere in the oligomeric compound. An oligomeric compound can comprise two or more chemically distinct regions. As used herein, a region that comprises no modifications is also considered chemically distinct.

A chemically distinct region can be repeated within an oligomeric compound. Thus, a pattern of chemically distinct regions in an oligomeric compound can be realized such that a first chemically distinct region is followed by one or more second chemically distinct regions. This sequence of chemically distinct regions can be repeated one or more times. Preferably, the sequence is repeated more than one time. Both strands of a double-stranded oligomeric compound can comprise these sequences. Each chemically distinct region can actually comprise as little as a single monomers, e.g., nucleotides. In some embodiments, each chemically distinct region comprises 1, 2, 3, 4, 5, 6, 7, 8, 9, 10, 11, 12, 13, 14, 15, 16, 17 or 18 monomers, e.g., nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in an oligomeric compound have the same modification and all the even numbered nucleotides have a modification that is not present in the odd number nucleotides and vice versa.

In some embodiments, the oligonucleotide comprises two chemically distinct regions, wherein each region is 1, 2, 3, 4, 5, 6, 7, 8, 9 or 10 nucleotides in length.

In other embodiments, the oligomeric compound comprises three chemically distinct region. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to an oligomeric compound comprising a contiguous sequence of linked monomer subunits wherein the monomer subunits have two different types of sugar groups that alternate for essentially the entire sequence of the oligomeric compound. Oligomeric compounds having an alternating motif can be described by the formula: 5′-A(-L-B-L-A)n(-L-B)nn-3′ where A and B are monomelic subunits that have different sugar groups, each L is an internucleoside linking group, n is from about 4 to about 12 and nn is 0 or 1. This permits alternating oligomeric compounds from about 9 to about 26 monomer subunits in length. This length range is not meant to be limiting as longer and shorter oligomeric compounds are also amenable to the present invention. In one embodiment, one of A and B is a 2′-modified nucleoside as provided herein.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “type region” refers to a portion of an oligomeric compound wherein the nucleosides and internucleoside linkages within the region all comprise the same type of modifications; and the nucleosides and/or the internucleoside linkages of any neighboring portions include at least one different type of modification. As used herein the term “uniformly fully modified motif” refers to an oligonucleotide comprising a contiguous sequence of linked monomer subunits that each have the same type of sugar group. In one embodiment, the uniformly fully modified motif includes a contiguous sequence of nucleosides of the invention. In one embodiment, one or both of the 3′ and 5′-ends of the contiguous sequence of the nucleosides provided herein, comprise terminal groups such as one or more unmodified nucleosides.

As used herein the term “hemimer motif” refers to an oligomeric compound having a short contiguous sequence of monomer subunits having one type of sugar group located at the 5′ or the 3′ end wherein the remainder of the monomer subunits have a different type of sugar group. In general, a hemimer is an oligomeric compound of uniform sugar groups further comprising a short region (1, 2, 3, 4 or about 5 monomelic subunits) having uniform but different sugar groups and located on either the 3′ or the 5′ end of the oligomeric compound. In one embodiment, the hemimer motif comprises a contiguous sequence of from about 10 to about 28 monomer subunits of one type with from 1 to 5 or from 2 to about 5 monomer subunits of a second type located at one of the termini. In one embodiment, a hemimer is a contiguous sequence of from about 8 to about 20 β-D-2′-deoxyribonucleosides having from 1-12 contiguous nucleosides of the invention located at one of the termini. In one embodiment, a hemimer is a contiguous sequence of from about 8 to about 20 β-D-2′-deoxyribonucleosides having from 1-5 contiguous nucleosides of the invention located at one of the termini. In one embodiment, a hemimer is a contiguous sequence of from about 12 to about 18 β-D-2′-deoxyribo-nucleosides having from 1-3 contiguous nucleosides of the invention located at one of the termini. In one embodiment, a hemimer is a contiguous sequence of from about 10 to about 14 β-D-2′-deoxyribonucleosides having from 1-3 contiguous nucleosides of the invention located at one of the termini.

As used herein the term “blockmer motif” refers to an oligonucleotide comprising an otherwise contiguous sequence of monomer subunits wherein the sugar groups of each monomer subunit is the same except for an interrupting internal block of contiguous monomer subunits having a different type of sugar group. A blockmer overlaps somewhat with a gapmer in the definition but typically only the monomer subunits in the block have non-naturally occurring sugar groups in a blockmer and only the monomer subunits in the external regions have non-naturally occurring sugar groups in a gapmer with the remainder of monomer subunits in the blockmer or gapmer being β-D-2′-deoxyribonucleosides or β-D-ribonucleosides. In one embodiment, blockmer oligonucleotides are provided herein wherein all of the monomer subunits comprise non-naturally occurring sugar groups.

As used herein the term “positionally modified motif” is meant to include an otherwise contiguous sequence of monomer subunits having one type of sugar group that is interrupted with two or more regions of from 1 to about 5 contiguous monomer subunits having another type of sugar group. Each of the two or more regions of from 1 to about 5 contiguous monomer subunits are independently uniformly modified with respect to the type of sugar group. In one embodiment, each of the two or more regions have the same type of sugar group. In one embodiment, each of the two or more regions have a different type of sugar group. In one embodiment, positionally modified oligonucleotides are provided comprising a sequence of from 8 to 20 β-D-2′-deoxyribonucleosides that further includes two or three regions of from 2 to about 5 contiguous nucleosides of the invention. Positionally modified oligonucleotides are distinguished from gapped motifs, hemimer motifs, blockmer motifs and alternating motifs because the pattern of regional substitution defined by any positional motif does not fit into the definition provided herein for one of these other motifs. The term positionally modified oligomeric compound includes many different specific substitution patterns.

As used herein the term “gapmer” or “gapped oligomeric compound” refers to an oligomeric compound having two external regions or wings and an internal region or gap. The three regions form a contiguous sequence of monomer subunits with the sugar groups of the external regions being different than the sugar groups of the internal region and wherein the sugar group of each monomer subunit within a particular region is the same. When the sugar groups of the external regions are the same the gapmer is a symmetric gapmer and when the sugar group used in the 5′-external region is different from the sugar group used in the 3′-external region, the gapmer is an asymmetric gapmer. In one embodiment, the external regions are small (each independently 1, 2, 3, 4 or about 5 monomer subunits) and the monomer subunits comprise non-naturally occurring sugar groups with the internal region comprising β-D-2′-deoxyribonucleosides. In one embodiment, the external regions each, independently, comprise from 1 to about 5 monomer subunits having non-naturally occurring sugar groups and the internal region comprises from 6 to 18 unmodified nucleosides. The internal region or the gap generally comprises β-D-2′-deoxyribo-nucleosides but can comprise non-naturally occurring sugar groups.

In one embodiment, the gapped oligomeric compounds comprise an internal region of β-D-2′-deoxyribonucleosides with one of the external regions comprising nucleosides of the invention. In one embodiment, the gapped oligonucleotide comprise an internal region of β-D-2′-deoxyribonucleosides with both of the external regions comprising nucleosides of the invention. In one embodiment, the gapped oligonucleotide comprise an internal region of β-D-2′-deoxyribonucleosides with both of the external regions comprising nucleosides of the invention. In one embodiment, gapped oligonucleotides are provided herein wherein all of the monomer subunits comprise non-naturally occurring sugar groups. In one embodiment, gapped oliogonucleotides are provided comprising one or two nucleosides of the invention at the 5′-end, two or three nucleosides of the invention at the 3′-end and an internal region of from 10 to 16 β-D-2′-deoxyribonucleosides. In one embodiment, gapped oligonucleotides are provided comprising one nucleoside of the invention at the 5′-end, two nucleosides of the invention at the 3′-end and an internal region of from 10 to 16 β-D-2′-deoxyribonucleosides. In one embodiment, gapped oligonucleotides are provided comprising two nucleosides of the invention at the 5′-end, two nucleosides of the invention at the 3′-end and an internal region of from 10 to 14 β-D-2′-deoxyribonucleosides. In one embodiment, gapped oligonucleotides are provided that are from about 10 to about 21 monomer subunits in length. In one embodiment, gapped oligonucleotides are provided that are from about 12 to about 16 monomer subunits in length. In one embodiment, gapped oligonucleotides are provided that are from about 12 to about 14 monomer subunits in length.

In certain embodiments, the 5′-terminal monomer of an oligomeric compound of the invention comprises a phosphorous moiety at the 5′-end. In certain embodiments the 5′-terminal monomer comprises a 2′-modification. In certain such embodiments, the 2′-modification of the 5′-terminal monomer is a cationic modification. In certain embodiments, the 5′-terminal monomer comprises a 5′-modification. In certain embodiments, the 5′-terminal monomer comprises a 2′-modification and a 5′-modification. In certain embodiments, the 5′-terminal monomer is a 5′-stabilizing nucleoside. In certain embodiments, the modifications of the 5′-terminal monomer stabilize the 5′-phosphate. In certain embodiments, oligomeric compounds comprising modifications of the 5′-terminal monomer are resistant to exonucleases. In certain embodiments, oligomeric compounds comprising modifications of the 5′-terminal monomer have improved REVERSIR properties. In certain such embodiments, oligomeric compound comprising modifications of the 5′-terminal monomer have improved association with a strand of the siRNA.

In certain embodiments, the 5′-terminal monomer is attached to rest of the oligomeric compound a modified linkage. In certain such embodiments, the 5′-terminal monomer is attached to rest of the oligomeric compound by a phosphorothioate linkage.

In certain embodiments, oligomeric compounds of the present invention comprise one or more regions of alternating modifications. In certain embodiments, oligomeric compounds comprise one or more regions of alternating nucleoside modifications. In certain embodiments, oligomeric compounds comprise one or more regions of alternating linkage modifications. In certain embodiments, oligomeric compounds comprise one or more regions of alternating nucleoside and linkage modifications.

In certain embodiments, oligomeric compounds of the present invention comprise one or more regions of alternating 2′-F modified nucleosides and 2′-OMe modified nucleosides. In certain such embodiments, such regions of alternating 2′F modified and 2′OMe modified nucleosides also comprise alternating linkages. In certain such embodiments, the linkages at the 3′ end of the 2′-F modified nucleosides are phosphorothioate linkages. In certain such embodiments, the linkages at the 3′ end of the 2′OMe nucleosides are phosphodiester linkages.

In certain embodiments, such alternating regions are:

(2′-F)—(PS)-(2′-OMe)-(PO)

In certain embodiments, oligomeric compounds comprise 2, 3, 4, 5, 6, 7, 8, 9, 10, or 11 such alternating regions. Such regions may be contiguous or may be interrupted by differently modified nucleosides or linkages.

In certain embodiments, one or more alternating regions in an alternating motif include more than a single nucleoside of a type. For example, oligomeric compounds of the present invention may include one or more regions of any of the following nucleoside motifs:
ABA;
ABBA;
AABA;
AABBAA;
ABBABB;
AABAAB;
ABBABAABB;
ABABAA;
AABABAB;
ABABAA;
ABBAABBABABAA;
BABBAABBABABAA; or
ABABBAABBABABAA;
wherein A is a nucleoside of a first type and B is a nucleoside of a second type. In certain embodiments, A and B are each selected from 2′-F, 2′-OMe, LNA, DNA and MOE.

In certain embodiments, A is DNA. In certain embodiments B is DNA. In some embodiments, A is 4′-CH₂O-2′-LNA. In certain embodiments, B is 4′-CH₂O-2′-LNA. In certain embodiments, A is DNA and B is 4′-CH₂O-2′-LNA. In certain embodiments A is 4′-CH₂O-2′-LNA and B is DNA.

In certain embodiments, A is 2′-OMe. In certain embodiments B is 2′-OMe. In certain embodiments, A is 2′-OMe and B is 4′-CH₂O-2′-LNA. In certain embodiments A is 4′-CH₂O-2′-LNA and B is 2′-OMe. In certain embodiments, A is 2′-OMe and B is DNA. In certain embodiments A is DNA and B is 2′-OMe.

In certain embodiments, A is (S)-cEt. In some embodiments, B is (S)-cEt. In certain embodiments, A is 2′-OMe and B is (S)-cEt. In certain embodiments A is (S)-cEt and B is 2′-OMe.

In certain embodiments, A is DNA and B is (S)-cEt. In certain embodiments A is (S)-cEt and B is DNA.

In certain embodiments, A is 2′-F. In certain embodiments B is 2′-F. In certain embodiments, A is 2′-F and B is 4′-CH₂O-2′-LNA. In certain embodiments A is 4′-CH₂O-2′-LNA and B is 2′-F. In certain embodiments, A is 2′-F and B is (S)-cEt. In certain embodiments A is (S)-cEt and B is 2′-F . . . . In certain embodiments, A is 2′-F and B is DNA. In certain embodiments A is DNA and B is 2′-F. In certain embodiments, A is 2′-OMe and B is 2′-F. In certain embodiments, A is DNA and B is 2′-OMe. In certain embodiments, A is 2′-OMe and B is DNA.

In certain embodiments, oligomeric compounds having such an alternating motif also comprise a 5′ terminal nucleoside comprising a phosphate stabilizing modification. In certain embodiments, oligomeric compounds having such an alternating motif also comprise a 5′ terminal nucleoside comprising a 2′-cationic modification. In certain embodiments, oligomeric compounds having such an alternating motif also comprise a 5′ terminal modification.

In certain embodiments, oligomeric compounds of the present invention comprise a region having a 2-2-3 motif. Such regions comprises the following motif:

5′-(E)_{w}-(A)₂-(B)ₓ-(A)₂-(C)_{y}-(A)₃-(D)_{z}
wherein: A is a first type of modified nucleoside;
B, C, D, and E are nucleosides that are differently modified than A, however, B, C, D, and E may have the same or different modifications as one another;
w and z are from 0 to 15;
x and y are from 1 to 15.

In certain embodiments, A is a 2′-OMe modified nucleoside. In certain embodiments, B, C, D, and E are all 2′-F modified nucleosides. In certain embodiments, A is a 2′-OMe modified nucleoside and B, C, D, and E are all 2′-F modified nucleosides.

In certain embodiments, the linkages of a 2-2-3 motif are all modified linkages. In certain embodiments, the linkages are all phosphorothioate linkages. In certain embodiments, the linkages at the 3′-end of each modification of the first type are phosphodiester.

In certain embodiments, Z is 0. In such embodiments, the region of three nucleosides of the first type are at the 3′-end of the oligonucleotide. In certain embodiments, such region is at the 3′-end of the oligomeric compound, with no additional groups attached to the 3′ end of the region of three nucleosides of the first type. In certain embodiments, an oligomeric compound comprising an oligonucleotide where Z is 0, may comprise a terminal group attached to the 3′-terminal nucleoside. Such terminal groups may include additional nucleosides. Such additional nucleosides are typically non-hybridizing nucleosides.

In certain embodiments, Z is 1-3. In certain embodiments, Z is 2. In certain embodiments, the nucleosides of Z are 2′-MOE nucleosides. In certain embodiments, Z represents non-hybridizing nucleosides. To avoid confusion, it is noted that such non-hybridizing nucleosides might also be described as a 3′-terminal group with Z=0.

It is to be understood, that certain of the above described motifs and modifications can be combined. Since a motif may comprise only a few nucleosides, a particular oligomeric compound can comprise two or more motifs. By way of non-limiting example, in certain embodiments, oligomeric compounds can have two or more nucleotide motifs selected from LNAs, phosphorthioate linkages, 2′-OMe, conjugated ligand(s).

Oligomeric compounds having any of the various nucleoside motifs described herein, can have also have any linkage motif. For example, in the oligomeric compounds first 1, 2, 3, 4 or 5 at the 5′-end be modified intrersugar linkages and first 4, 5, 6, 7 or 8 intersugar linkages at the 3′-end can be modified intersugar linkages. The central region of such modified oligomeric compound can have intersugar linkages based on the any of the other motifs described herein, for example, uniform, alternating, hemimer, gapmer, and the like. In some embodiments, the oligomeric compound comprise a phosphorothioate linkage between the first and second monomer at the 5′-terminus, alternating phosphorothioate/phosphodiester linkages in the central region and 6, 7, or 8 phosphorothioate linkages at the 3′-terminus.

It is to be noted that the lengths of the regions defined by a nucleoside motif and that of a linkage motif need not be the same.

In some embodiments, single-stranded oligomeric compounds include at least one of the following motifs:
(a) 5′-phosphorothioate or 5′-phosphorodithioate;
(b) a cationic modification of nucleotides 1 and 2 on the 5′ terminal, wherein the cationic modification is at C5 position of pyrimidines and C2, C6, C8, exocyclic N2 or exocyclic N6 of purines;
(c) at least one G-clamp nucleotide in the first two terminal nucleotides at the 5′ end and the other nucleotide having a cationic modification, wherein the cationic modification is at C5 position of pyrimidines or C2, C6, C8, exocyclic N2 or exocyclic N6 position of purines;
(d) at least one 2′-F modified nucleotide comprising a nucleobase base modification;
(e) at least one gem-2′-O-methyl/2′-F modified nucleotide comprising a nucleobase modification, preferably the methyl substituent is in the up configuration, e.g. in the arabinose configuration;
(f) a 5′-PuPu-3′ dinucleotide at the 3′ terminal wherein both nucleotides comprise a modified MOE at 2′-position as described in U.S. Patent Application Publication No. 20130130378, content of which is incorporated herein by reference in its entirety,
(g) a 5′-PuPu-3′ dinucleotide at the 5′ terminal wherein both nucleotides comprise a modified MOE at 2′-position as described in U.S. Patent Application Publication No. 20130130378;
(h) nucleotide at the 5′ terminal having a modified MOE at 2′-position as described in U.S. Patent Application Publication No. 20130130378;
(i) nucleotide at the 5′ terminal having a 3′-F modification;
(j) 5′ terminal nucleotide comprising a 4′-substituent;
(k) 5′ terminal nucleotide comprising a 04′ modification;
(l) 3′ terminal nucleotide comprising a 4′-substituent; and
(m) combinations thereof.

The above examples are provided solely to illustrate how the described motifs may be used in combination and are not intended to limit the invention to the particular combinations or the particular modifications used in illustrating the combinations. Further, specific examples herein are intended to encompass more generic embodiments. All of the examples throughout this specification contemplate such generic interpretation.

It is also noted that the lengths of oligomeric compounds can be easily manipulated by lengthening or shortening one or more of the described regions, without disrupting the motif.

In some embodiments, oligomeric compound comprises two or more chemically distinct regions and has a structure as described in International Application No. PCT/US09/038433, filed Mar. 26, 2009, contents of which are herein incorporated in their entirety.

### V. Universal dsRNA Agents and REVERSIR Compounds of the Invention Comprising Ligands

Another modification of the RNA of a universal iRNA of the invention or of a monomer of a REVERSIR compound of the invention involves chemically linking to the iRNA or REVERSIR compound one or more ligands, moieties or conjugates that enhance the activity, cellular distribution, or cellular uptake of the iRNA or REVERSIR compound e.g., into a cell. Such moieties include but are not limited to lipid moieties such as a cholesterol moiety (Letsinger et al., Proc. Natl. Acid. Sci. USA, 1989, 86:6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting, or lifetime of an iRNA agent or REVERSIR compound into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. In some embodiments, ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent or REVERSIR compound into the cell, for example, by disrupting the cell's cytoskeleton, e.g., by disrupting the cell's microtubules, microfilaments, or intermediate filaments. The drug can be, for example, taxol, vincristine, vinblastine, cytochalasin, nocodazole, japlakinolide, latrunculin A, phalloidin, swinholide A, indanocine, or myoservin.

In some embodiments, a ligand attached to an iRNA or REVERSIR compound as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs and REVERSIR compounds of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other methods for such synthesis known in the art may additionally or alternatively be employed. It is also known to use similar techniques to prepare other oligonucleotides, such as the phosphorothioates and alkylated derivatives.

In the ligand-conjugated iRNAs, ligand-conjugated REVERSIR compounds, and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. In one embodiment, such a lipid or lipid-based molecule binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid based ligand binds HSA. In one embodiment, it binds HSA with a sufficient affinity such that the conjugate will be distributed to a non-kidney tissue. However, it is preferred that the affinity not be so strong that the HSA-ligand binding cannot be reversed.

In other embodiments, the lipid based ligand binds HSA weakly or not at all. In one embodiment, the conjugate will be distributed to the kidney. Other moieties that target to kidney cells can also be used in place of, or in addition to, the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells.

Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

### B. Cell Permeation Agents

In another aspect, the ligand is a cell-permeation agent, such as, a helical cell-permeation agent. In one embodiment, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In one embodiment, the helical agent is an alpha-helical agent, which has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents or REVERSIR compounds can affect pharmacokinetic distribution of the iRNA or REVERSIR compound, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 1). An RFGF analogue (e.g., amino acid sequence AALLPVLLAAP (SEQ ID NO:2) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:3) and the Drosophila Antennapedia protein (RQIKIWFQNRRMKWKK (SEQ ID NO:4) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand, e.g., PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, a universal iRNA or REVERSIR compound further comprises a carbohydrate. The carbohydrate conjugated iRNA and carbohydrate-conjugated REVERSIR compound are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri-, and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In certain embodiments, a carbohydrate conjugate for use in the compositions and methods of the invention is a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference. In some embodiments, the GalNAc conjugate serves as a ligand that targets the iRNA or REVERSIR compound to particular cells. In some embodiments, the GalNAc conjugate targets the iRNA or REVERSIR compound to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand of the dsRNA or to the 3′ end of the REVERSIR compound. In some embodiments, the GalNAc conjugate is conjugated to the iRNA agent or REVERSIR compound (e.g., to the 3′ end of the sense strand) via a linker, e.g., a linker as described herein. In some embodiments the GalNAc conjugate is conjugated to the 5′ end of the sense strand of the dsRNA agent. In some embodiments the GalNAc conjugate is conjugated to the 5′ end of the sense strand of the REVERSIR compound. In some embodiments, the GalNAc conjugate is conjugated to the universal iRNA agent or REVERSIR compound (e.g., to the 5′ end of the sense strand) via a linker, e.g., a linker as described herein.

In certain embodiments of the invention, the GalNAc or GalNAc derivative is attached to a universal iRNA agent or REVERSIR compound of the invention via a monovalent linker. In some embodiments, the GalNAc or GalNAc derivative is attached to a universal iRNA agent or REVERSIR compound of the invention via a bivalent linker. In yet other embodiments of the invention, the GalNAc or GalNAc derivative is attached to a universal iRNA agent or REVERSIR compound of the invention via a trivalent linker. In other embodiments of the invention, the GalNAc or GalNAc derivative is attached to a universal iRNA agent or REVERSIR compound of the invention via a tetravalent linker.

In certain embodiments, the double stranded RNAi agents or REVERSIR compound of the invention comprise one GalNAc or GalNAc derivative attached to the iRNA agent or REVERSIR compound. In certain embodiments, the double stranded RNAi agents or REVERSIR compound of the invention comprise a plurality (e.g., 2, 3, 4, 5, or 6) GalNAc or GalNAc derivatives, each independently attached to a plurality of nucleotides of the double stranded RNAi agent or REVERSIR compound through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In one embodiment, a carbohydrate conjugate for use in the compositions and methods of the invention is selected from the group consisting of:

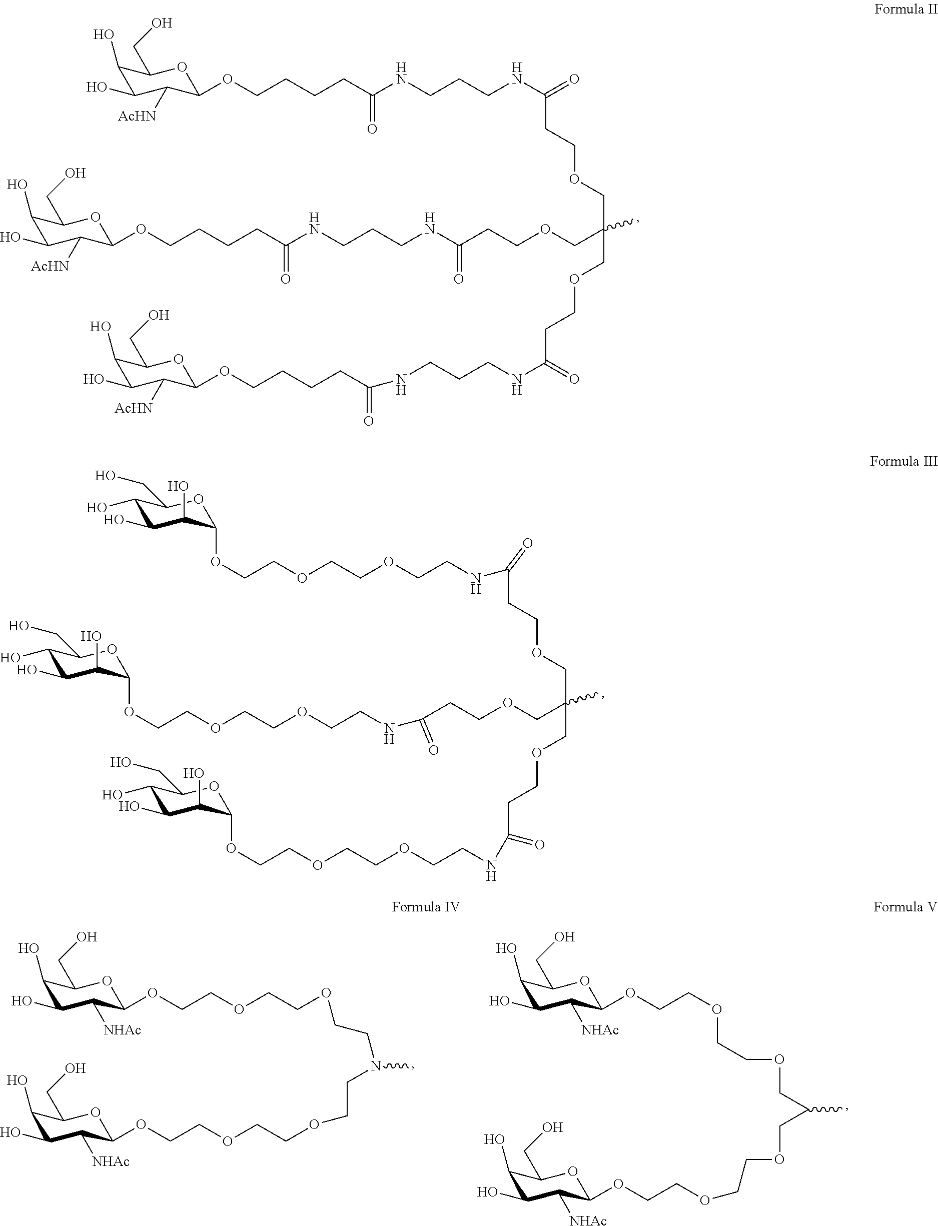

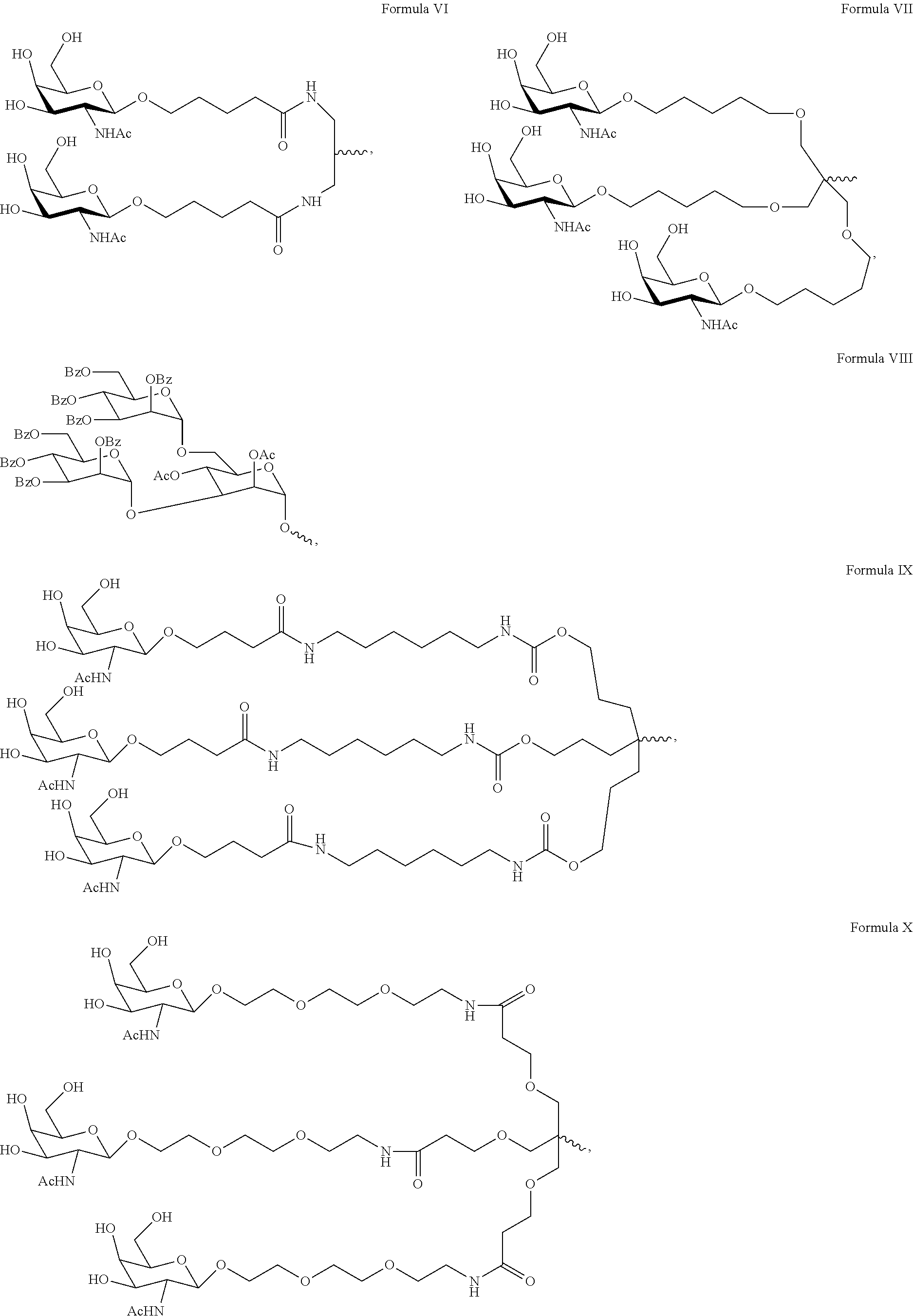

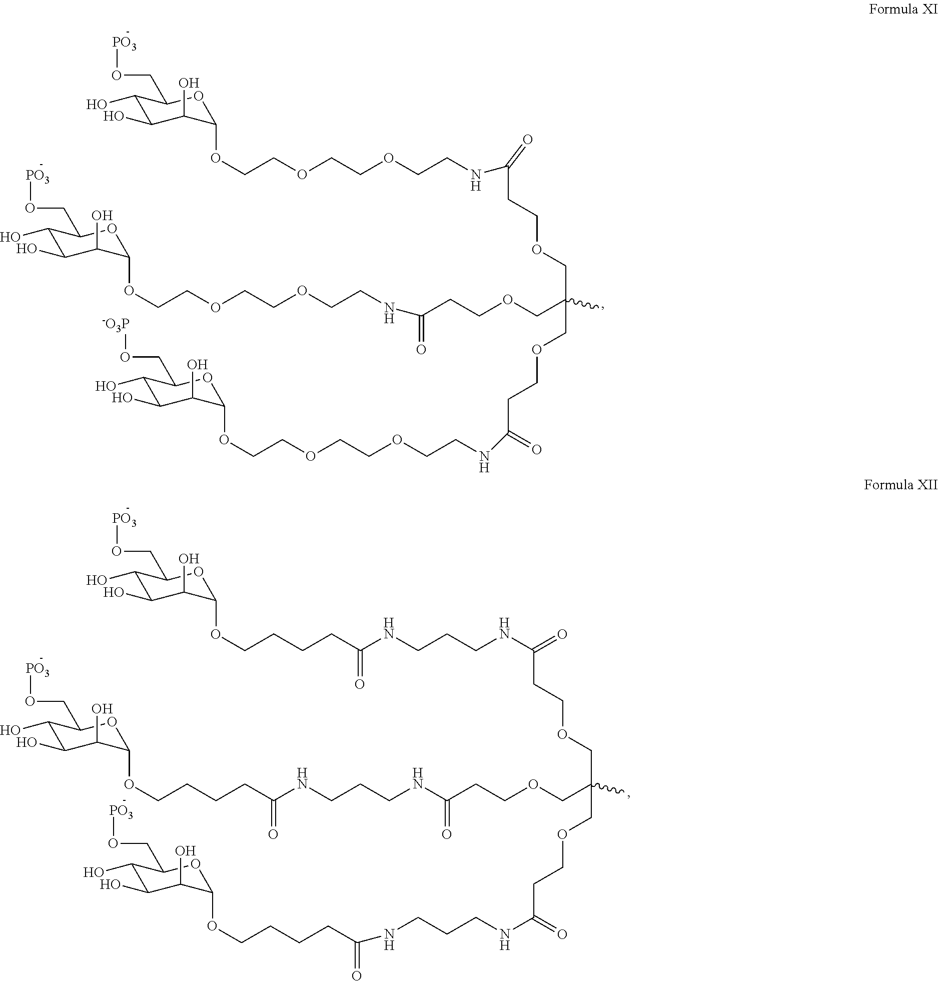

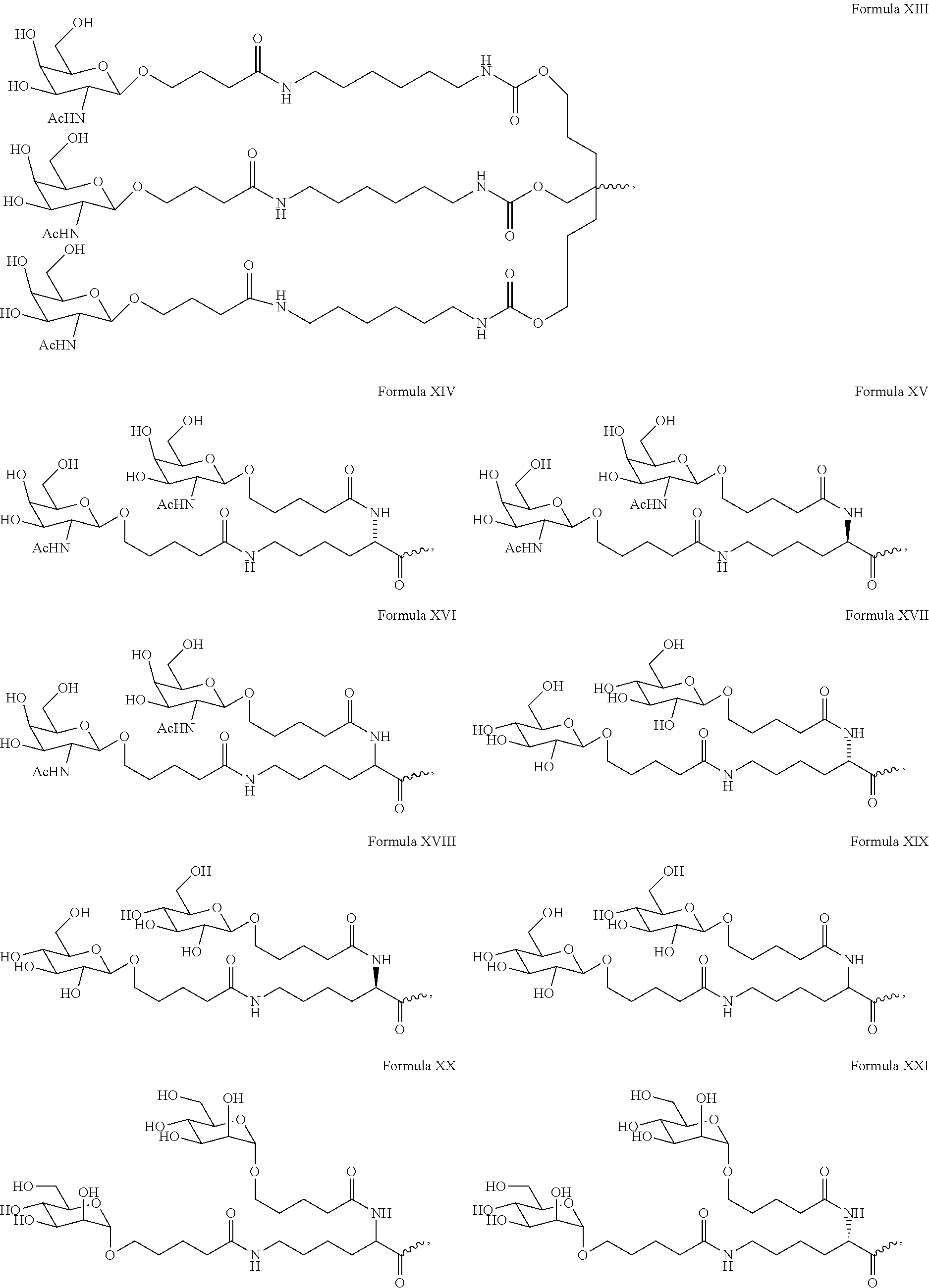

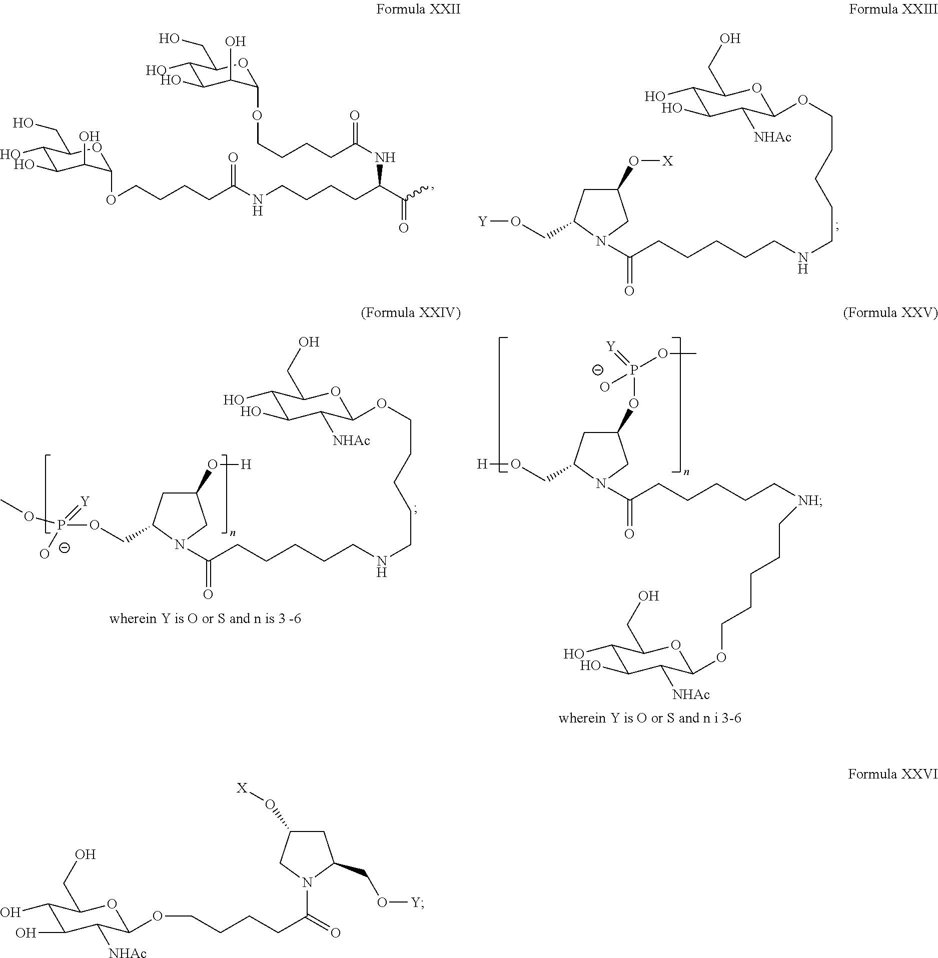

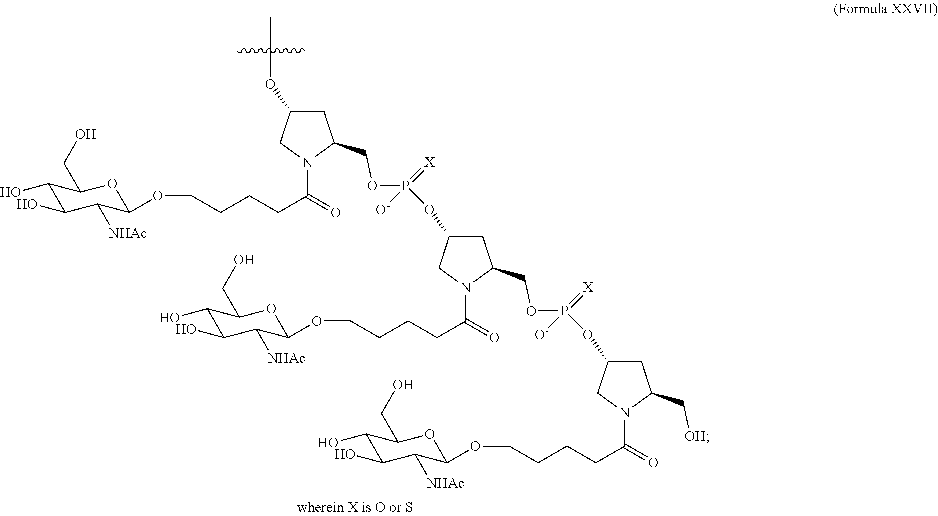

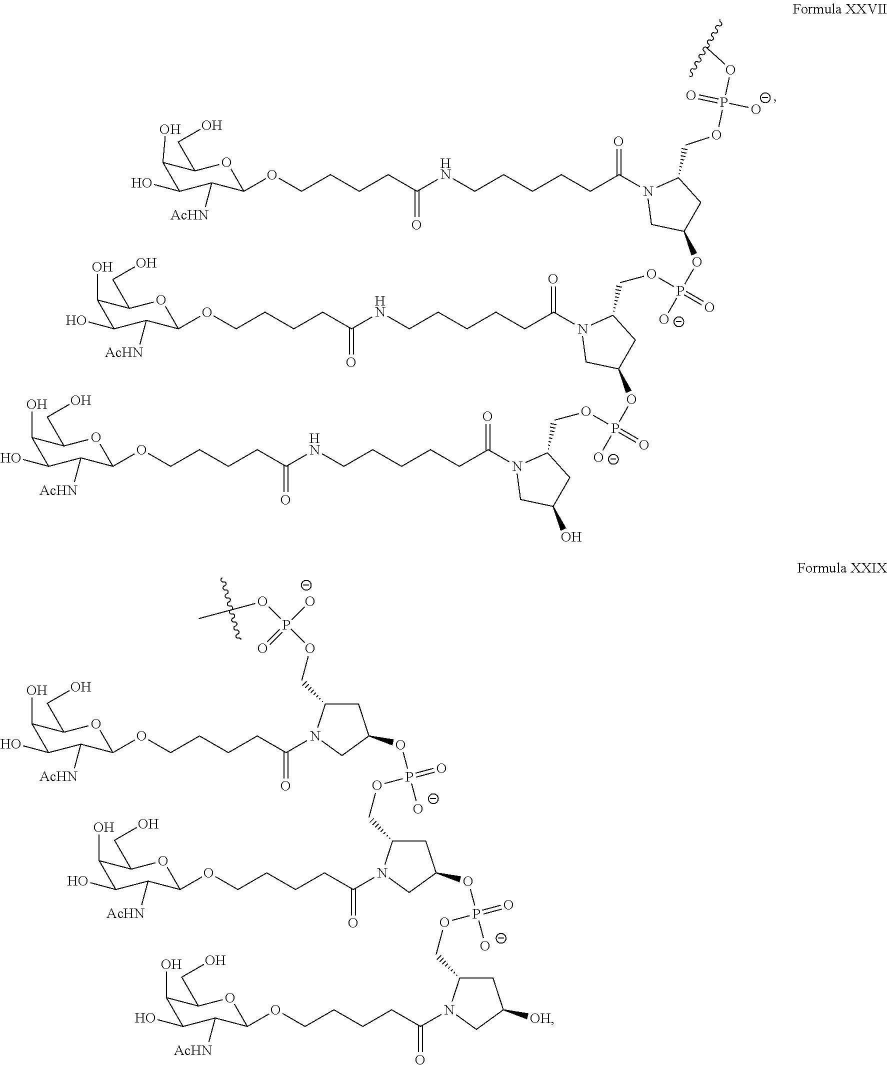 [SMILES: *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCCNC(=O)CCCCOC3OC(CO)C(O)C(O)C3NC(C)=O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCOC3OC(CO)C(O)C(O)C3NC(C)=O)CN2C(=O)CCCCOC2OC(CO)C(O)C(O)C2NC(C)=O)CN1C(=O)CCCCOC1OC(CO)C(O)C(O)C1NC(C)=O]

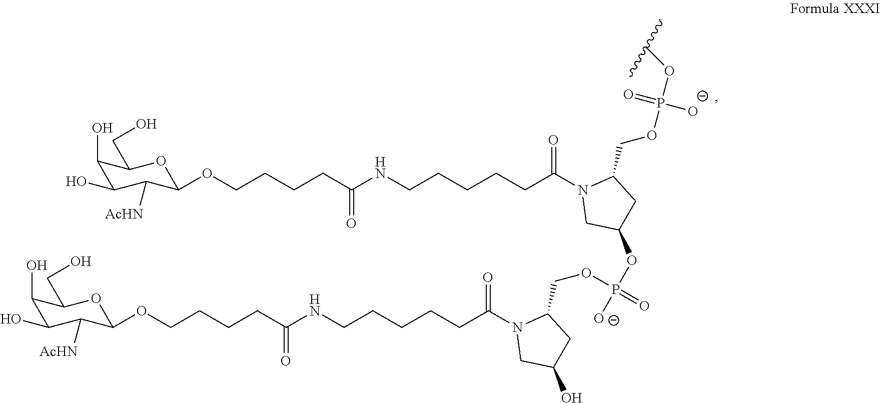 [SMILES: *OP(=O)([O-])OC[C@@H]1C[C@@H](OP(=O)([O-])OC[C@@H]2C[C@@H](O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O]

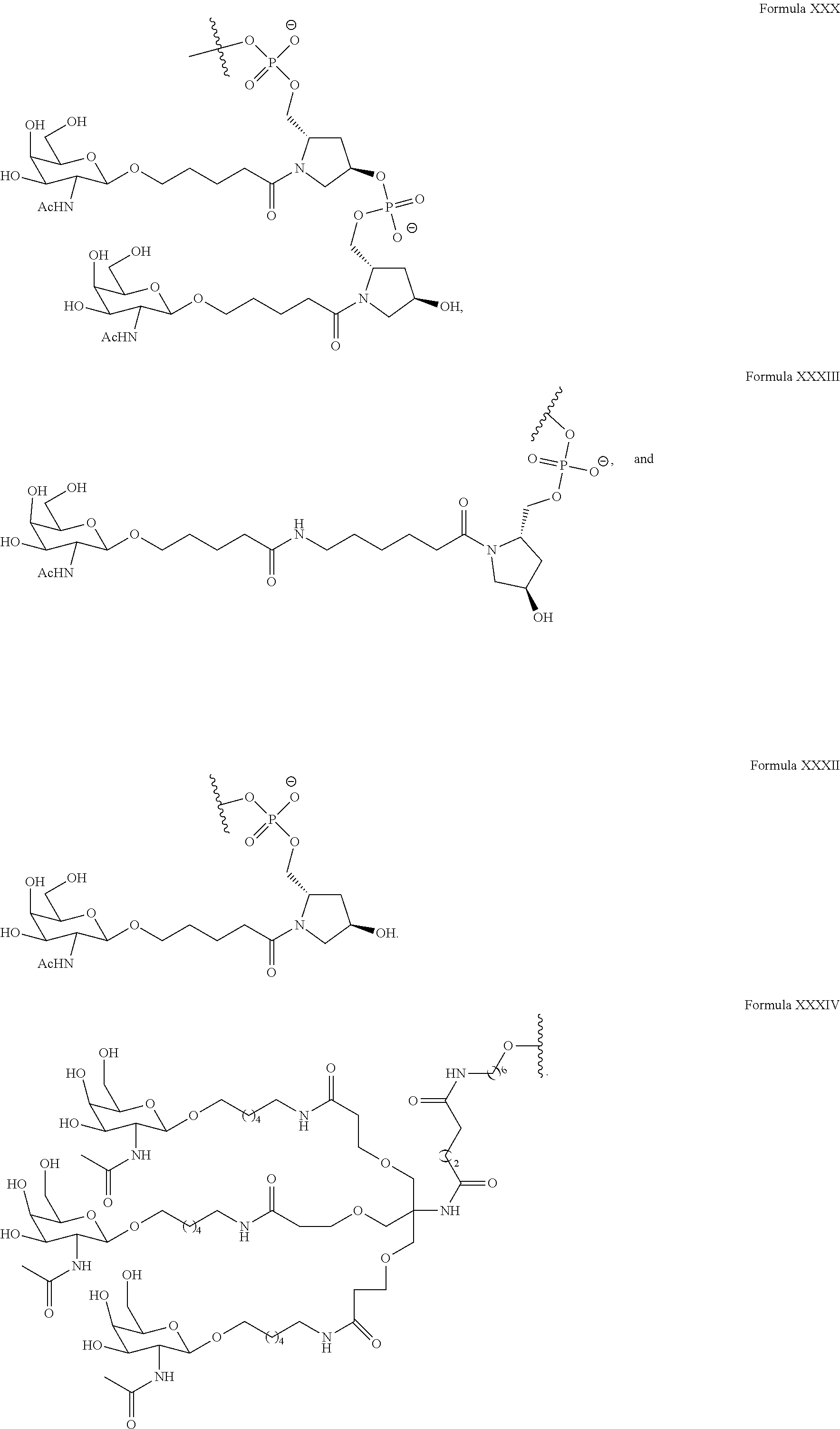 [SMILES: *OP(=O)([O-])OC[C@@H]1C[C@@H](OP(=O)([O-])OC[C@@H]2C[C@@H](O)CN2C(=O)CCCCOC2OC(CO)C(O)C(O)C2NC(C)=O)CN1C(=O)CCCCOC1OC(CO)C(O)C(O)C1NC(C)=O ; *OP(=O)([O-])OC[C@@H]1C[C@@H](O)CN1C(=O)CCCCCNC(=O)CCCCOC1OC(CO)C(O)C(O)C1NC(C)=O ; *OP(=O)([O-])OC[C@@H]1C[C@@H](O)CN1C(=O)CCCCOC1OC(CO)C(O)C(O)C1NC(C)=O ; *OCNC(=O)CCC(=O)NC(COCCC(=O)NCCCOC1OC(CO)C(O)C(O)C1NC(C)=O)(COCCC(=O)NCCCOC1OC(CO)C(O)C(O)C1NC(C)=O)COCCC(=O)NCCCOC1OC(CO)C(O)C(O)C1NC(C)=O]

In another embodiment, a carbohydrate conjugate for use in the compositions and methods of the invention is a monosaccharide. In one embodiment, the monosaccharide is an N-acetylgalactosamine, such as

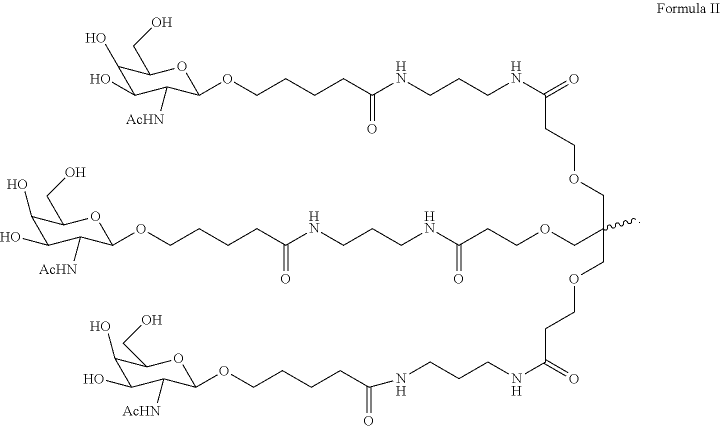 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent or REVERSIR compound is attached to the carbohydrate conjugate via a linker as shown in the following schematic, wherein X is O or S

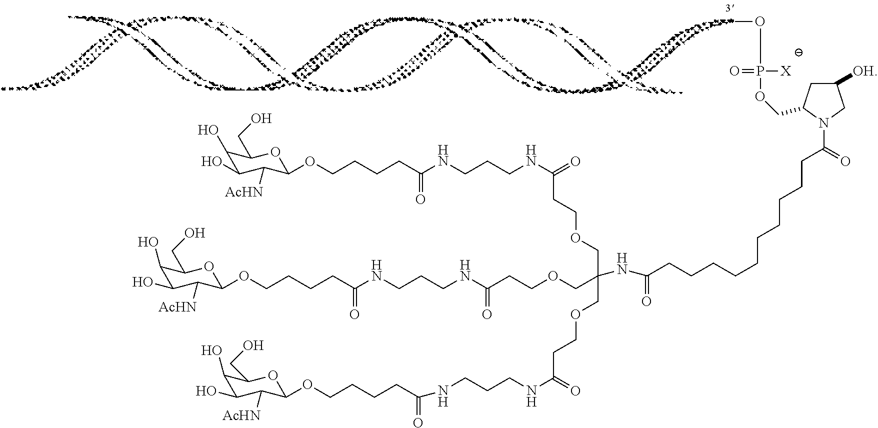

In some embodiments, the RNAi agent or REVERSIR compound is conjugated to L96 as defined in Table 1 and shown below:

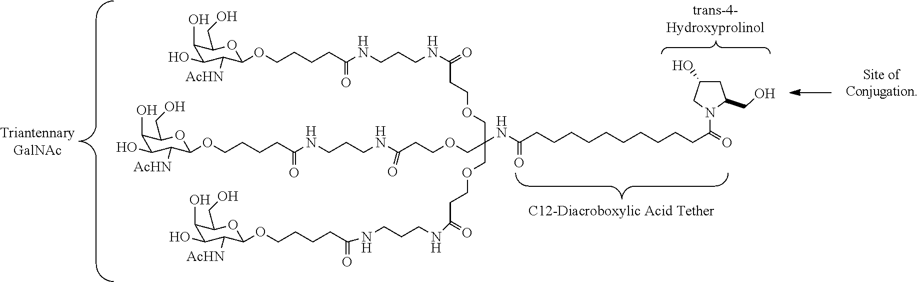

Another representative carbohydrate conjugate for use in the embodiments described herein includes, but is not limited to,

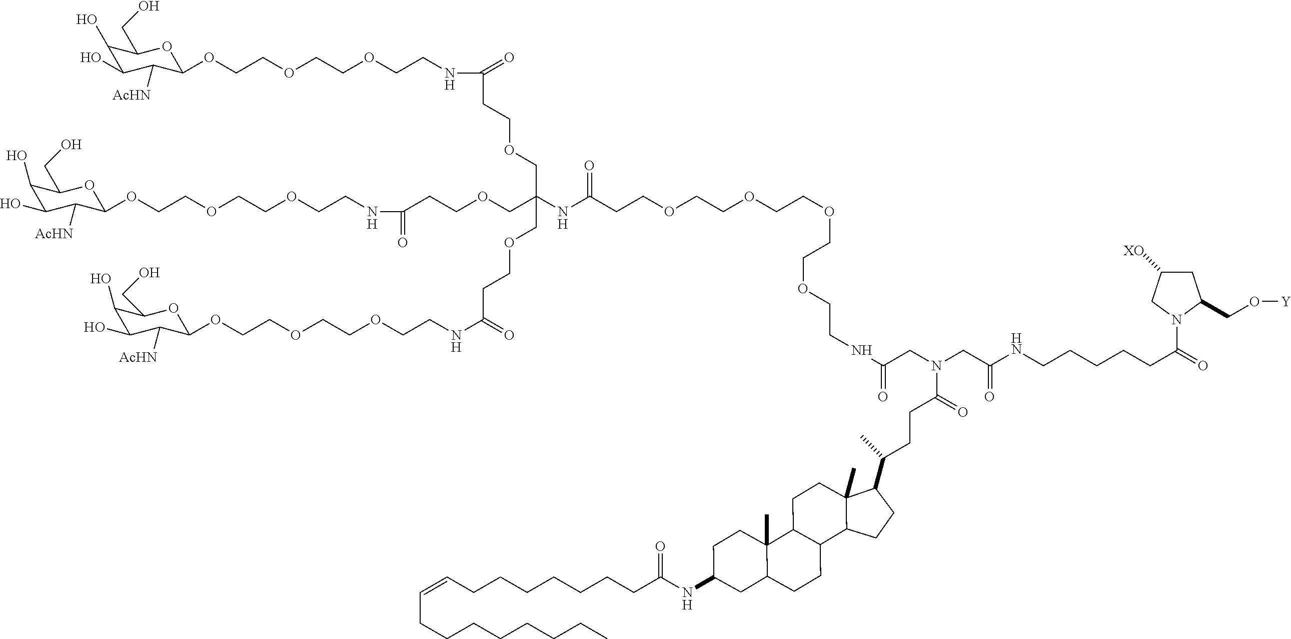

(Formula XXXVI), when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

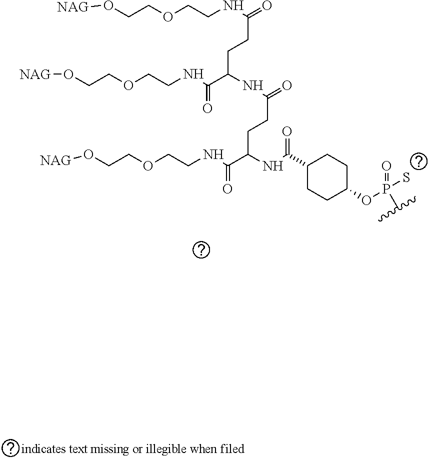

In certain embodiments of the invention, the GalNAc or GalNAc derivative is attached to an iRNA agent or REVERSIR compound of the invention via a monovalent linker. In some embodiments, the GalNAc or GalNAc derivative is attached to an iRNA agent or REVERSIR compound of the invention via a bivalent linker. In yet other embodiments of the invention, the GalNAc or GalNAc derivative is attached to an iRNA agent or REVERSIR compound of the invention via a trivalent linker.

In one embodiment, the double stranded RNAi agents or REVERSIR compound of the invention comprise one or more GalNAc or GalNAc derivative attached to the iRNA agent. The GalNAc may be attached to any nucleotide via a linker, e.g., on the sense strand or antsisense strand. The GalNac may be attached to the 5′-end of the sense strand, the 3′ end of the sense strand, the 5′-end of the antisense strand, or the 3′-end of the antisense strand, or in the case of the REVERSIR compound, to the 5′ or 3′ end of the oligonucleotide. In one embodiment, the GalNAc is attached to the 3′ end of the sense strand of a dsRNA, e.g., via a trivalent linker. In one embodiment, the GalNAc is attached to the 3′ end of the REVERSIR oligonucleotide, e.g., via a trivalend linker.

In other embodiments, the double stranded RNAi agents of the invention or REVERSIR compound comprise a plurality (e.g., 2, 3, 4, 5, or 6) GalNAc or GalNAc derivatives, each independently attached to a plurality of nucleotides of the double stranded RNAi agent or REVERSIR compound through a plurality of linkers, e.g., monovalent linkers.

In some embodiments, for example, when the two strands of an iRNA agent of the invention is part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in PCT Publication Nos. WO 2014/179620 and WO 2014/179627, the entire contents of each of which are incorporated herein by reference.

### D. Linkers

In some embodiments, the conjugate or ligand described herein can be attached to an iRNA oligonucleotide or REVERSIR compound with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

A cleavable linking group is one which is sufficiently stable outside the cell, but which upon entry into a target cell is cleaved to release the two parts the linker is holding together. In an exemplary embodiment, the cleavable linking group is cleaved at least about 10 times, 20, times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a selected pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety or REVERSIR compound and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In other embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Exemplary embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. In certain embodiments a phosphate-based linking group is —O—P(O)(OH)—O—. These candidates can be evaluated using methods analogous to those described above.

iii. Acid Cleavable Linking Groups

In other embodiments, a cleavable linker comprises an acid cleavable linking group. An acid cleavable linking group is a linking group that is cleaved under acidic conditions. In certain embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). An exemplary embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In other embodiments, a cleavable linker comprises an ester-based cleavable linking group. An ester-based cleavable linking group is cleaved by enzymes such as esterases and amidases in cells. Examples of ester-based cleavable linking groups include, but are not limited to, esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet other embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In some embodiments, an iRNA or REVERSIR compound of the invention is conjugated to a carbohydrate through a linker. Non-limiting examples of iRNA or REVERSIR compound carbohydrate conjugates with linkers of the compositions and methods of the invention include, but are not limited to,

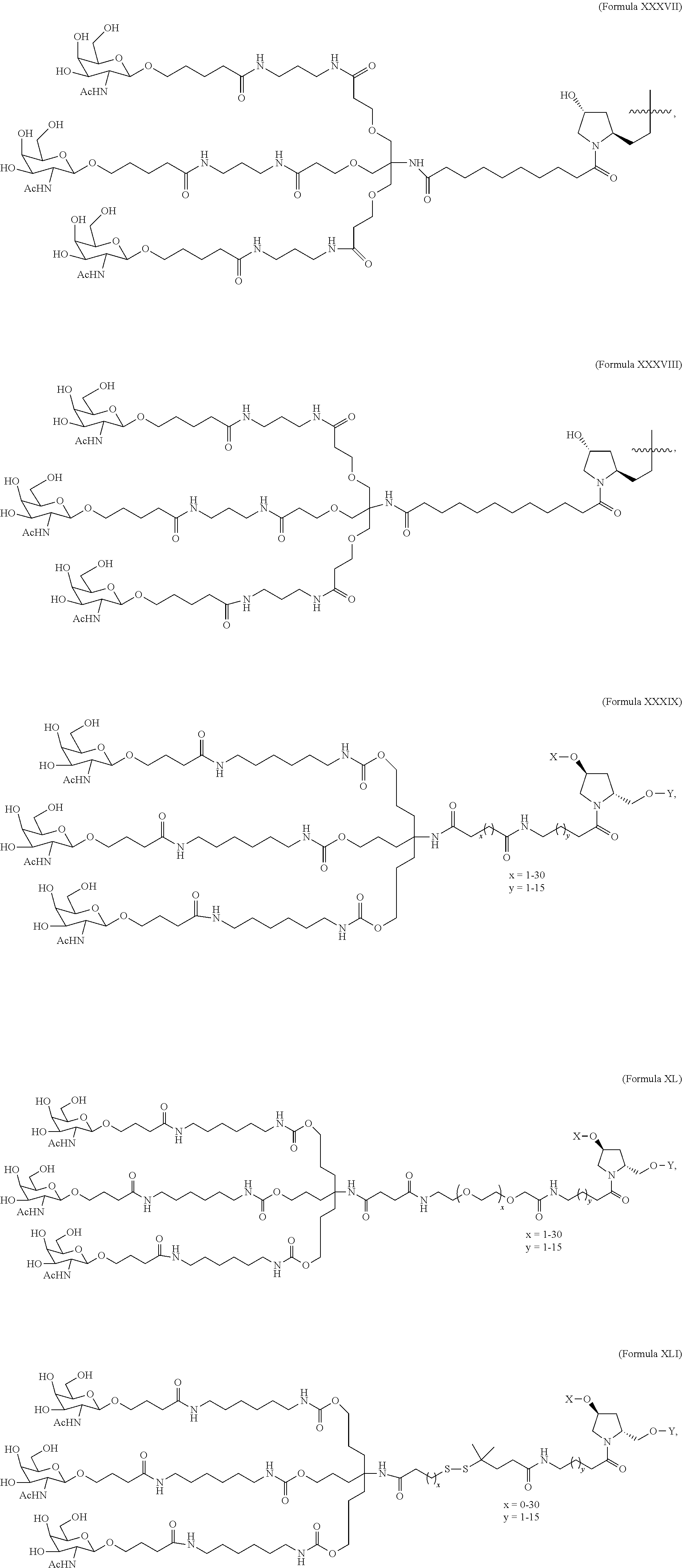

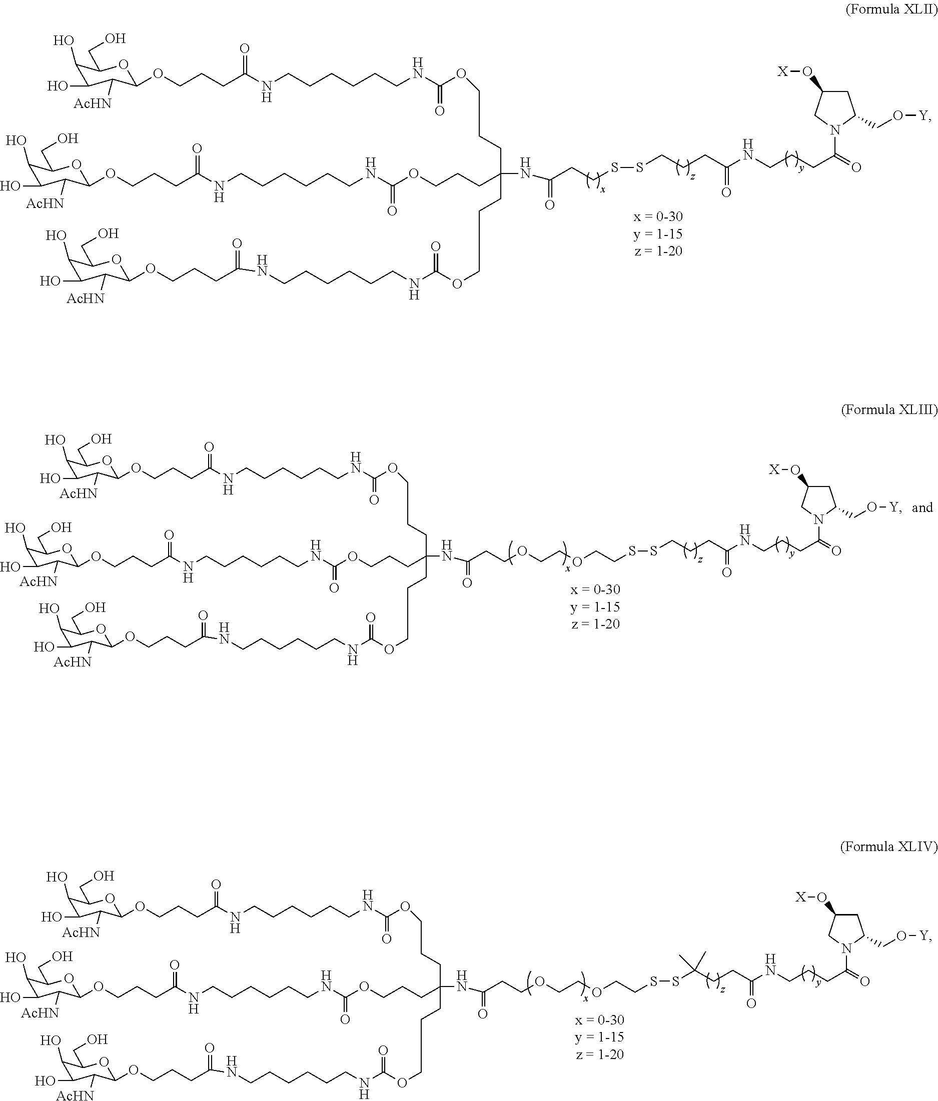

when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In one embodiment, a dsRNA or REVERSIR compound of the invention is conjugated to a bivalent or trivalent branched linker selected from the group of structures shown in any of formula (XLV)-(XLVI):

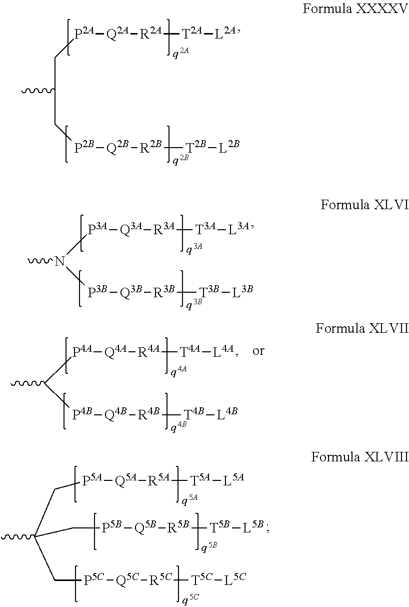

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

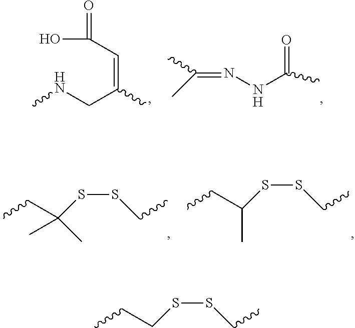

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a universal target site, such as those of formula (XLIX):

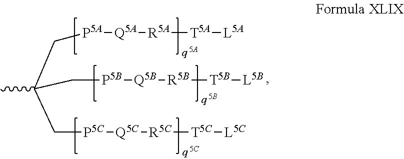

wherein L5A, L5B and L5C represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1): 54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### VI. Vectors and Systems of the Invention

The present inventing also provides systems comprising the universal dsRNA agents and/or REVERSIR compounds of the invention for use in various clinical settings or laboratory settings for on demand expression of a transgene, e.g., on-demand expression of a gene therapy transgene.

Accordingly, in one aspect, the present invention provides a system for on-demand expression of a transgene. The system includes an expression vector encoding a transgene and comprising a universal iRNA target site; a universal double stranded ribonucleic acid (dsRNA) agent, comprising a sense strand and an antisense strand forming a double stranded region, which recognizes and binds to the universal iRNA target site to thereby inhibit the expression of the transgene; and, optionally, a REVERSIR compound that abrogates the iRNA activity of the universal dsRNA agent to thereby allow the expression of the transgene.

In some embodiments, the expression vector comprises multiple, e.g., 2, 3, 4, 5, 6, 7, 8, 9, or 10, 11, 12, 13, 14, 15, 16, 17, 18, 19, or 20, or more, copies of the universal iRNA target site.

In another aspect, the present invention provides a system for on-demand expression of a transgene, which includes an expression vector encoding a transgene and a double stranded ribonucleic acid (dsRNA) agent targeting the transgene; wherein expression of the transgene is inhibited by the expression of the dsRNA agent targeting the transgene; and, optionally, a REVERSIR compound that abrogates the iRNA activity of the dsRNA agent to thereby allow the expression of the transgene.

In some embodiments, the expression vector comprises multiple, e.g., 2, 3, 4, 5, 6, 7, 8, 9, or 10, copies of the dsRNA agent targeting the transgene.

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes or nucleic acid molecules to which they are operatively linked and are referred to as “expression vectors” or “recombinant expression vectors.” Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

Expression vectors are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, adeno-associated viruses, lentiviruses), which serve equivalent functions.

The recombinant expression vectors of the invention comprise a universal target nucleotide sequence in a form suitable for expression of the universal target nucleotide sequence in a host cell, which means that the recombinant expression vectors include one or more regulatory sequences, selected on the basis of the host cells to be used for expression, which is operatively linked to the nucleotide sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). The expression vectors of the invention can be introduced into host cells to thereby produce proteins or portions thereof, including fusion proteins or portions thereof, encoded by nucleic acids as described herein.

In certain embodiments, the present invention provides a vector, such as an expression vector, e.g., a viral vector, e.g., an AAV vector, encoding a universal target sequence.

In certain embodiments, the expression vector further comprises a transgene.

A “transgene” is a gene that has been transferred by any of a number of genetic engineering techniques, from one organism to another.

Suitable transgenes for use in the present invention include, for example, any transgene encoding a gene useful for gene therapy to treat a genetic disease, e.g., any of the genetic diseases described on the website of the National Institutes of Health under the topic subsection Genetic Disorders (website at health.nih.gov/topic/GeneticDisorders), such as blood and coagulation diseases and disorders; bleeding disorders; cell dysregulation and oncology diseases and disorders; autoimmune diseases; metabolic, liver, kidney and protein diseases and disorders; muscular/skeletal diseases and disorders; neurological and neuronal diseases and disorders; occular diseases and disorders; epilepsy; Duchenne muscular dystrophy; schizophrenia; trinucleotide repeat disorders; Fragile X Syndrome; secretase related disorders; prion-related disorders; drug addiction, autism; Alzheimer's disease; Parkinson's disease; Down Syndrome; Cystic Fibrosis; Thalassemia; Sickle Cell Anemia; Huntington's Disease; and Tay-Sachs Disease.

Exemplary disorders that may be treated using the methods of the invention include any diseases and disorders described in US2019/0153471, the entire contents of which are incorporated herein by reference.

In one embodiment, the viral vector is a biscistronic vector. “Biscistronic vectors” permit the simultaneous expression of two proteins separately, but from the same RNA transcript.

The term “AAV vector” or “AAV construct” refers to a vector derived from an adeno-associated virus serotype, including without limitation, AAV-1, AAV-2, AAV-3, AAV-4, AAV-5, AAV6, AAV7, AAV8, and AAV9. “AAV vector” refers to a vector that includes AAV nucleotide sequences as well as heterologous nucleotide sequences. AAV vectors require only the 145 base terminal repeats in cis to generate virus. All other viral sequences are dispensable and may be supplied in trans (Muzyczka (1992) Curr. Topics Microbiol. Immunol. 158:97-129). In some embodiments, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging.

In particular embodiments, the AAV vector is an AAV8, AAV2, AAV2.7m8, AAV2/5, or AAV2/8 vector. Suitable AAV vectors are described in, for example, U.S. Pat. No. 7,056,502 and Yan et al. (2002) J. Virology 76 (5): 2043-2053, the entire contents of which are incorporated herein by reference.

Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

Vectors suitable for use in the compositions and methods of the invention include those described in, for example, U.S. Patent Publicaton Nos. US2018/0245073A1, 2021/0207167, and 2022/0096657, the entire contents of each of which are incorporated herein by reference.

### VII. Delivery Methods of the Invention

The delivery of a nucleic acid molecule, i.e., a universal iRNA, a REVERSIR compound, or an expression vector as described herein to a cell e.g., a cell within a subject, such as a human subject (e.g., a subject in need thereof) can be achieved in a number of different ways. For example, delivery may be performed by contacting a cell with a universal iRNA, a REVERSIR compound, or an expression vector as described herein either in vitro or in vivo. In vivo delivery may also be performed directly by administering a composition comprising a universal iRNA and/or a REVERSIR compound as described herein to a subject. Alternatively, in vivo delivery may be performed indirectly by administering one or more vectors that encode and direct the expression of the universal iRNA, transgene, and/or REVERSIR compound. These embodiments are discussed further below.

For delivery of an expression vector encoding a transgene and comprising a universal iRNA target site or dsRNA agent targeting the transgene as described herein, any suitable non-viral, e.g., transfection, or viral, e.g., packaging in a viral particle and infection of a cell with the viral particle, means of delivery, e.g., administration to a subject may be used.

For example, a viral expression vector, e.g., an AAV expression vector, may be packaged into viral particles for use in the methods described herein, to produce viral vector particles using methods known in the art. Such methods generally include packaging the viral expression vectors of the invention into infectious viral particles in a host cell.

The expression vectors may be introduced into a host cell using any suitable method well known in the art. See Ausubel F, et al, Eds., “Short Protocols in Molecular Biology”, 4th Ed. (John Wiley and Sons, Inc., New York, NY, US, 1997), Brown (1995), Watson (1992), Alberts (2008), Innis (1990), Erlich (1989), Sambrook (1989), Bishop (1987), Reznikoff (1987), Davis (1986), and Schleef (2001), supra. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. When, for example, the expression vector is an AAV vector and the cell lacks the expression of any of the AAV rep and cap genes and genes providing adenoviral helper functions, the genes can be introduced into the cell simultaneously with the AAV vector. Alternatively, the genes can be introduced in the cell before or after the introduction of the expression vector as described herein.

Methods of culturing packaging cells and exemplary conditions which promote the release of viral vector particles, such as the producing of a cell lysate, are known in the art. Producer cells are grown for a suitable period of time in order to promote release of viral vectors into the media. Generally, cells may be grown for about 24 hours, about 36 hours, about 48 hours, about 72 hours, about 4 days, about 5 days, about 6 days, about 7 days, about 8 days, about 9 days, up to about 10 days. After about 10 days (or sooner, depending on the culture conditions and the particular producer cell used), the level of production generally decreases significantly. Generally, time of culture is measured from the point of viral production.

The viral vector particles can be obtained from both: i) the cells transfected with the foregoing and ii) the culture medium of the cells after a period of time post-transfection, preferably 72 hours. Any method for the purification of the viral vector particles from the cells or the culture medium can be used for obtaining the viral vector particles of the invention. Purified viral vector particles can be dialyzed against an appropriate formulation buffer such as PBS, filtered and stored at −80° C. Titers of viral genomes can be determined by quantitative PCR.

In some embodiments, the further purification steps, such as treatment of the cell lysate with benzonase, purification of the cell lysate with the use of affinity chromatography and/or ion-exchange chromatography are employed. See Halbert C, et al, Methods Mol. Biol. 2004; 246:201-212, Nass S, et al., Mol Ther Methods Clin Dev. 2018 Jun. 15; 9:33-46.

For delivery of nucleic acid molecules, e.g., REVERSIR and universal dsRNA agents, in general, any method of delivering a nucleic acid molecule (in vitro or in vivo) can be adapted for use with the present invention (see e.g., Akhtar S. and Julian R L. (1992) Trends Cell. Biol. 2 (5): 139-144 and WO94/02595, which are incorporated herein by reference in their entireties). For in vivo delivery, factors to consider include, for example, biological stability of the delivered molecule, prevention of non-specific effects, and accumulation of the delivered molecule in the target tissue. RNA interference has also shown success with local delivery to the CNS by direct injection (Dorn, G., et al. (2004) Nucleic Acids 32: e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602). Modification of the RNA or the pharmaceutical carrier can also permit targeting of the iRNA to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178).

In an alternative embodiment, delivery can include use of drug delivery systems such as a nanoparticle, a dendrimer, a polymer, liposomes, or a cationic delivery system. Positively charged cationic delivery systems which facilitate binding of an nucleic acid molecule (negatively charged) and also enhance interactions at the negatively charged cell membrane to permit efficient uptake by the cell. Cationic lipids, dendrimers, or polymers can either be bound to a nucleic acid, or induced to form a vesicle or micelle (see e.g., Kim S H, et al (2008) Journal of Controlled Release 129 (2): 107-116) that encases an iRNA. The formation of vesicles or micelles further prevents degradation of the nucleic acid when administered systemically. Methods for making and administering cationic-nucleic acid complexes are well within the abilities of one skilled in the art (see e.g., Sorensen, D R, et al (2003) J. Mol. Biol 327:761-766; Verma, U N, et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N, et al (2003), supra), “solid nucleic acid lipid particles” (Zimmermann, T S, et al (2006) Nature 441:111-114), cardiolipin (Chien, P Y, et al (2005) Cancer Gene Ther. 12:321-328; Pal, A, et al (2005) Int J. Oncol. 26:1087-1091), polyethyleneimine (Bonnet M E, et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A, et al (2007) Biochem. Soc. Trans. 35:61-67; Yoo, H., et al (1999) Pharm. Res. 16:1799-1804). In some embodiments, a nucleic acid forms a complex with cyclodextrin for systemic administration. Methods for administration and pharmaceutical compositions of nucleic acid and cyclodextrins can be found in U.S. Pat. No. 7,427,605, which is herein incorporated by reference in its entirety.

### VIII. Pharmaceutical Compositions of the Invention

The present invention also includes pharmaceutical compositions and formulations which include the universal iRNAs and/or REVERSIR compounds of the invention. In one embodiment, provided herein are pharmaceutical compositions containing a universal iRNA or REVERSIR compound, as described herein, and a pharmaceutically acceptable carrier. The pharmaceutical compositions containing the universal iRNA or REVERSIR compound are useful for treating a subject in need thereof. Such pharmaceutical compositions are formulated based on the mode of delivery. One example is compositions that are formulated for systemic administration via parenteral delivery, e.g., by subcutaneous (SC), intramuscular (IM), or intravenous (IV) delivery.

In some embodiments, the pharmaceutical compositions of the invention are sterile. In another embodiment, the pharmaceutical compositions of the invention are pyrogen free.

The pharmaceutical compositions of the invention may be administered in dosages sufficient to inhibit expression of a universal target sequence or universal dsRNA agent. In general, a suitable dose of the invention will be in the range of about 0.001 to about 200.0 milligrams per kilogram body weight of the recipient per day, generally in the range of about 1 to 50 mg per kilogram body weight per day. Typically, a suitable dose of an iRNA of the invention will be in the range of about 0.1 mg/kg to about 5.0 mg/kg, such as, about 0.3 mg/kg and about 3.0 mg/kg. A repeat-dose regimen may include administration of a therapeutic amount of iRNA on a regular basis, such as every month, once every 3-6 months, or once a year. In certain embodiments, administration is about once per month to about once per six months.

After an initial treatment regimen, the treatments can be administered on a less frequent basis. Duration of treatment can be determined based on the severity of disease.

In other embodiments, a single dose of the pharmaceutical compositions can be long lasting, such that doses are administered at not more than 1, 2, 3, or 4 month intervals. In some embodiments of the invention, a single dose of the pharmaceutical compositions of the invention is administered about once per month. In other embodiments of the invention, a single dose of the pharmaceutical compositions of the invention is administered quarterly (i.e., about every three months). In other embodiments of the invention, a single dose of the pharmaceutical compositions of the invention is administered twice per year (i.e., about once every six months).

The skilled artisan will appreciate that certain factors can influence the dosage and timing required to effectively treat a subject, including but not limited to mutations present in the subject, previous treatments, the general health or age of the subject, and other diseases present. Moreover, treatment of a subject with a prophylactically or therapeutically effective amount, as appropriate, of a composition can include a single treatment or a series of treatments.

The universal iRNA or REVERSIR compound can be delivered in a manner to target a particular tissue (e.g., hepatocytes).

Pharmaceutical compositions of the present invention include, but are not limited to, solutions, emulsions, and liposome-containing formulations. These compositions can be generated from a variety of components that include, but are not limited to, preformed liquids, self-emulsifying solids, and self-emulsifying semisolids. Formulations include those that target the liver.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers.

### A. Additional Formulations

i. Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution either in the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N. Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N. Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic, and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives, and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

The application of emulsion formulations via dermatological, oral, and parenteral routes, and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

ii. Microemulsions

In one embodiment of the present invention, the compositions are formulated as microemulsions. A microemulsion can be defined as a system of water, oil, and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215).

iii. Microparticles

A universal iRNA or REVERSIR compound of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p.92). Each of the above mentioned classes of penetration enhancers and their use in manufacture of pharmaceutical compositions and delivery of pharmaceutical agents are well known in the art.

v. Excipients

In contrast to a carrier compound, a “pharmaceutical carrier” or “excipient” is a pharmaceutically acceptable solvent, suspending agent, or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Such agent are well known in the art.

vi. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings, or aromatic substances, and the like which do not deleteriously interact with the nucleic acid(s) of the formulation.

Aqueous suspensions can contain substances which increase the viscosity of the suspension including, for example, sodium carboxymethylcellulose, sorbitol, or dextran. The suspension can also contain stabilizers.

Toxicity and prophylactic efficacy of such compounds can be determined by standard pharmaceutical procedures in cell cultures or experimental animals, e.g., for determining the LD50 (the dose lethal to 50% of the population) and the ED50 (the dose prophylactically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED50, such as, an ED80 or ED90, with little or no toxicity. The dosage can vary within this range depending upon the dosage form employed and the route of administration utilized. For any compound used in the methods featured in the invention, the prophylactically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) or higher levels of inhibition as determined in cell culture. Such information can be used to more accurately determine useful doses in humans. Levels in plasma can be measured, for example, by high performance liquid chromatography.

In addition to their administration, as discussed above, the iRNAs and/or REVERSIR compounds featured in the invention can be administered in combination with other known agents used for the prevention or treatment of a disease of interest. In any event, the administering physician can adjust the amount and timing of iRNA administration on the basis of results observed using standard measures of efficacy known in the art or described herein.

### VI. Methods of the Invention

The present invention also provides methods of using the universal dsRNA agents and REVERSIR compounds of the invention.

In one aspect, the present invention provides a method of modulating expression of a transgene in a cell. The method includes contacting the cell with an expression vector encoding a transgene and comprising a universal iRNA target site; contacting the cell with a universal double stranded ribonucleic acid (dsRNA) agent which recognizes and binds to the universal iRNA target site to thereby inhibit expression of the transgene, thereby inhibiting the expression of the transgene; and, optionally, further contacting the cell with a REVERSIR compound that abrogates the iRNA activity of the universal dsRNA agent, thereby allowing expression of the transgene.

Contacting of a cell may be done in vitro or in vivo. Contacting a cell in vivo includes contacting a cell or group of cells within a subject, e.g., a human subject. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the RNAi agent and/or REVERSIR compound to a site of interest.

As used herein, the various forms of the term “modulate” are intended to include stimulation (e.g., increasing or upregulating a particular response or activity) and inhibition (e.g., decreasing or downregulating a particular response or activity).

The term “inhibiting,” as used herein, is used interchangeably with “reducing,” “silencing,” “downregulating”, “suppressing”, and other similar terms, and includes any level of inhibition.

“Inhibiting expression” includes any level of inhibition, e.g., at least partial suppression of the expression. The expression may be assessed based on the level, or the change in the level, of any variable associated with the transgene, e.g., mRNA level or protein level. This level may be assessed in an individual cell or in a group of cells, including, for example, a sample derived from a subject.

Inhibition may be assessed by a decrease in an absolute or relative level of one or more variables that are associated with expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the invention, expression of a transgene is inhibited by at least 50%, 55%, 60%, 65%, 70%, 75%, 80%, 85%, 90%, or 95%, or to below the level of detection of the assay. In some embodiments, expression of a transgene is inhibited by at least 70%. It is further understood that inhibition of transgene expression in certain tissues may be desirable.

Inhibition of the expression of a transgene may be manifested by a reduction of the amount of mRNA expressed by a first cell or group of cells as compared to a second cell or group of cells substantially identical to the first cell or group of cells.

In other embodiments, inhibition of the expression of a transgene may be assessed in terms of a reduction of a parameter that is functionally linked to transgene expression, e.g., transgene protein level in blood or serum from a subject. Transgene silencing may be determined in any cell expressing the transgene, either endogenous or heterologous from an expression construct, and by any assay known in the art.

Inhibition of the expression of a transgene protein may be manifested by a reduction in the level of the transgene protein that is expressed by a cell or group of cells or in a subject sample (e.g., the level of protein in a blood sample derived from a subject). As explained above, for the assessment of mRNA suppression, the inhibition of protein expression levels in a treated cell or group of cells may similarly be expressed as a percentage of the level of protein in a control cell or group of cells, or the change in the level of protein in a subject sample, e.g., blood or serum derived therefrom.

The level of transgene mRNA that is expressed by a cell or group of cells may be determined using any method known in the art for assessing mRNA expression. In one embodiment, the level of expression of transgene in a sample is determined by detecting a transcribed polynucleotide, or portion thereof, e.g., mRNA of the transgene gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression of transgene is determined using a nucleic acid probe. The term “probe”, as used herein, refers to any molecule that is capable of selectively binding to a specific transgene. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to transgene mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of transgene mRNA.

An alternative method for determining the level of expression of transgene in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression is determined by quantitative fluorogenic RT-PCR (i.e., the TaqMan™ System).

The expression levels of transgene mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of transgene expression level may also comprise using nucleic acid probes in solution.

In some embodiments, the level of mRNA expression is assessed using branched DNA (bDNA) assays or real time PCR (qPCR). The use of these methods is described and exemplified in the Examples presented herein. In some embodiments, expression level is determined by the method provided in Example 2 using a 10 nM siRNA concentration in the species matched cell line.

The level of transgene protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the invention are assessed by a decrease in transgene mRNA or protein level (e.g., in a liver biopsy).

In another aspect, the present invention provides a method of treating a subject in need thereof. The methods include contacting an expression vector encoding a therapeutic transgene and comprising a universal iRNA target site administered to the subject with a universal double stranded ribonucleic acid (dsRNA) agent which recognizes and binds to the universal iRNA target site to thereby inhibit expression of the transgene, thereby treating the subject.

In some embodiments, the universal dsRNA agent is further contacted with a REVERSIR compound that abrogates the iRNA activity of the universal dsRNA agent to thereby allow the expression of the transgene.

In one aspect, the present invention provides a method of treating a subject in need thereof. The method include contacting an expression vector encoding a therapeutic transgene and a double stranded ribonucleic acid (dsRNA) agent targeting the transgene administered to the subject with a REVERSIR compound that abrogates the iRNA activity of the dsRNA agent to thereby allow expression of the transgene, thereby treating the subject.

In another aspect, the present invention provides a method of treating a subject in need thereof. The method include administering to said subject an expression vector encoding a transgene and comprising a universal iRNA target site; allowing expression of the transgene until a desired level of expression has been achieved; and once a desired level of expression of the transgene has been achieved, administering to said subject a universal double stranded ribonucleic acid (dsRNA) agent, comprising a sense strand and an antisense strand forming a double stranded region, which recognizes and binds to the universal iRNA target site to thereby inhibit the expression of the transgene, thereby treating said subject.

In some embodiment, the method further comprises administering to said subject a REVERSIR compound once the level of the transgene has dropped below a desired level of expression, wherein the REVERSIR compound abrogates the iRNA activity of the universal dsRNA agent to thereby allow the expression of the transgene.

The in vivo methods of the invention may include administering to a subject a composition by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intramuscular injection.

An compositions of the invention may be administered as a “free iRNA” or a “free REVERSIR compound.” A free iRNA or free REVERSIR compound is administered in the absence of a pharmaceutical composition. The naked iRNA or naked REVERSIR compound may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, an composition of the invention may be administered as a pharmaceutical composition, such as a liposomal formulation.

“Subjects in need thereof” include subjects having a genetic disorder that have been, or are to be, treated with a system as described herein. Genetic disorders that may be treated using the methods of the invention include any diseases and disorders described on the website of the National Institutes of Health under the topic subsection Genetic Disorders (website at health.nih.gov/topic/GeneticDisorders), such as blood and coagulation diseases and disorders; bleeding disorders; cell dysregulation and oncology diseases and disorders; autoimmune diseases; metabolic, liver, kidney and protein diseases and disorders; muscular/skeletal diseases and disorders; neurological and neuronal diseases and disorders; occular diseases and disorders; epilepsy; Duchenne muscular dystrophy; schizophrenia; trinucleotide repeat disorders; Fragile X Syndrome; secretase related disorders; prion-related disorders; drug addiction, autism; Alzheimer's disease; Parkinson's disease; Down Syndrome; Cystic Fibrosis; Thalassemia; Sickle Cell Anemia; Huntington's Disease; and Tay-Sachs Disease.

Exemplary disorders that may be treated using the methods of the invention include any of the diseases and disorders described in US2019/0153471, the entire contents of which are incorporated herein by reference, including, for example, cancers, autoimmune and immune system disorders, ocular diseases, nervous system diseases, inflammations, and infections, amongst many others.

The methods of the invention may be practiced in combination with other pharmaceuticals and/or other therapeutic methods, e.g., with known pharmaceuticals and/or known therapeutic methods, such as, for example, those which are currently employed for treating the disorders described herein.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1. Materials and Methods

The following materials and methods were used in the Examples below.

### Plasmids

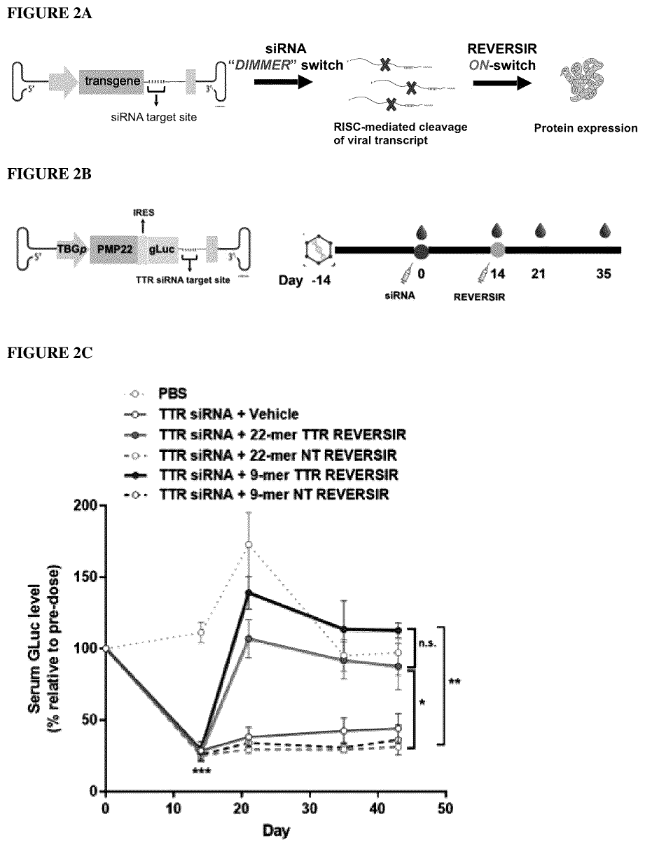

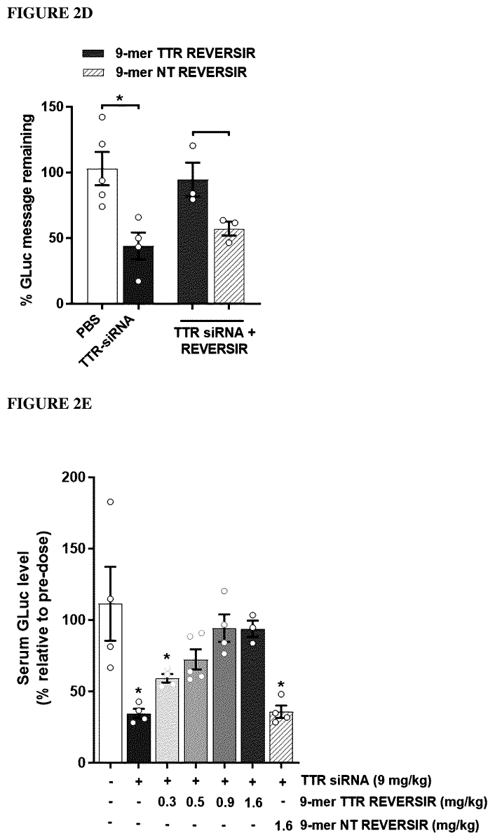

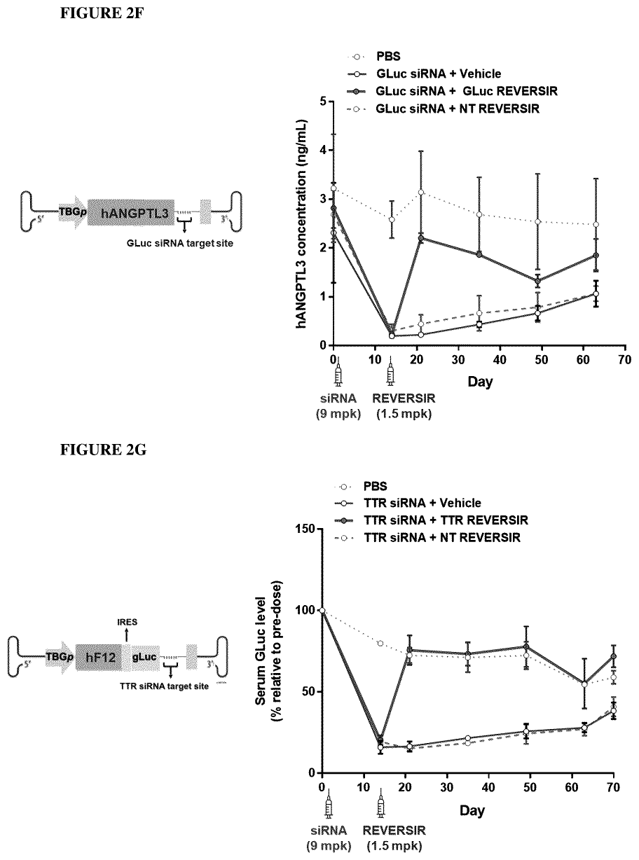

On- and off-target reporters were generated by sub-cloning a DNA fragment containing a fully complementary (23-mer) or partial siRNA target sites into the psiCHECK2 vector (Promega C8021) between Xho1 and Not1 restriction sites. Off-target reporters incorporated either 1 seed-matched site or 4 tandem seed-matched sites, complementary to antisense positions 2 to 9. rAAV vectors were generated using Vector Builder's or Blue Heron's single-stranded AAV vector backbone with a TBG promoter, KOZAK sequence preceding the transgene, and bGH-poly A signal. For vectors harboring pri-miRNA-adapted shRNAs, the chimeric intron cassette from pAAVsc-CB-PI-GLuc was sub-cloned between the promoter and transgene elements, and the shRNA fragment was cloned into the PpuM1 site. The miR-33 and miR-30E shRNA sequences were designed as described previously. A fully matched siRNA target site was inserted immediately downstream of the GLuc transgene, without any intervening spacer sequence. rAAV vectors used in FIG. 2 expressed a mono- or bicistronic transcript with a fully complementary siRNA target site after the transgene stop codon, separated by a NotI restriction site.

### Care and Use of Laboratory Animals

All procedures and protocols performed on mice adhered to care guidelines approved by the Institutional Animal Care and Use Committee (IACUC) at Alnylam and were compliant with local, state, and federal regulations. Female C57BL/J mice and male Sprague Dawley rats between 6-8 weeks of age were obtained from Charles River Laboratories and allowed to acclimate in-house for 48 hours prior to initiation of studies. Adult mice were group housed (up to 5 sex-matched animals per cage) on a standard 12:12 hour light-dark cycle and provided access to food and water ad libitum.

Mouse AAV studies: Single-stranded rAAV8 vectors were generated, purified, and titered either at University of Massachusetts Viral Vector Core or Signagen Laboratories. Viral stocks were diluted in sterile PBS, and administered intravenously by tail vein injection at the indicated titer in a total volume of 100 μL. Two weeks after AAV administration, mice were subcutaneously injected with siRNA or REVERSIR, or phosphate buffered saline (PBS) as control, in a total dosing volume of 10 μL/g. All siRNA and Reversir test articles were diluted to the appropriate dosing concentration in PBS. Animals were sacrificed at the indicated days, after which terminal livers were harvested, flash frozen in liquid nitrogen, and stored at −80° C. until further downstream analysis.

Rat toxicity studies: Test articles were diluted with 0.9% NaCl to achieve appropriate dosing concentrations and dosed subcutaneously on the upper back to male Sprague Dawley rats in a dose volume of 5 mL/kg with N=3 animals/group. Rats were sacrificed and livers and blood collected on Day 16. Randomization was performed using the partitioning algorithm in the Pristima® Suite (Xybion) that avoids group mean body weight bias. Investigators were not blinded to the group allocation during the experiment or when assessing the outcome.

### Clinical Pathology

Whole-venous blood was collected into serum separator tubes (BD Microtainer) and allowed to clot at room temperature for 30 minutes prior to centrifugation at 3000 RPM (1489×g) for 10 minutes at 4° C. Serum was then aliquoted and stored at −80° C. until analyses. Serum chemistries were analyzed using the AU400 chemistry analyzer (Beckman Coulter-Brea, CA, USA), with reagents provided by Beckman Coulter, Randox, and Sekisui Diagnostics.

### Histopathology

All animals were sacraficed as per Alnylam standard operating procedures and tissues of interest were collected. All tissues were fixed in 10% neutral buffered formalin (10% NBF) for 72 hours prior to routine processing using TissueTek VIP 6A1 (Sakura). Tissues were trimmed, embedded into paraffin blocks, sectioned at 4μ, stained with Hematoxylin and Eosin (H&E) using TissueTek Prisma A1D (Sakura), and coverslipped using TissueTek Glass g2 (Sakura). Two sections were examined microscopically from each liver in an un-blinded fashion, followed by blinded assessment to confirm subtle findings. The range of severity grade for each histologic finding was graded on a scale of 1-5 with 1 indicating minimal severity and 5 indicating highest severity.

### Oligonucleotide Synthesis

All oligonucleotides were synthesized on an MerMade 192 or MerMade 12 synthesizer according to previously published protocols.

Bioinformatic Prediction of Transgene Regulator siRNA Sequences

Selection of the molecular switch duplexes was informed by conventions used for therapeutic siRNA development candidates.

A set of all decamers was generated in silico to represent the first 10 bases of a candidate guide sequence (1,048,576 sequences). miRNA seed sequences were retrieved from the miRbase database for Homo sapiens, Mus musculus, Rattus norvegicus, Macaca mulatta and Macaca nemestris. The non-human primate species were selected as proxies for Macaca fascicularis (cynomolgus monkey), which is not represented in miRbase. Decamers containing seed sequences (bases 2-7) matching the miRNA seed sequences were removed from further consideration (487,186 decamers remaining).

Each remaining decamer was annotated with a predicted quiescence score based on a proprietary regression model derived from analysis of shRNA perturbagen data. Those with scores in the lowest quartile (predicted most quiescent) were retained (155,374 decamers), and others removed from consideration.

The frequency of heptamers found in the human transcriptome (NCBI RefSeq), as was computed by aligning transcripts for each gene, and counting all heptamer substrings in the global alignment consensus and non-overlapping regions. Decamers were then annotated with the frequency of the heptamer in the seed (positions 2-8), and those in the lowest decile heptamer frequency were retained (1,767 decamers) and the remainder removed from consideration.

The remaining decamers were then aligned to the human, mouse, rat, and cynomolgus monkey transcriptomes using BLASTN with the parameters “-task blastn-short-dust no -evalue 1000 -ungapped -perc_identity 100” to count the number of occurrences of each decamer within each transcriptome. The decamers were then sorted in ascending order based on the total number of identical alignments on the reverse complement strand, then predicted quiescence, and number of seed matches in the human transcriptome.

For each decamer, 10,000 random 13-mer sequences were created and appended to create 10,000 candidate 23-mer siRNA guide sequences with a common 10-mer prefix. Each 23-mer was then aligned to the transcriptomes of human, mouse, rat, and cynomolgous monkey using a weighted ungapped alignment of the 23-mer to the transcripts, (mismatch penalty for positions 2-9 is 2.8, for positions 10-11, 1.2, for positions 12-19, 1.0, and for positions 1 and 20-23, 0.0). For each candidate decamer prefix, the 23-mers with the top 10 worst alignment profiles (most poorly aligned to the transcriptomes) were retained. Sequences were sorted by their alignment score and predicted quiescence, and top candidates were selected.

### Serum and Plasma Collection

Blood was collected by retro-orbital bleeding under isoflurane anesthesia in accordance with IACUC approved protocols. Approximate 250 μL of blood was collected once per week from one eye, with subsequent bleeds using alternate eyes. For serum samples, blood was collected in Becton Dickinson serum separator tubes (Fisher Scientific, BD365967), kept at room temperature for 1 hour and then spun in a micro-centrifuge at 21,000×g at room temperature for 10 minutes. For plasma samples, blood was collected in Becton Dickinson plasma (K2EDTA) separator tubes (Fisher Scientific, BD365974), kept on ice for 30 minutes before being centrifuged at 10,000×g at 4° C. for minutes. Both serum and plasma samples were aliquoted and transferred to 96-well plates for 10 storage at −80° C.

### ELISA Assays

Circulating AAV-expressed human ANGPTL3 protein levels were measured from plasma using commercially-available ELISA kits (plasma diluted 1:4 and used with R&D Systems #DANL30). The assay is specific for detection of human ANGPTL3 protein, with no significant cross-reactivity to other related angiopoietin molecules or mouse ANGPTL3. Mouse EPO concentrations were measured with Mouse EPO Quantikine ELISA kit from R&D Systems MEPOOB (serum diluted 1:2000). TTR protein levels were measured with mouse prealbumin kit from ALPCO, 41-PALMS-E01 (serum diluted 1:4000). All assays were performed following the manufacturer's protocols.

### Cell Lines and Transfection

Cos-7 (ATCC CRL-1651) and HepG2 (ATCC HB-8065) cells were grown in DMEM and EMEM, respectively, both supplemented with 10% heat-inactivated FBS and 1% glutamine and maintained in a humidified incubator at 37°, 5% CO₂. Plasmids and siRNAs were co-delivered by reverse transfection using Lipofectamine 2000 (Thermo Fisher Scientific 11668) for Cos-7 cells and Lipofectamine 3000 for HepG2 cells, following the manufacturer's protocol.

### Luciferase Reporter Assays

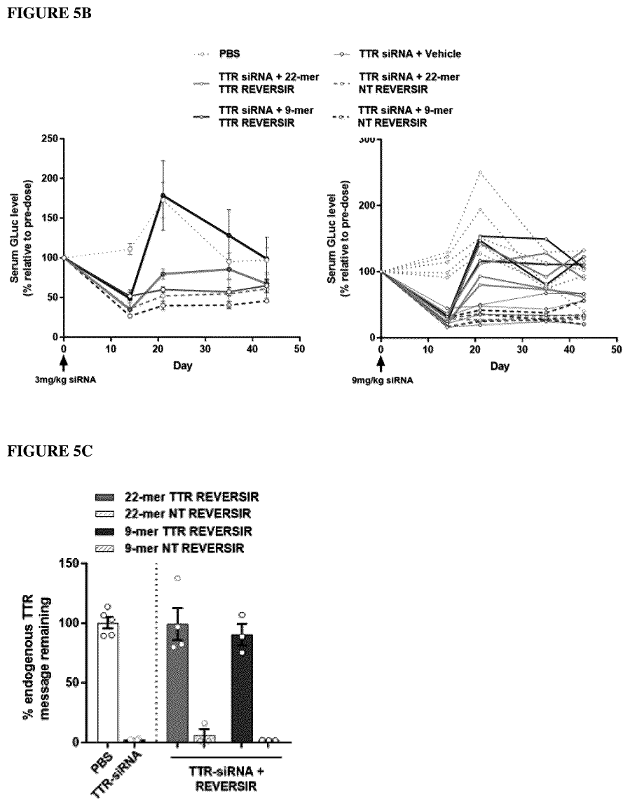

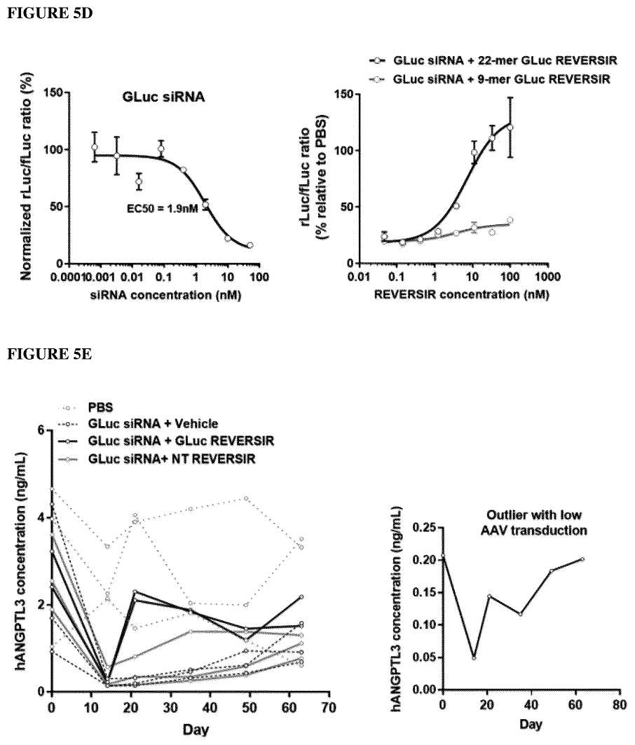

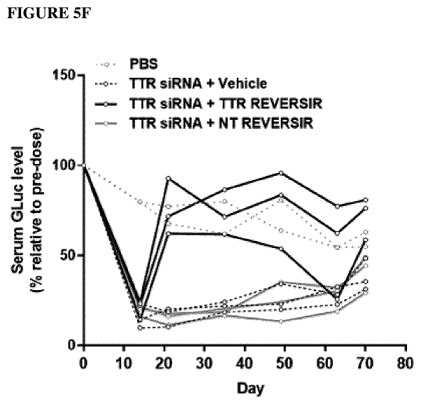

siRNA on-target and off-target reporter evaluations: Cos7 cells were co-transfected with 5 ng psiCHECK2 reporter plasmid and the specified amounts of siRNA duplexes (serially diluted in PBS) in a 384-well plate format at a density of 5×10³ cells per well. To assess REVERSIR-mediated rescue of knockdown by vector-encoded shRNAs (FIG. 5), Cos7 cells were co-transfected in the same 384-well format with 70 ng of shRNA expression plasmid, 5 ng of psiCHECK2 reporter, in addition to indicated amounts of the specified REVERSIR molecules (serially diluted in PBS). Forty-eight hours post-transfection, Firefly (transfection control) and Renilla (target) luciferase activities were sequentially measured using the Dual-Glo Luciferase Assay System (Promega E2920) and detected on a Spectramax M plate reader (Molecular Devices). The Renilla signal was normalized to Firefly signal for each well and expressed as a percentage relative to control wells transfected with reporter alone without siRNA or non-targeting shRNA plasmid. All transfections were performed at least in triplicate.

In vitro characterization of self-silencing AAV constructs: HepG2 cells were seeded at a density of 2×10⁴ cells per well in a 96-well plate and co-transfected with 16.6 ng intronic shRNA-containing GLuc expression plasmid along and 20 nM or 40 nM REVERSIR. As an internal control to normalize for transfection efficiency, 3.3 ng of PGK-driven Luc2 (pGL4.53[luc2/PGK]) vector was also co-transfected, constituting 17% of the total transfected DNA. Reverse transfections were carried out with 0.1 μL P3000 and 0.2 μL Lipofectamine 3000 per well and allowed to proceed for 6 hours after which the media was replaced. Expression of GLuc and Luc2 reporters was measured 48 hours after transfection. To measure secreted GLuc levels, cell culture supernatant from each sample was diluted 1:50 in EMEM. 5 μL of diluted supernatant and 50 μL of assay buffer containing 3 μM coelenterazine substrate (Selleck Chem S7777; stocks made up to 1 mM in DMSO and subsequently diluted to 3 μM in PBS) were transferred to each well of a white opaque 96-well plate and read on a Spectramax L microplate luminometer. Plate was dark-adapted to minimize auto-luminescence and injection speed was set to 250 μL/s, followed by 2 s shake and 1 second signal integration time per well. To determine cellular Luc2 expression, cells in each well were first lysed with 50 μL ice-cold 1× passive lysis buffer (Promega E1941), allowed to incubate on an orbital shaker for 10 minutes at room temperature, after which 50 μL of ONE-Glo EX luciferase reagent (Promega E8110) was added. Luciferase signals were detected on a SpectraMax M plate reader 5 minutes later. The ratio of GLuc to FLuc intensity was computed for each well, and expressed as percent normalized to a matched NT shRNA-expressing condition.

Longitudinal monitoring of secreted GLuc levels in vivo: Five μL of serum was assayed as described above on a Spectramax L luminometer. All serum samples corresponding to a study were run simultaneously under uniform conditions and resulting GLuc signals at an individual timepoint were expressed as percent relative to that at D0 (pre-treatment with siRNA or REVERSIR) for each animal.

### RNA Isolation and RT-qPCR Evaluations

96-well plates of cells were directly homogenized in 100 μL RLT buffer (Qiagen) containing 10 μL/mL beta-mercaptoethanol and allowed to incubate on an orbital shaker for 10 minutes at room temperature. RNA was extracted using the Qiagen RNeasy 96 kit (#74181), with additional incorporation of an on-column DNase digestion step (Qiagen 79254). For in vivo samples, powdered liver (˜10 mg) was resuspended in 900 μl QIAzol (RNeasy 96 Universal Tissue Kit, Qiagen, 74881) and homogenized at 25/seconds for 1 minute at 4° C. using a TissueLyser II (Qiagen, 85300). Samples were incubated at room temperature for 5 minutes followed by addition of 180 μl chloroform. Samples were vigorously mixed, followed by a 10 minute incubation at room temperature. Samples were spun at 12,000×g for 15 minutes at 4° C., the supernatant was moved to a new tube, and 1.5 volumes of 70% ethanol was added. Samples were then purified using a RNeasy 96 Universal Tissue Kit (Qiagen, 74881) with on-column DNase digestion. RNA was eluted from the RNeasy spin columns with 50 μl RNAse-free water (Ambion) and quantified on a Nanodrop (Thermo Fisher Scientific). 0.5 μg of purified RNA was reverse transcribed using either the iScript gDNA Clear cDNA Synthesis Kit (BioRad, #1725034) or Maxima H Minus First Stand cDNA Synthesis Kit (Thermo Fisher Scientific M1681). RNA was DNase-treated prior to cDNA synthesis and oligo (dt) 18 primers were use, where applicable. The product was diluted 1:2 in RNase-free water and subjected to quantitative real-time PCR (qRT-PCR) using gene-specific TaqMan assays (Thermo Fisher Scientific 4331182) for mouse TTR (Mm00443267_m1), human PMP22 (Hs00165556_m1), Luc2 (custom probe), and GLuc (custom probe). Levels of mouse (Mm99999915_g1) or human (Hs99999905_m1) GAPDH were used as endogenous normalization controls. Real-Time PCR was performed in a Roche LightCycler 480 using LightCycler 480 Probes Master Mix (Roche, 04707494001). No-reverse transcriptase enzyme controls were performed on a few samples in each study to ensure lack of viral or genomic DNA contamination. All RT-qPCR data were analyzed using the ΔΔCt method.

### RNA-Seq Methods

Primary Mouse Hepatocytes (BIOIVT, Cat #M005052-P, Lot GBW) were transfected in 384-well plates (5000 cells per well) with siRNA or DPBS (mock control) at a final concentration of 50 nM using Invitrogen Lipofectamine® RNAiMAX (Invitrogen, Carlsbad, CA, Catalog No. 13778-150). After 48 hours, cell were lysed in lysis buffer (Tris HCl pH 7.5 100 mM, LiCl 500 mM, EDTA pH 8.0 10 mM, LiDS 1%, DTT 5 mM supplemented with TURBO™ DNase, Thermofisher #AM2238) for 30 min. Subsequently whole transcriptome mRNA-enriched RNA-Seq libraries using a KAPA mRNA Capture Kit and mRNA HyperPrep Kit (Roche) were constructed from cell lysates as per protocol but in 5× miniaturized reagent scale (except mRNA capture beads were resuspended in water prior to mix with lysate). RNA-Seq libraries were quantified by low depth sequencing on Illumina iSeq instrument. Equal amounts of each library/sample were pooled and sequenced on a Illumina NovaSeq instrument with 2×150 bp paired-end settings, according to manufacturers' instructions. Raw RNAseq reads were filtered with minimal mean quality scores of 28 and minimal remaining length of 36, using the ea-utils software fastq-mcf v1.05 (33). Filtered reads were aligned to the Mus musculus genome (GRCm39/mm39) using STAR (ultrafast universal RNAseq aligner) v2.7.9a (34). Uniquely aligned reads were counted by featureCounts v2.0.2 (35) with the minimum mapping quality score set to 10. Differential gene expression analysis was performed by the R package DESeq2 v1.34.0 with the betaPrior parameter set to TRUE to shrink log 2 fold-change estimates for noisy, low-count genes (36).

### Statistical Analysis

Statistical analyses were performed using GraphPad Prism v.7 software. Data were analyzed by one-way or two-way ANOVA followed by Bonferroni's, Tukey's, or Holm-Sidak post hoc tests for multiple comparisons. In certain instances where indicated, individual unpaired two-tailed t-test or multiple t-testing (one per row) with Holm-Sidak correction was employed. All data are presented as mean+/−s.e.m. and differences between groups considered significant if ***p<0.0001, ***p<0.001, **p<0.01, or *p<0.05

TABLE 1
Abbreviations of nucleotide monomers used in nucleic acid sequence representation. It will be understood that these
monomers, when present in an oligonucleotide, are mutually linked by 5′-3′-phosphodiester bonds; and it is under-
stood that when the nucleotide contains a 2′-fluoro modification, then the fluoro replaces the hydroxy at that
position in the parent nucleotide (i.e., it is a 2′-deoxy-2′-fluoronucleotide). It is to be further understood that the
nucleotide abbreviations in the table omit the 3′-phosphate (i.e., they are 3′-OH) when placed at the 3′-terminal
position of an oligonucleotide.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O—methyladenosine-3′-phosphate |
| as | 2′-O—methyladenosine-3′-phosphorothioate |
| c | 2′-O—methylcytidine-3′-phosphate |
| cs | 2′-O—methylcytidine-3′-phosphorothioate |
| g | 2′-O—methylguanosine-3′-phosphate |
| gs | 2′-O—methylguanosine-3′-phosphorothioate |
| t | 2′-O—methyl-5-methyluridine-3′-phosphate |
| ts | 2′-O—methyl-5-methyluridine-3′-phosphorothioate |
| u | 2′-O—methyluridine-3′-phosphate |
| us | 2′-O—methyluridine-3′-phosphorothioate |
| s | phosphorothioate linkage |
| L10 | N-(cholesterylcarboxamidocaproyl)-4-hydroxyprolinol (Hyp-C6-Chol) |
| L96 | N-[tris(GalNAc-alkyl)-amidodecanoyl)]-4-hydroxyprolinol |
| | (Hyp-(GalNAc-alkyl)3) |
| | (2S,4R)-1-[29-[[2-(acetylamino)-2-deoxy-β-D-galactopyranosyl]oxy]-14,14-bis[[3-[[3-[[5-[[2-(acetyl- |
| | amino)-2-deoxy-β-D-galactopyranosyl]oxy]-1-oxopentyl]amino]propyl]amino]-3-oxopropoxy]methyl]- |
| | 1,12,19,25-tetraoxo-16-oxa-13,20,24-triazanonacos-1-yl]-4-hydroxy-2-hydroxymethylpyrrolidine |
| | 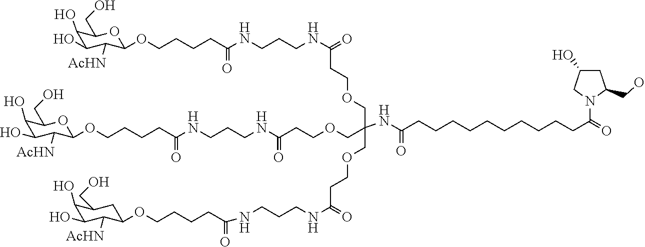 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)CC(CO)C(O)C1O] |
| uL96 | 2′-O—methyluridine-3′-phosphate ((2S,4R)-1-[29-[[2-(acetylamino)-2-deoxy-β-D-galactopyranos- |
| | yl]oxy]-14,14-bis[[3-[[3-[[5-[[2-(acetylamino)-2-deoxy-β-D-galactopyranosyl]oxy]-1-oxopentyl] |
| | amino]propyl]amino]-3-oxopropoxy]methyl]-1,12,19,25-tetraoxo-16-oxa-13,20,24-triazanonacos- |
| | 1-yl]-4-hydroxy-2-pyrrolidinyl)methyl ester |
| | 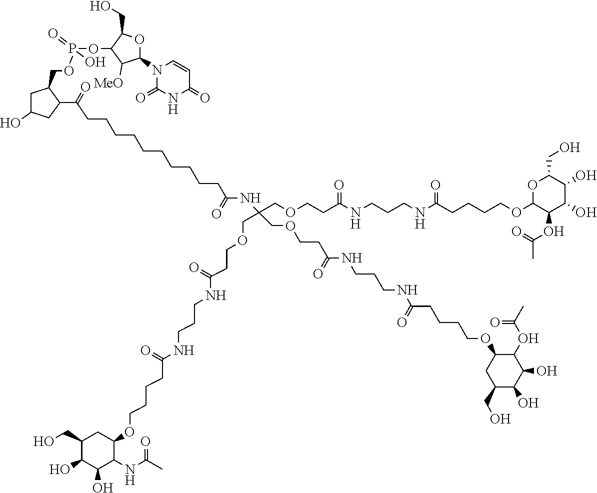 [SMILES: COC1C(OP(=O)(O)OC[C@@H]2CC(O)CC2C(=O)CCCCCCCCCCC(=O)NC(COCCC(=O)NCCCNC(=O)CCCCOC2O[C@H](CO)[C@H](O)[C@H](O)[C@H]2OC(C)=O)(COCCC(=O)NCCCNC(=O)CCCCO[C@@H]2C[C@H](CO)[C@H](O)[C@H](O)C2NC(C)=O)COCCC(=O)NCCCNC(=O)CCCCO[C@@H]2C[C@H](CO)[C@H](O)[C@H](O)C2OC(C)=O)[C@@H](CO)O[C@H]1n1ccc(=O)[nH]c1=O] |
| Y34 | 2-hydroxymethyl-tetrahydrofurane-4-methoxy-3-phosphate (abasic 2′-OMe furanose) |
| | 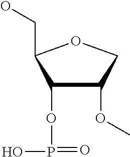 |
| Y44 | inverted abasic DNA (2-hydroxymethyl-tetrahydrofurane-5-phosphate) |
| | 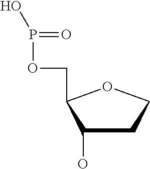 |
| L10 | N-(cholesterylcarboxamidocaproyl)-4-hydroxyprolinol (Hyp-C6-Chol) |
| | 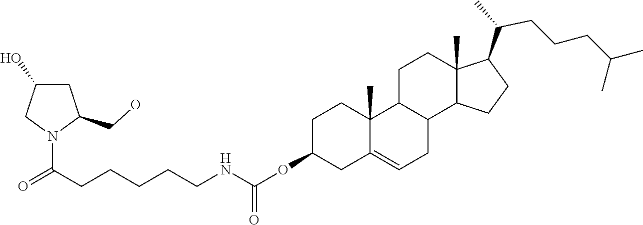 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Chd) | 2′-O—hexadecyl-cytidine-3′-phosphate |
| (Ahd) | 2′-O—hexadecyl-adenosine-3′-phosphate |
| (Ghd) | 2′-O—hexadecyl-guanosine-3′-phosphate |
| (Uhd) | 2′-O—hexadecyl-uridine-3′-phosphate |
| Q191s | N-[tris(GalNAc-alkyl)-amidododecanoyl]-(S)-pyrrolidin-3-ol-phosphorothioate(p-C12-(GalNAc-alkyl)3) |
| | 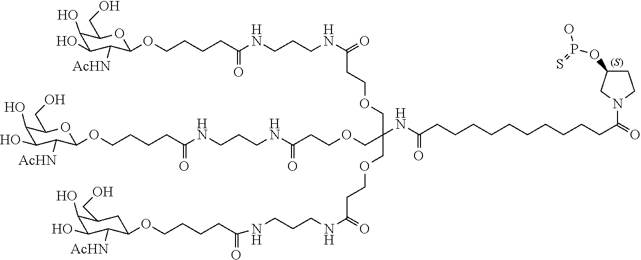 |
| (Aln) | 2′-O,4′-C—methylene adenosine-3′-phosphate |
| (Alns) | 2′-O,4′-C—methylene adenosine-3′-phosphorothioate |
| (m5Cln) | 2′-O,4′-C—methylene 5-methylcytidine-3′-phosphate |
| (m5Clns) | 2′-O,4′-C—methylene 5-methylcytidine-3′-phosphorothioate |
| (Gln) | 2′-O,4′-C—methylene guanosine-3′-phosphate |
| (Glns) | 2′-O,4′-C—methylene guanosine-3′-phosphorothioate |
| (Tln) | 2′-O,4′-C—methylene 5-methyluridine-3′-phosphate |
| (Tlns) | 2′-O,4′-C—methylene 5-methyluridine-3′-phosphorothioate |

### Example 2. RNAi-Mediated Regulatory Switch for Modulation of AAV-Delivered Transgenes In Vivo

Broader adoption of adeno-associated virus (AAV)-based vectors for gene delivery could be further facilitated by controllable approaches to fine-tune the magnitude and timing of expression of transgenes. Previously reported approaches for temporal and dosage control of gene therapies have been limited by poor dynamic ranges and timescales of transgene induction, a need for immunogenic protein components, or a lack of proven clinical translation. Therapeutics based on RNA interference (RNAi) hold unique potential for regulation of AAV transgene dosage as a clinically proven modality that utilizes a naturally occurring mechanism to modulate gene expression in a highly sequence-specific manner. As described herein, a generalizable RNAi-based molecular switch to pharmacologically modulate expression of AAV-delivered transgenes is presented. For silencing of the AAV transgene, either the clinically validated modality of chemically-modified short interfering RNA (siRNA) conjugates or intronically-encoded co-expression of short hairpin RNA shRNA from the AAV vector was employed. For induction of transgene expression in both settings, REVERSIR technology, a synthetic high-affinity single-stranded oligonucleotide complementary to RISC-loaded siRNA strand to block RNAi activity of exogenous siRNA or embedded shRNA was employ, thus affording dose-dependent and tunable duration of recovery of liver-directed AAV transgene expression in vivo. For clinical development, potent and highly specific siRNA sequences designed to have no homology to any annotated genes across human or mammalian preclinical transcriptomes that can be used to selectively regulate transgenes while minimizing unintended off-target effects were developed. Together, the results presented herein establish a framework for the use of RNAi-based regulatory switches with infrequent dosing in clinical settings to refine transgene dosage and timing of induction from exogenous vector delivery systems in a dynamic manner.

Temporal Regulation by REVERSIR-Mediated Induction of Transgene Under Control of shRNA

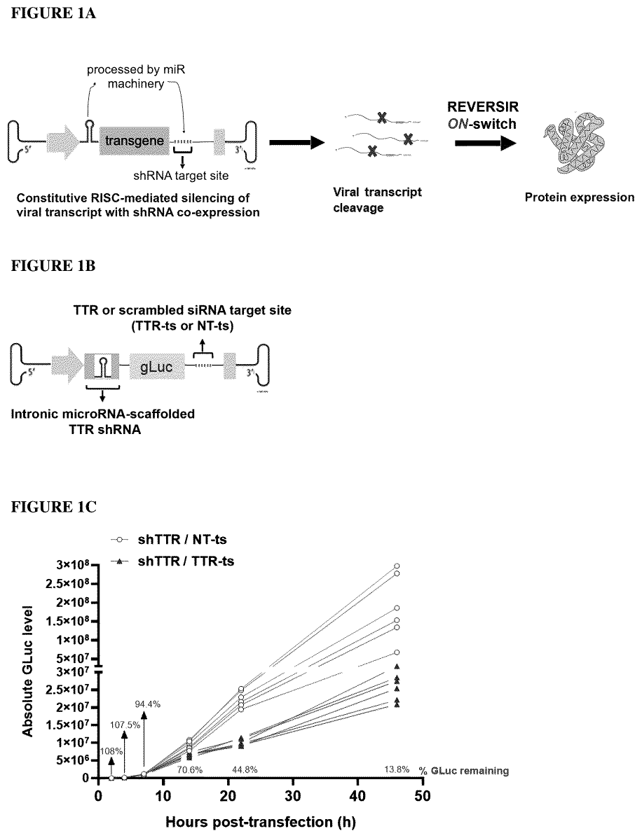

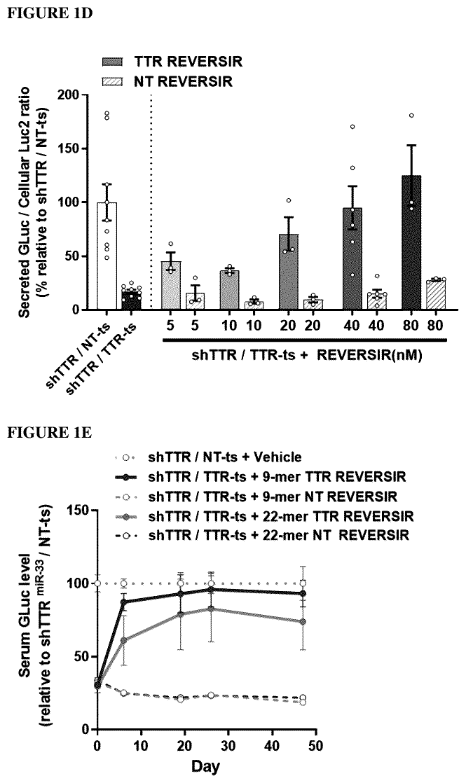

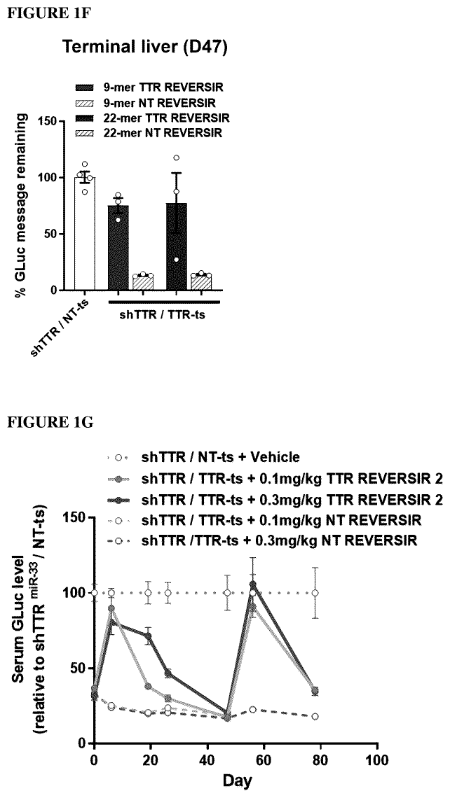

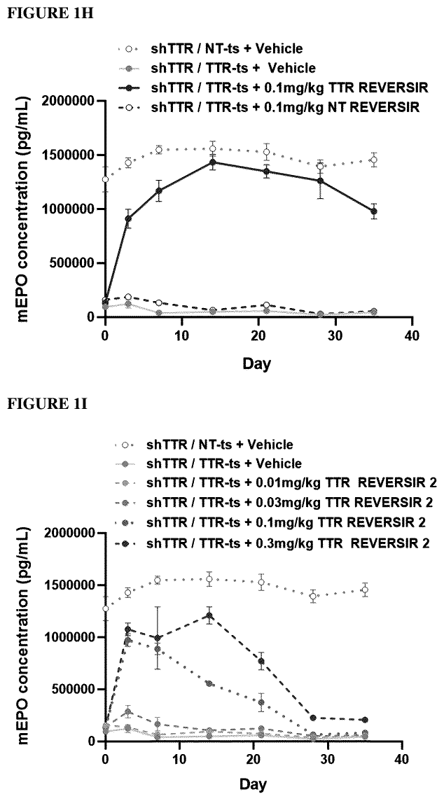

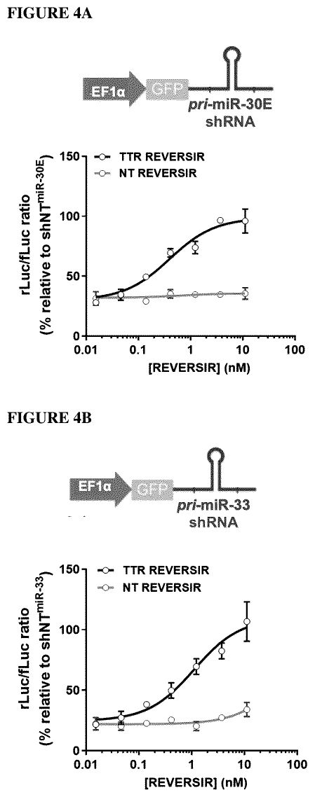

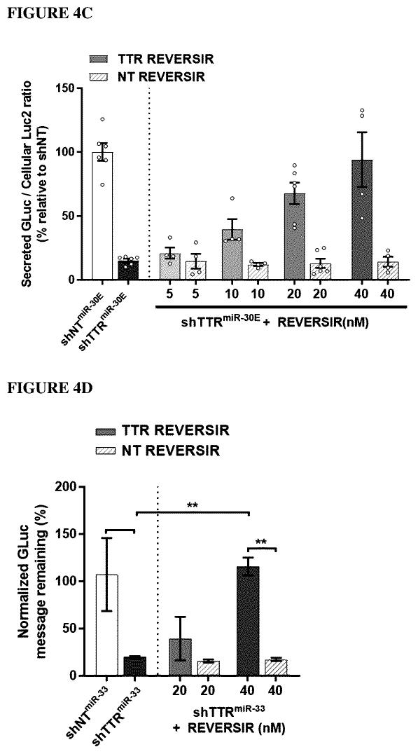

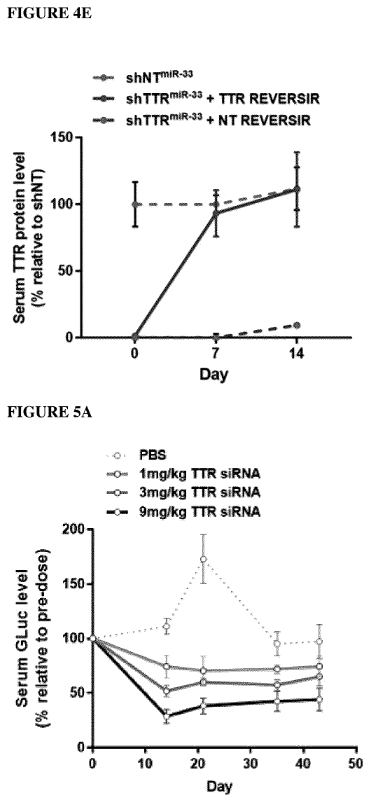

The potential of a single agent approach for AAV transgene control whereby silencing of expression may be achieved by co-expression of an shRNA in cis from the AAV vector, with induction then conferred by exogenous delivery of REVERSIR (FIG. 1A) was investigated first.²¹ To explore this possibility, the ability of REVERSIR to rescue RNAi silencing triggered by two well-characterized miRNA-embedded shRNA scaffolds using luciferase assays in cultured cells was initially examined. Transthyretin (TTR)-targeting shRNA or control non-targeting (NT) shRNA embedded within a miR-30E³² (FIG. 4A) or mouse miR-33³⁰ (FIG. 4B) pri-miRNA scaffold were expressed and assessed in trans repression of a luciferase reporter containing a fully complementary TTR target element within the 3′ UTR. Expression of TTR shRNA from both scaffolds resulted in significant suppression of luciferase reporter activity relative to the NT shRNA. In both cases, dose-dependent recovery of luciferase reporter expression was observed with co-transfection of 22-mer TTR-REVERSIR but not with control non-targeting (NT) REVERSIR of the same length and chemistry.

Next, single self-regulating AAV vector that would concurrently express both a regulatory shRNA along with a transgene mRNA harboring a cognate 3′ UTR target site for the shRNA ws generated. To establish this, the miR-30E- or miR-33-scaffolded TTR shRNA cassette was embedded into a chimeric intron residing between a Gaussia luciferase (GLuc) transgene and liver-specific TBG promoter and a single fully matched binding site for the TTR shRNA was inserted within the 3′ UTR of Gluc (FIG. 1B). This configuration allows expression of the regulatory shRNA and transgene mRNA to be coupled within a single transcriptional unit but permits each to be processed independently following pre-mRNA splicing in the nucleus. It was anticipated that once the shRNA is processed intracellularly, the resulting RISC-loaded antisense strand can mediate binding and cleavage of the AAV-expressed transgene in the cytoplasm, which can then be reversed by exogenous treatment with REVERSIR (FIG. 1A). In vitro evaluation of putative self-silencing AAV plasmids demonstrated robust basal downregulation of the secreted Gluc reporter transgene specifically when both a miRNA-scaffolded TTR shRNA and a TTR shRNA target site were both present in the vector, relative to matched constructs expressing the NT shRNA or NT target site (*p<0.0001 NT vs. TTR shRNA) (FIGS. 1C, 1D, and 4C). As expected given the need for intracellular processing of intronic shRNA into active RNAi effectors, longitudinal monitoring of transgene expression in vitro showed delayed suppression of GLuc transgene expression, Reporter levels were comparable between self-regulating and non-regulating designs at early timepoints with significant silencing evident beginning at 14 h post-transfection (shTTR/NT-ts vs. shTTR/TTRts 14 h, 22 h, 46 h p<0.01) (FIG. 1C). Co-transfection of 22-mer REVERSIR directed against the TTR shRNA resulted in a robust and specific abrogation of shRNA-mediated silencing and recovery of Gluc expression (TTR shRNA+40 nM TTR REVERSIR vs. TTR shRNA+40 nM NT REVERSIR *p<0.01) (FIGS. 1D and 4C). Consistent with blockade of target RNA cleavage through sequestration of antisense-loaded RISC via REVERSIR, rescue of shRNA-induced knockdown of Gluc mRNA levels in the presence of the TTR but not NT REVERSIR was observed (FIG. 4D).

The feasibility of this strategy for regulation of AAV transgene expression in vivo was next assessed with vectors expressing miR-33-embedded shRNA cassettes since prior studies had shown that the pri-miR-33 backbone retained high efficacy without compromising rAAV genome integrity during viral replication.³⁰ Two AAV-Gluc reporter constructs that share a high degree of sequence similarity, both expressing intronically-encoded miR-33-embedded TTR shRNA, and differing only with respect to the presence of either a fully complementary (TTR-ts) or scrambled (NT-ts) target site within the Gluc 3′ UTR were compared. Intravenous administration of the self-silencing TTR shRNA-expressing AAV led to robust knockdown of endogenous TTR protein levels that was completely reversed by exogenous dosing of TTR REVERSIR, establishing successful intron-driven shRNA RNAi and blockade of active RISC with REVERSIR (FIG. 4E). Importantly, in line with the in vitro findings, expression of the self-silencing AAV containing both an intronic miR-33 TTR shRNA and its complementary 3′ UTR binding site (shTTR/TTR-ts) lead to a sustained knockdown (˜76%) of secreted Gluc reporter levels, relative to the non-self-silencing (shTTR/NT-ts) vector design. Serum reporter expression was recovered to levels comparable to the control non-self-silencing vector within 7 days following molar equivalent subcutaneous dosing of a either short 9-mer (5 LNA, 5 PS) or long 22-mer (5 LNA, full PS) TTR REVERSIR, with induction lasting for at least 6 weeks (D49 *p>0.9 shTTR/NT-ts+vehicle vs. shTTR/TTR-ts+9-mer or 22-mer TTR-REVERSIR). No significant Gluc induction was observed upon treatment with equivalent doses of the control NT REVERSIRs [Two-way ANOVA main effect of treatment F (1,160)=66.42 p<0.001; Tukey post-hoc p<0.0001 shTTR/NT-ts+vehicle vs. shTTR/TTR-ts+22-mer or 9-mer NT REVERSIR) (FIG. 1E). As expected, qRT-PCR analyses also revealed rescue of Gluc transcript levels in the presence of TTR-REVERSIR relative to the NT REVERSIR control (FIG. 1F).

While long-term induction may be desirable in certain contexts, the flexibility and utility of an RNAi/REVERSIR switch approach would be greatly enhanced by the ability to modulate the duration of REVERSIR action to achieve more transient upregulation of transgene expression. It was hypothesized that reducing the metabolic stability of the REVERSIR may lead to its more rapid intracellular clearance, thereby facilitating resumption of RISC-mediated target engagement and silencing. Towards this end, mice transduced with self-silencing shTTR/TTR-ts AAV were dosed with an 9-mer TTR REVERSIR designed with lower LNA and PS content (TTR REVERSIR 2 with 3 LNA and 3 PS) to diminish metabolic stability. Both the evaluated doses (0.1 and 0.3 mg/kg) of TTR REVERSIR 2, lead to initial recovery of Gluc transgene expression back to control levels by D6 (*p>0.1 shTTR/NT-ts+vehicle vs. shTTR/TTR-ts+TTR REVERSIR 2). Consistent with reduced metabolic stability and rapid clearance, dose-dependent resumption of Gluc silencing was observed over time, with levels reverting to those of NT REVERSIR-treated mice by D19 for the 0.1 mg/kg (*p>0.3 TTR REVERSIR 2 vs. NT REVERSIR) and D47 for the 0.3 mg/kg (*p>0.9) treatment groups (FIG. 1G). A second peak in Gluc levels was observed following a re-challenge with another dose of TTR REVERSIR 2 on D47, highlighting the potential for repeated induction of transgene expression with tunable REVERSIR designs.

Results from these studies suggest a viable single-agent strategy involving constitutive self-suppression of the AAV transgene that may be counteracted by exogenous administration of REVERSIR to induce expression transiently or for a prolonged period of time.

REVERSIR Blockade of Exogenous siRNA Activity Affords ON Switch and Dosage Control of AAV Transgenes

Extensive chemical modification of siRNAs, along with conjugation to novel ligands, have facilitated marked enhancements in potency, duration of action, and functional delivery to a broader range of extrahepatic tissues.^{11,12,13,17} Current siRNAs featuring enhanced stabilization chemistry (ESC) designs display durable target silencing that can persist for months in preclinical species and humans following a single administration.^{11,12,13} Accordingly, it was postulated that the long-acting and reversible properties of siRNA activity may be uniquely suited to be repurposed as a programmable and user-specified system to achieve exogenous dosage control of AAV transgene expression.

Using hepatotropic AAV8 as a model, it was sought to probe whether exogenous administration of GalNAc-conjugated siRNA and REVERSIR may enable on-demand modulation of AAV-expressed transgenes (FIG. 2A). Mice were injected with an AAV8 vector encoding a bicistronic transcript composed of peripheral myelin protein-22 (PMP-22) and a Gluc reporter, with a single fully complementary binding site for a TTR-targeting siRNA inserted directly following the termination codon within the 3′ UTR (FIG. 2B; left). Subcutaneous (SC) treatment with a single dose of an GalNAc-TTR siRNA resulted in sustained dose-dependent reduction of AAV-driven serum Gluc expression, with the highest dose of 9 mg/kg showing 71% silencing at 14 days and greater than 50% knockdown up to 45 days post-dose. (FIG. 5A). Two weeks following siRNA treatment, mice received a single molar equivalent dose of either full-length (22-mer) or short 9-mer TTR REVERSIR, compared to chemistry- and length-matched NT REVERSIR as controls (FIG. 2B; right). Both the 22-mer and 9-mer TTR REVERSIRs efficiently counteracted silencing activity by the 9 mg/kg siRNA dose, leading to prolonged and comparable induction of Gluc expression to levels similar to that of vehicle-treated controls for at least 4 weeks (D43 *p>0.1 PBS vs. siRNA+9-mer or 22-mer REVERSIR; *p=0.7 22-mer TTR REVERSIR vs. 9-mer REVERSIR) (FIG. 2C). In contrast, Gluc reporter knockdown was unaffected in the presence of NT REVERSIRs, with reporter suppression remaining comparable to mice treated with siRNA alone for the evaluated duration (D42 *p>0.9 siRNA+Vehicle vs. siRNA+22-mer or 9-mer NT REVERSIR). The lower siRNA dose of 3 mg/kg allowed better discrimination of the expected length dependence in REVERSIR activity, with the 9-mer REVERSIR displaying a more robust and earlier recovery of Gluc upregulation as compared to the longer 22-mer design. Downstream qRT-PCR analyses showed that treatment with GalNAc-TTR siRNA resulted in significant reductions in mRNA levels of Gluc (FIG. 2D) and endogenous TTR (FIG. 5C), which were both restored to baseline following administration of 9-mer TTR REVERSIR but not the control NT REVERSIR, indicating blockade of RNAi-mediated transcript degradation by REVERSIR. The titratability of transgene induction was next tested by monitoring Gluc expression following dosing of siRNA-treated mice with increasing amounts of 9-mer TTR REVERSIR. Dose-dependent upregulation of Gluc levels one week post-dose were observed, demonstrating that REVERSIR can allow fine-tuning of AAV transgene expression to the desired level by modulating the magnitude of siRNA-mediated silencing activity (FIG. 2E).

To demonstrate that exogenous dosing of siRNA with REVERIR represents a generalizable approach for AAV transgene regulation, additional AAV vectors in which either the identity of the expressed transcript and/or the regulatory siRNA sequence were varied were evaluated. Mice transduced with an AAV8 vector conferring expression of human Angiopoietin-like 3 (hANGPTL3) with a unique 3′ UTR target site for a Gluc siRNA in order to selectively target the exogenous transgene. Inherent potency of the Gluc siRNA was assessed in vitro using an on-target dual luciferase vector, demonstrating dose-dependent activity repression with an EC₅₀ of 1.9 nM. Co-transfection of Gluc siRNA with its cognate 22-mer REVERSIR yielded concentration-dependent recovery of luciferase expression to baseline levels, whereas the 9-mer Gluc REVERSIR displayed lower potency, recapitulating prior studies showing poorer performance of shorter REVERSIRs when delivered by lipid-based transfection (FIG. 5D). Treatment of AAV-transduced mice with 9 mg/kg Gluc siRNA resulted in 91.7% suppression of serum hANGPTL3 protein concentrations at nadir (D14), with significant knockdown >80% detected up to D35 (D14, D21, D35 *p<0.05 PBS vs. siRNA+Vehicle). Subcutaneous administration of 3 mg/kg 9-mer Gluc REVERSIR, but not NT REVERSIR, reverted serum hANGPTL3 to levels indistinguishable from vehicle controls with protein upregulation lasting for 3 weeks post-induction (D35 *p=0.7 PBS vs. siRNA+Gluc REVERSIR; *p<0.01 PBS vs. siRNA+9-mer NT REVERSIR) (FIGS. 2F and 5E). These data demonstrate that different siRNA regulator sequences may be employed interchangeably in combination with a generalized REVERSIR template to achieve off- and on-state control of AAV transgenes. To address whether transcript secondary structure might impact the efficacy of transgene silencing, an additional AAV construct encoding bicistronic expression of Gluc reporter with a 3′ UTR TTR siRNA target site as described previously was evaluated. However, in this instance, the coding sequence of PMP-22 was replaced with that of a different gene, human Factor XII (hF12). Durable and significant reduction in Gluc levels of >60% were detected up to D49, with maximal knockdown of 73.7% occurring at 2-weeks post-siRNA dose (D14 through 49 *p<=0.05 PBS vs. siRNA+Vehicle). In comparison to treatment with siRNA alone or with a NT REVERSIR, mice that received the TTR REVERSIR displayed significant upregulation of reporter expression up to D70 (D21 through D70 *p<0.01 siRNA alone vs. siRNA+TTR REVERSIR; *p<0.01 TTR REVERSIR vs. NT REVERSIR) (FIGS. 2G and 5F), signifying generalizable translation of this regulatory switch approach regardless of differences in the identity of the virally-expressed transcript. Collectively, these results demonstrate that titratable regulation of liver-directed AAV transgenes may be achieved by exogenous delivery of GalNAc-conjugated siRNAs and REVERSIR.

Identification of Highly Active AAV Transgene Regulator siRNA Sequences with High Specificity

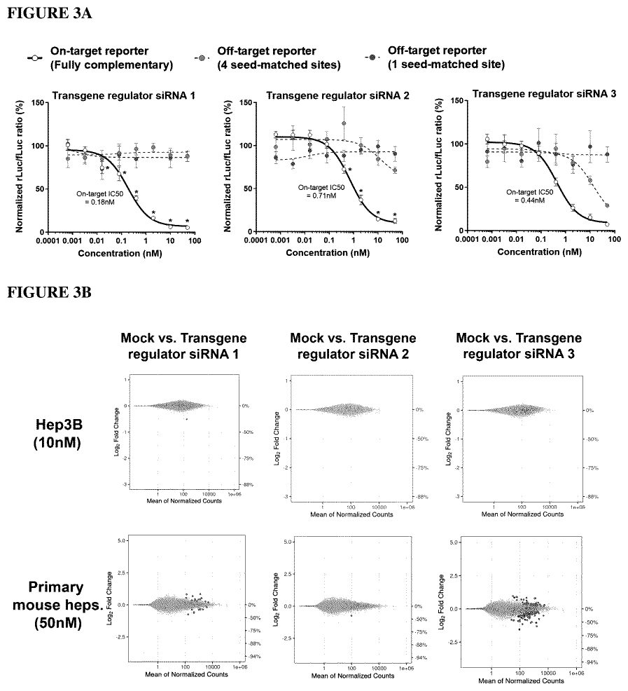

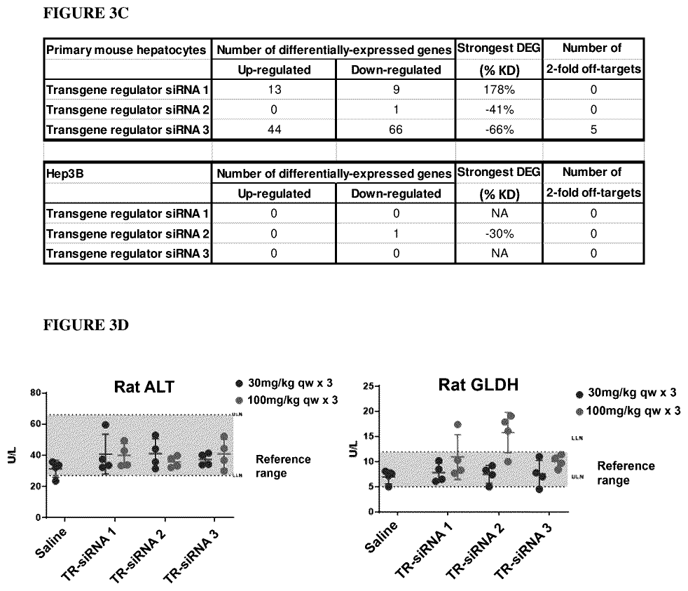

Practical in vivo application of an RNAi-based regulatory switch for AAV vectors necessitates highly selective and unique siRNA sequences that allow RNAi activity to be exclusively directed towards the intended exogenously-expressed transgene, while simultaneously mitigating potential sequence-dependent off-target effects on the endogenous transcriptome. Using bioinformatic approaches, three candidate AAV transgene regulatory siRNAs that were predicted to have favorable thermodynamic properties for RISC loading and RNAi functionality were identified. Importantly, the siRNAs were designed to have little to no sequence complementarity to any annotated genes in human, cynomolgus monkey, rat, and mouse transcriptomes (Tables 2 and 3). To probe their intrinsic silencing efficacy, reporter activity was assessed in vitro in response to increasing doses of the AAV transgene siRNAs in the presence of dual luciferase vectors bearing a single copy of a perfectly complementary target site in the 3′ UTR. All of the siRNAs exhibited significant dose-dependent on-target repression of luciferase reporter activity, with IC50 values in the low nanomolar range [0.18 nM, 0.71 nM, and 0.44 nM for siRNAs 1-3 respectively) (FIG. 3A). To evaluate the potential for microRNA-like off-target knockdown, the siRNAs were co-transfected with luciferase sensors containing either a single copy or four tandem repeats of a site complementary to the seed region of the siRNA antisense strand. In both cases, no significant microRNA-like off-target luciferase reporter repression was observed at doses ranging from 0.64 pM to 50 nM. (FIG. 3A).

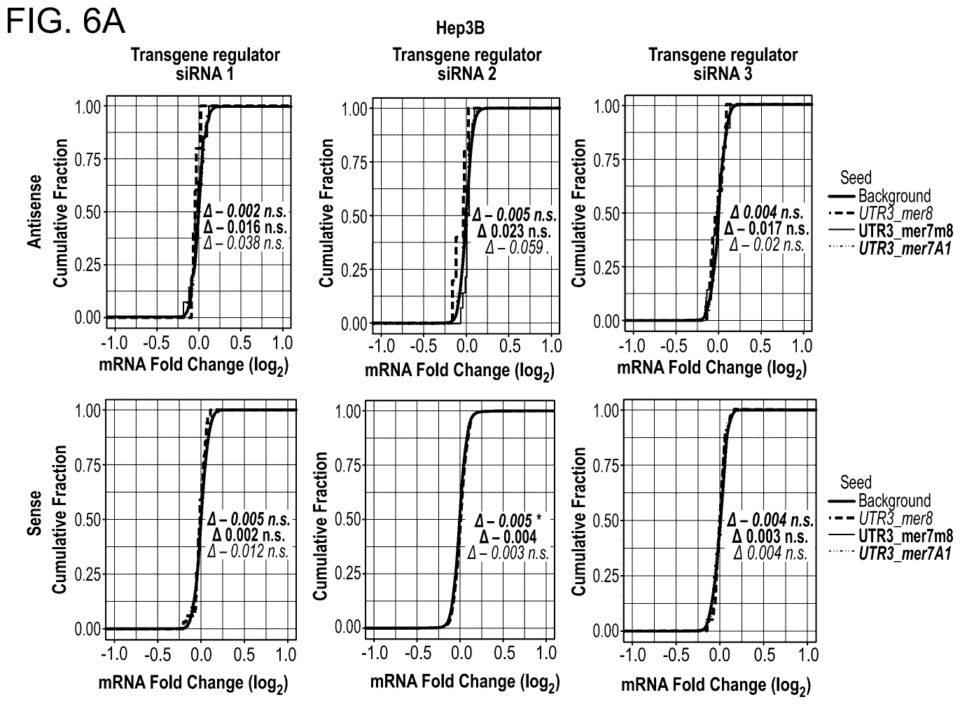

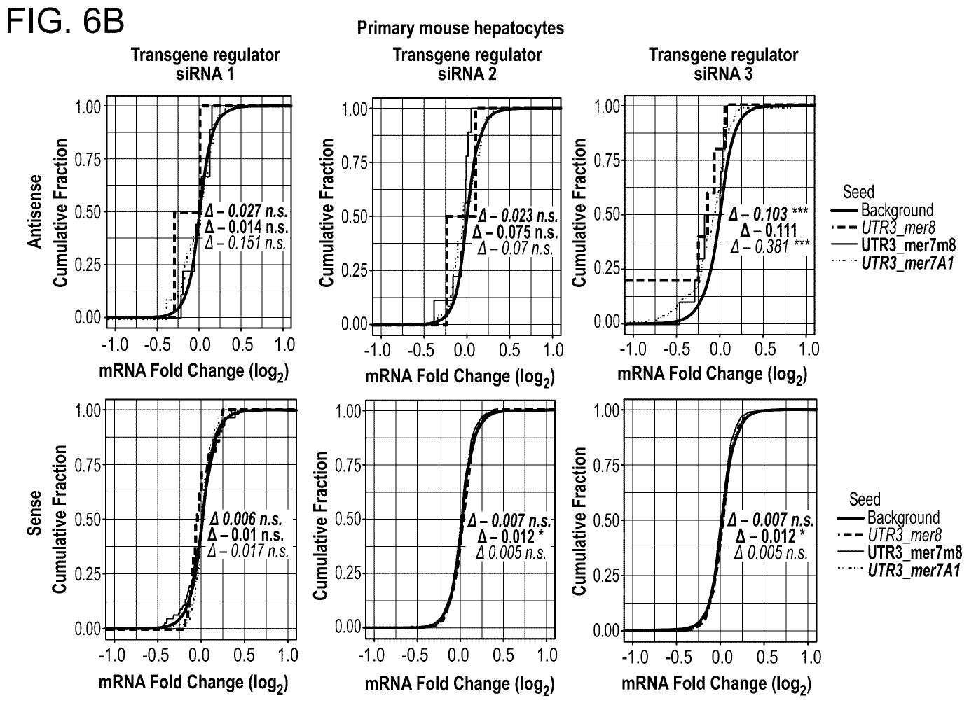

To characterize the potential for sequence-dependent independent off-target effects more broadly, these candidate regulator siRNAs were transfected into Hep3B cells and primary murine hepatocytes to assess their global impact on the human and mouse transcriptomes by RNA-seq. In both cell types, no statistically significant impact on the cumulative expression of mRNA transcripts containing three different types of seed-matches relative to background genes lacking those seed-matches were observed (FIGS. 6A and 6B). This pattern showing lack of dysregulation of transcripts harboring predicted 7mer and 8mer seed matches was also recapitulated when comparing log 2 fold-changes in mRNA expression levels relative to mock control (MA plot in FIG. 3B). All three siRNAs generally displayed minimal off-target signatures, with 2 of the 3 siRNAs showing no transcripts with a magnitude of dysregulation exceeding 2-fold. Additionally, it was observed that there was no significant pattern of enrichment for the seed region of the sense strand, demonstrating a lack of productive RISC loading and activity of the sense strand with these sequences and chemical modification template (FIGS. 6A and 6B).

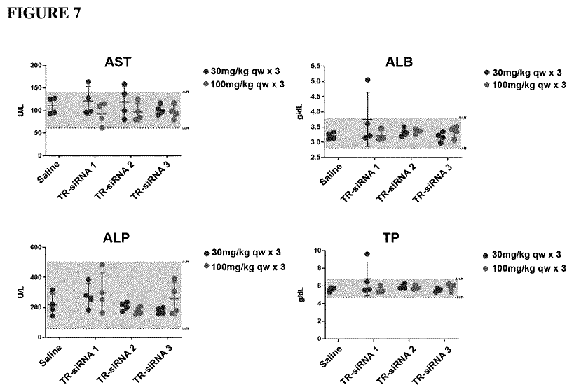

To address whether the observed lack of off-target gene dysregulation is sufficient to mitigate hepatotoxicity in vivo, the three transgene regulator siRNAs were administered at toxicological doses in rats. Rats received once weekly subcutaneous injections (qw×3 doses) of 30 or 100 mg/kg siRNA, representing 2-3 log exaggeration of the pharmacological dose range. All three siRNAs did not show any significant liver enzyme elevations, except for a mild increase in GLDH with siRNA 2 (FIGS. 3C and 7). Minimal to no microscopic liver findings were observed, including fibrosis, single cell necrosis, and hepatocellular degeneration.

Overall results from the in vitro RNA-seq and rat toxicity screening efforts establish these siRNAs as potent RNAi triggers with high on-target specificity. These AAV transgene siRNA sequences display minimal propensity for off-target gene disruption and in vivo toxicity, demonstrating their use as regulators of exogenous transgenes with favorable safety profiles.

### Discussion

A lack of broadly applicable methods to control the dosage of therapeutic transgenes is a central challenge surrounding AAV gene therapies, since most currently rely on persistent constitutive expression following a single administration. RNA-based molecular switches are emerging as attractive candidates to regulate transgene expression from AAVs owing to their small size, sequence specificity, and reduced potential for immunogenicity.⁵ Recent developments in riboswitches based on steric oligo blockade of self-cleaving ribozyme activity and small molecule regulation of alternative RNA splicing have shown promise in preclinical models but their applicability in clinical settings is unknown.³⁵⁻³⁶ Described herein is a generalizable and clinically viable approach for dosage control of AAV-delivered cargos involving dynamic, robust, and reversible control via RNAi with benefit of infrequent dosing. Reversal of RNAi-mediated transgene knockdown with REVERSIR in both a dual agent setting involving exogenous administration of chemically modified siRNA, and a single agent setting where co-delivery of vectorized shRNA enables constitutive silencing is demonstrated herein. Both approaches provide durable dampening of transgene expression that may be abrogated with REVERSIR to yield weeks- to months-long elevation in protein levels in mice following a single dose of the inducer.

Unlike riboswitch designs that depend on self-cleaving ribozyme activity or splicing-dependent transgene cassettes, both of which afford extremely tight control in the off-state, an RNAi mechanism is unlikely to completely abolish basal transgene expression. Nevertheless, clinical trials evaluating investigational RNAi therapeutics have shown robust and prolonged target knockdown; results from the ORION phase 3 study of a GalNAc-siRNA targeting PCSK9 support a once-every-6-month dosing regimen. Dose-dependent lowering of AAV-expressed protein levels that were maintained for over a month in rodents after a single dose of siRNA, or were constitutively suppressed in the case of vectorized shRNA have been demonstrated herein.

Despite the slower kinetics of onset and inability to wholly inactivate transgene expression, there are certain settings where the durability of RNAi activity and the potential for infrequent dosing have distinct utility for the regulation of AAV gene therapies. For instance, RNAi is well suited for regulation of certain cargoes, such as monoclonal antibodies, where dampening antibody concentrations below the required threshold for therapeutic effect may be sufficient to minimize adverse effects.⁴⁵ In addition, using RNAi to address the challenge of dose scaling and management for highly active transgenes, such as the Padua variant of FIX, where small differences in vector dose lead to exaggerated changes in protein production is useful.⁴⁶ Systemic administration can also produce variable levels of transgene expression in response to the same vector dose, consequently increasing the risk of adverse toxicity if protein expression far exceeds the therapeutic range. This was recently highlighted in trials evaluating AAV-FVIII for the treatment of hemophilia A where a high degree of variation in FVIII activity levels was observed among participants.^{41,42} One could use RNAi drugs to titrate transgene expression to within the therapeutic window and thereby mitigate potential negative consequences associated with over-production of the therapeutic protein, such as increased thrombotic risk in the case of high FVIII levels. In addition to initial dose titration after AAV dosing, RNAi approaches enable transgene expression to be dynamically modified as the disease state evolves. Incorporation of an RNAi-based safety switch could allow temporary cessation of treatment if needed. With vectorized shRNA co-expression, the potential to delay stable transgene expression until after immune responses subside represents another potential key benefit.⁴⁷

A key piece of data that emerged from the studies described herein was that REVERSIR treatment afforded complete recovery of the full range of transgene expression that was observed in the absence of the RNAi regulatory module. A previously reported RNAi on-switch design based on ligand-promoted occlusion of a microRNA target site with a competing complementary strand yielded a maximal recovery of ˜20% of non-regulated expression, likely owing to the remarkable efficiency with which RISC recognizes and binds its targets.⁹ The findings described herein deomonstrate that blocking RNAi activity directly at the level of catalytic RISC provides a significantly more efficient and robust means of regulating the on-state.

Whereas this approach facilitated maximal recovery of transgene expression, the overall dynamic range for induction was still relatively modest, with ˜5-10 fold regulation of reporter gene expression detected in vivo. However, in line with previous studies showing that more efficient RNAi-mediated silencing results in wider dynamic ranges for induction⁹, the most significant fold upregulation with AAV-expressed hANGPTL3 was observed where >90% suppression of basal transgene expression with siRNA was able to be achieved. On the other hand, on-state control via regulation of alternative splicing with small molecules or ribozyme activity, with steric oligos achieve a much larger magnitude (>100-fold) but a relatively transient duration of transgene induction. As a result, chronic administration of the inducer might be required to maintain persistent expression of the therapeutic transgene, particularly for proteins and peptides with a short half-life. In line with the durability of RNAi responses, it was shown that a single dose of REVERSIR produced weeks-long elevations in GLuc reporter, which has an extremely short serum half-life of 20 minutes. It was also demonstrated that duration of induction can be tuned by modifying the chemical stability of the REVERSIR, thereby adding a layer of versatility for different gene therapy applications. Additional refinements to the metabolic stability and dose of the REVERSIR would shorten timescales of induction even further.

Also described herein are potent RNAi-active siRNA sequences that can be incorporated into episomal vectors to selectively regulate exogenous transgene expression. Several studies have shown that hybridization of the siRNA seed region to complementary sequences within the 3′ UTR of mature transcripts is the principal driver of RNAi-mediated off-target gene dysregulation and in vivo toxicity. Therefore, siRNAs with seed-matched target sequences that occur at low frequency and whose full-length sequences lack homology to expressed transcripts across mouse, rat, cynomolgous monkey, and human genomes were prioritized. Minimal to no seed- or non-seed-mediated dysregulation of endogenous mRNA expression in both human and mouse hepatic cell lines at high doses were observed. While these siRNAs were not explicitly evaluated in rat and cynomolgous monkey cell lines, they should be equivalently quiescent since they were found by brute force prediction to lack homology to any genomically-expressed transcripts across all tissues. This specificity feature across preclinical species and humans, increases the potential for translational into the clinic with favorable safety profile. Although these siRNAs were only evaluated in hepatocytes, that they should be equivalently quiescent in cell types from other tissues. All three AAV transgene siRNAs demonstrated minimal clinical and histopathological findings at chronic toxicological doses in rodents. These data demonstrate that these siRNAs may be administered at relatively high doses if needed with minimal risk of off-target effects. Overall, these features increase their clinical translation with favorable safety profile. The studies described herein utilized a regulatory element consisting of a single fully-matched binding site within the 3′ UTR of the AAV transgene. The sensitivity of the RNAi-driven regulation can be further influenced by modifying the local sequence around the target site, altering the proximity of the target site to the transgene stop codon, or by incorporating sites for multiple siRNAs.

While the current studies are limited to control of hepatocyte-directed transgene expression with GalNAc-conjugated siRNAs and REVERSIRs, novel delivery solutions such as C16 will broaden the scope of RNAi-based strategies for control of AAV vectors targeting a wide range of tissues.¹⁷ For extrahepatic applications, constitutive basal transgene silencing via vectorized RNAi delivery might be preferable since it obviates the need for exogenous siRNA and enables a single agent strategy involving dosing of REVERSIR alone.

In summary, the present invention provides simple and clinically adaptable tools and methods for regulated protein expression from vectors, e.g., viral, e.g., AAV, vectors, with a unique profile and use case compared to other transgene regulatory modalities.

TABLE 2
Unmodified Nucleotide Sequences of Universal dsRNA Agents

| | Sense Sequence | SEQ | Antisense Sequence | SEQ |
|---|---|---|---|---|
| Duplex ID | 5′ to 3′ | ID NO: | 5′ to 3′ | ID NO: |
| AD-1548706 | AUAGACGUGAUUAAUCGCGUU | 5 | AACGCGAUUAAUCACGUCUAUCU | 27 |
| AD-1548707 | AAGAUGAUCCUUAUCGCGAAU | 6 | AUUCGCGAUAAGGAUCAUCUUUA | 28 |
| AD-1548708 | CCGCGGAUUAUUUAUCGCGUU | 7 | AACGCGAUAAAUAAUCCGCGGAU | 29 |
| AD-1548709 | AAAGUGUCGAAUUUUCGCGAU | 8 | AUCGCGAAAAUUCGACACUUUUU | 30 |
| AD-1548710 | CACCUCCUAAUUAAUCGCGUU | 9 | AACGCGAUUAAUUAGGAGGUGGU | 31 |
| AD-1548711 | AAAGCUAGGUUUAUACGACGU | 10 | ACGUCGUAUAAACCUAGCUUUCA | 32 |
| AD-1548712 | GAGGACUCAAUUUAUCGCGUU | 11 | AACGCGAUAAAUUGAGUCCUCCA | 33 |
| AD-1548713 | AAAGACGUACUUUAAUCGCGU | 12 | ACGCGAUUAAAGUACGUCUUUAU | 34 |
| AD-1548714 | UCAGUUGACCAUAUCGUACGU | 13 | ACGUACGAUAUGGUCAACUGAAA | 35 |
| AD-1548715 | UCUGUAGUGGAUAUACGACGU | 14 | ACGUCGUAUAUCCACUACAGAUU | 36 |
| AD-1548716 | UAGUACGACUAUUUAUCGCGU | 15 | ACGCGAUAAAUAGUCGUACUAGU | 37 |
| AD-1548717 | AAAGUCGUCUUUACGUAUCGU | 16 | ACGAUACGUAAAGACGACUUUUU | 38 |
| AD-1548718 | AACGUACACACUAAUACGCGU | 17 | ACGCGUAUUAGUGUGUACGUUUU | 39 |
| AD-1548719 | AACCACAUCCUUAUACGACGU | 18 | ACGUCGUAUAAGGAUGUGGUUAA | 40 |
| AD-1548720 | AUGAACGACUUUUUAUCGCGU | 19 | ACGCGAUAAAAAGUCGUUCAUGA | 41 |
| AD-1548721 | UUUCAUGCGAUUACGUAUCGU | 20 | ACGAUACGUAAUCGCAUGAAAAA | 42 |
| AD-1548722 | AAACGAGUGUUUUAUCGCGAU | 21 | AUCGCGAUAAAACACUCGUUUAA | 43 |
| AD-1548723 | AUCGUCUAGUAUUUUCGCGAU | 22 | AUCGCGAAAAUACUAGACGAUCA | 44 |
| AD-1548724 | UUUCAUAGCGUUACGUACGUU | 23 | AACGUACGUAACGCUAUGAAAUU | 45 |
| AD-1548725 | ACCCAGAUAUUUUUAUCGCGU | 24 | ACGCGAUAAAAAUAUCUGGGUCG | 46 |
| AD-1548726 | UAUCGUCUGAAUACGUACGUU | 25 | AACGUACGUAUUCAGACGAUAGC | 47 |
| AD-1548727 | UUACAUCCGUUUUUUCGCGAU | 26 | AUCGCGAAAAAACGGAUGUAACG | 48 |

TABLE 3
Modified Nucleotide Sequences of Universal dsRNA Agents

| Duplex | | SEQ ID | | SEQ | | SEQ |
|---|---|---|---|---|---|---|
| ID | Sense Sequence 5′ to 3′ | NO: | Antisense Sequence 5′ to 3′ | ID NO: | Target Sequence 5′ to 3′ | ID NO: |
| AD- | asusagacGfuGfAfUfuaaucgcgu | 49 | asAfscgcGfaUfUfaauc AfcGfucuaus | 71 | AGAUAGACGUGAUUAAUCGCGUU | 93 |
| 1548706 | uL96 | | csu | | | |
| AD- | asasgaugAfuCfCfUfuaucgcgaa | 50 | asUfsucgCfgAfUfaaggAfuCfaucuu | 72 | UAAAGAUGAUCCUUAUCGCGAAU | 94 |
| 1548707 | uL96 | | susa | | | |
| AD- | cscsgcggAfuUfAfUfuuaucgcgu | 51 | asAfscgcGfaUfAfaauaAfuCfcgcggs | 73 | AUCCGCGGAUUAUUUAUCGCGUU | 95 |
| 1548708 | uL96 | | asu | | | |
| AD- | asasagugUfcGfAfAfuuuucgcga | 52 | asUfscgcGfaAfAfauucGfaCfacuuus | 74 | AAAAAGUGUCGAAUUUUCGCGAU | 96 |
| 1548709 | uL96 | | usu | | | |
| AD- | csasccucCfuAfAfUfuaaucgcgu | 53 | asAfscgcGfaUfUfaauuAfgGfaggug | 75 | ACCACCUCCUAAUUAAUCGCGUU | 97 |
| 1548710 | uL96 | | sgsu | | | |
| AD- | asasagcuAfgGfUfUfuauacgacg | 54 | asCfsgucGfuAfUfaaacCfuAfgcuuus | 76 | UGAAAGCUAGGUUUAUACGACGU | 98 |
| 1548711 | uL96 | | csa | | | |
| AD- | gsasggacUfcAfAfUfuuaucgcgu | 55 | asAfscgcGfaUfAfaauuGfaGfuccucs | 77 | AUAAAGACGUACUUUAAUCGCGU | 99 |
| 1548712 | uL96 | | csa | | | |
| AD- | asasagacGfuAfCfUfuuaaucgcg | 56 | asCfsgcgAfuUfAfaaguAfcGfucuuu | 78 | AUAAAGACGUACUUUAAUCGCGU | 100 |
| 1548713 | uL96 | | sasu | | | |
| AD- | uscsaguuGfaCfCfAfuaucguacg | 57 | asCfsguaCfgAfUfauggUfcAfacugas | 79 | UUUCAGUUGACCAUAUCGUACGU | 101 |
| 1548714 | uL96 | | asa | | | |
| AD | uscsuguaGfuGfGfAfuauacgacg | 58 | asCfsgucGfuAfUfauccAfcUfacagas | 80 | AAUCUGUAGUGGAUAUACGACGU | 102 |
| 1548715 | uL96 | | usu | | | |
| AD- | usasguacGfaCfUfAfuuuaucgcg | 59 | asCfsgcgAfuAfAfauagUfcGfuacuas | 81 | ACUAGUACGACUAUUUAUCGCGU | 103 |
| 1548716 | uL96 | | gsu | | | |
| AD- | asasagucGfuCfUfUfuacguaucg | 60 | asCfsgauAfcGfUfaaagAfcGfacuuus | 82 | AAAAAGUCGUCUUUACGUAUCGU | 104 |
| 1548717 | uL96 | | usu | | | |
| AD- | asascguaCfaCfAfCfuaauacgcgu | 61 | asCfsgcgUfaUfUfagugUfgUfacguu | 83 | AAAACGUACACACUAAUACGCGU | 105 |
| 1548718 | L96 | | susu | | | |
| AD- | asasccacAfuCfCfUfuauacgacgu | 62 | asCfsgucGfuAfUfaaggAfuGfugguu | 84 | UUAACCACAUCCUUAUACGACGU | 106 |
| 1548719 | L96 | | sasa | | | |
| AD- | asusgaacGfaCfUfUfuuuaucgcg | 63 | asCfsgcgAfuAfAfaaagUfcGfuucaus | 85 | UCAUGAACGACUUUUUAUCGCGU | 107 |
| 1548720 | uL96 | | gsa | | | |
| AD- | ususucauGfcGfAfUfuacguaucg | 64 | asCfsgauAfcGfUfaaucGfcAfugaaas | 86 | UUUUUCAUGCGAUUACGUAUCGU | 108 |
| 1548721 | uL96 | | asa | | | |
| AD- | asasacgaGfuGfUfUfuuaucgcga | 65 | asUfscgcGfaUfAfaaacAfcUfcguuus | 87 | UUAAACGAGUGUUUUAUCGCGAU | 109 |
| 1548722 | uL96 | | asa | | | |
| AD- | asuscgucUfaGfUfAfuuuucgcga | 66 | asUfscgcGfaAfAfauacUfaGfacgaus | 88 | UGAUCGUCUAGUAUUUUCGCGAU | 110 |
| 1548723 | uL96 | | csa | | | |
| AD- | ususucauAfgCfGfUfuacguacgu | 67 | asAfscguAfcGfUfaacgCfuAfugaaas | 89 | AAUUUCAUAGCGUUACGUACGUU | 111 |
| 1548724 | uL96 | | usu | | | |
| AD- | ascsccagAfuAfUfUfuuuaucgcg | 68 | asCfsgcgAfuAfAfaaauAfuCfugggu | 90 | CGACCCAGAUAUUUUUAUCGCGU | 112 |
| 1548725 | uL96 | | scsg | | | |
| AD- | usasucguCfuGfAfAfuacguacgu | 69 | asAfscguAfcGfUfauucAfgAfcgaua | 91 | GCUAUCGUCUGAAUACGUACGUU | 113 |
| 1548726 | uL96 | | sgsc | | | |
| AD- | ususacauCfcGfUfUfuuuucgcga | 70 | asUfscgcGfaAfAfaaacGfgAfuguaas | 92 | CGUUACAUCCGUUUUUUCGCGAU | 114 |
| 1548727 | uL96 | | csg | | | |

TABLE 4
REVERSIR COMPOUNDS

| Universal | | | | | | |
|---|---|---|---|---|---|---|
| siRNA | Reversir | Reversir | | SEQ | | SEQ |
| Target | ID | length | REVERSIR Sequence 5′ to 3′ | ID NO: | Target Sequence 5′ to 3′ | ID NO: |
| AD- | A- | 22-mer | ascscscsasgsasusasusususus(Tlns)(Alns)(Tlns)(m5Clns)gs | 115 | ACCCAGAUAUUUUTATCGCGA | 127 |
| 1548725 | 3303065 | | (m5Clns)gdAL96 | | | |
| | A- | 9-mer | usus(Tln)as(Tln)(m5Cln)gs(m5Clns)(Gln)dAL96 | 116 | UUTATCGCGA | 128 |
| | 3303067 | | | | | |
| AD | A- | 22-mer | usasgsusascsgsascsusasusus(Tlns)(Alns)(Tlns)(m5Clns)gs | 117 | UAGUACGACUAUUTATCGCGA | 129 |
| 1548716 | 3303066 | | (m5Clns)gdAL96 | | | |
| | A- | 9-mer | usus(Tln)as(Tln)(m5Cln)gs(m5Clns)(Gln)dAL96 | 118 | UUTATCGCGA | 130 |
| | 3303067 | | | | | |
| AD- | A- | 22-mer | asasasgsuscsgsuscsusususas(m5Clns)(Glns)(Tlns)(Alns)us | 119 | AAAGUCGUCUUUACGTAUCGA | 131 |
| 1548717 | 3302871 | | (m5Clns)gdAL96 | | | |
| | A- | 9-mer | usas(m5Cln)gs(Tln)(Aln)us(m5Clns)(Gln)dAL96 | 120 | UACGTAUCGA | 132 |
| | 3302875 | | | | | |
| AD- | A- | 22-mer | asasasgsusgsuscsgsasasusus(Tlns)(Tlns)(m5Clns)(Glns)cs | 121 | AAAGUGUCGAAUUTTCGCGAA | 133 |
| 1548709 | 3302870 | | (Glns)adAL96 | | | |
| | A- | 9-mer | usus(Tln)us(m5Cln)(Gln)cs(Glns)(Aln)dAL96 | 122 | UUTUCGCGAA | 134 |
| | 3302874 | | | | | |
| AD- | A- | 22-mer | asasascsgsasgsusgsusususus(Alns)(Tlns)(m5Clns)(Glns)cs | 123 | AAACGAGUGUUUUATCGCGAA | 135 |
| 1548722 | 3302869 | | (Glns)adAL96 | | | |
| | A- | 9-mer | usus(Aln)us(m5Cln)(Gln)cs(Glns)(Aln)dAL96 | 124 | UUAUCGCGAA | 136 |
| | 3302873 | | | | | |
| AD- | A- | 22-mer | asasgsasusgsasuscscsususas(Tlns)(m5Clns)(Glns)(m5Clns) | 125 | AAGAUGAUCCUUATCGCGAAA | 137 |
| 1548707 | 3302868 | | gs(Alns)adAL96 | | | |
| | A- | 9-mer | usas(Tln)cs(Gln)(m5Cln)gs(Alns)(Aln)dAL96 | 126 | UATCGCGAAA | 138 |
| | 3302872 | | | | | |

TABLE 5
In vitro luciferase On-Target Potency of Universal dsRNA Agents

| | PBS | | | 0.01 nM | | | 0.1 nM | | | 1n M | | | 10 nM | | |
|---|---|---|---|---|---|---|---|---|---|---|---|---|---|---|---|
| | Mean | | | Mean | | | Mean | | | Mean | | | Mean | | |
| | (%) | SEM | N | (%) | SEM | N | (%) | SEM | N | (%) | SEM | N | (%) | SEM | N |
| AD- | 100 | 9.843496 | 3 | 105.3107 | 13.56149 | 3 | 107.6536 | 1.270329 | 3 | 79.37433 | 6.326838 | 3 | 34.57365 | 1.562546 | 3 |
| 1548706 | | | | | | | | | | | | | | | |
| AD- | 99.99999 | 9.060886 | 3 | 89.55655 | 7.164091 | 3 | 45.60468 | 1.649627 | 3 | 11.02605 | 1.774681 | 3 | 4.345417 | 0.465096 | 3 |
| 1548707 | | | | | | | | | | | | | | | |
| AD- | 100 | 2.721108 | 3 | 104.5333 | 2.866741 | 3 | 95.9001 | 9.655108 | 3 | 69.55306 | 2.20016 | 3 | 25.69401 | 2.249874 | 3 |
| 1548708 | | | | | | | | | | | | | | | |
| AD- | 100 | 3.096701 | 3 | 100.988 | 7.099692 | 3 | 85.90476 | 2.58524 | 3 | 31.10067 | 0.740418 | 3 | 8.266975 | 0.7767 | 3 |
| 1548709 | | | | | | | | | | | | | | | |
| AD- | 99.99999 | 3.512108 | 3 | 97.21867 | 7.290736 | 3 | 106.2903 | 7.863011 | 3 | 70.54031 | 0.662297 | 3 | 32.38354 | 1.741181 | 3 |
| 1548710 | | | | | | | | | | | | | | | |
| AD- | 99.99999 | 5.087655 | 3 | 100.2708 | 7.25597 | 3 | 90.30495 | 3.895551 | 3 | 67.8454 | 7.978696 | 3 | 30.59787 | 2.537093 | 3 |
| 1548711 | | | | | | | | | | | | | | | |
| AD- | 99.99997 | 13.40851 | 3 | 94.80889 | 8.030163 | 3 | 82.03751 | 8.814243 | 3 | 58.40267 | 9.984083 | 3 | 18.84491 | 1.141272 | 3 |
| 1548712 | | | | | | | | | | | | | | | |
| AD- | 100 | 7.475834 | 3 | 101.2088 | 3.561816 | 3 | 87.91335 | 3.570147 | 3 | 41.4004 | 2.276317 | 3 | 13.60588 | 0.993846 | 3 |
| 1548713 | | | | | | | | | | | | | | | |
| AD- | 100 | 2.978287 | 6 | 101.0797 | 6.00358 | 6 | 79.6063 | 9.067951 | 6 | 34.9738 | 3.999246 | 6 | 8.393015 | 1.091314 | 6 |
| 1548714 | | | | | | | | | | | | | | | |
| AD- | 100 | 7.502555 | 3 | 91.97091 | 7.111895 | 3 | 108.6321 | 2.040598 | 3 | 42.88825 | 2.210281 | 3 | 11.85263 | 0.296137 | 3 |
| 1548715 | | | | | | | | | | | | | | | |
| AD- | 99.99999 | 5.863686 | 6 | 96.48905 | 4.160939 | 6 | 80.00647 | 3.916792 | 6 | 24.18157 | 1.858107 | 6 | 7.163752 | 0.780428 | 6 |
| 1548716 | | | | | | | | | | | | | | | |
| AD- | 99.99999 | 4.513342 | 6 | 95.67247 | 4.536762 | 6 | 66.91318 | 3.984807 | 6 | 24.56877 | 1.707934 | 6 | 6.744397 | 0.5392 | 6 |
| 1548717 | | | | | | | | | | | | | | | |
| AD- | 100 | 1.660064 | 6 | 101.9741 | 5.385832 | 6 | 79.15002 | 6.196991 | 6 | 35.61124 | 1.341634 | 6 | 10.7625 | 0.372471 | 6 |
| 1548718 | | | | | | | | | | | | | | | |
| AD- | 100 | 3.057511 | 6 | 96.37554 | 5.301714 | 6 | 87.4103 | 7.438278 | 6 | 38.74572 | 1.970266 | 6 | 15.48581 | 0.833496 | 6 |
| 1548719 | | | | | | | | | | | | | | | |
| AD- | 100 | 4.485297 | 6 | 96.98801 | 4.123085 | 6 | 74.92569 | 3.992402 | 6 | 35.8948 | 4.424529 | 6 | 8.682744 | 0.414754 | 6 |
| 1548720 | | | | | | | | | | | | | | | |
| AD- | 100 | 5.250698 | 6 | 96.61326 | 5.778644 | 6 | 87.04863 | 7.329776 | 6 | 61.06328 | 3.477148 | 6 | 21.47706 | 1.410426 | 6 |
| 1548721 | | | | | | | | | | | | | | | |
| AD- | 100 | 5.151962 | 6 | 84.67402 | 3.425919 | 6 | 64.52089 | 6.671201 | 6 | 17.65779 | 1.610669 | 6 | 8.824126 | 0.814364 | 6 |
| 1548722 | | | | | | | | | | | | | | | |
| AD- | 100 | 4.618495 | 6 | 95.67697 | 4.953171 | 6 | 65.74278 | 4.589406 | 6 | 20.62154 | 1.741484 | 6 | 9.065015 | 0.741591 | 6 |
| 1548723 | | | | | | | | | | | | | | | |
| AD- | 99.99998 | 2.722317 | 6 | 95.80061 | 5.63018 | 6 | 81.93541 | 5.775046 | 6 | 37.79811 | 1.765312 | 6 | 11.31583 | 0.875164 | 6 |
| 1548724 | | | | | | | | | | | | | | | |
| AD- | 100 | 2.19727 | 6 | 95.80767 | 2.386613 | 6 | 77.44083 | 2.617791 | 6 | 25.98591 | 0.455628 | 6 | 10.8214 | 1.47507 | 6 |
| 1548725 | | | | | | | | | | | | | | | |
| AD- | 100 | 1.185009 | 6 | 101.5587 | 2.491656 | 6 | 88.26374 | 3.130755 | 6 | 45.6915 | 1.687426 | 6 | 14.62675 | 0.80635 | 6 |
| 1548726 | | | | | | | | | | | | | | | |
| AD- | 100 | 2.237609 | 6 | 98.67695 | 4.20312 | 6 | 70.2281 | 1.725584 | 6 | 34.08571 | 3.284418 | 6 | 16.81069 | 1.348935 | 6 |
| 1548727 | | | | | | | | | | | | | | | |

### REFERENCES

1. Wang, Dan, Phillip W L Tai, and Guangping Gao. “Adeno-associated virus vector as a platform for gene therapy delivery.” Nature reviews Drug discovery 18.5 (2019): 358-378.
2. Domenger, C., & Grimm, D. (2019). Next-generation AAV vectors—do not judge a virus (only) by its cover. Human molecular genetics, 28 (R¹), R3-R14.
3. Agarwal, S. (2020). High-dose AAV gene therapy deaths. Nat. Biotechnol, 38, 910.
4. Carthew, R. W. & Sontheimer, E. J. Origins and mechanisms of miRNAs and siRNAs. Cell 136, 642-655 (2009).
5. Tickner, Z. J., & Farzan, M. (2021). Riboswitches for Controlled Expression of Therapeutic Transgenes Delivered by Adeno-Associated Viral Vectors. Pharmaceuticals, 14 (6), 554.
6. Kumar, D.; An, C.-I.; Yokobayashi, Y. Conditional RNA Interference Mediated by Allosteric Ribozyme. J. Am. Chem. Soc. 2009,131, 13906-13907.
7. Wong, R. S.; Chen, Y. Y.; Smolke, C. D. Regulation of T cell proliferation with drug-responsive microRNA switches. Nucleic Acids Res. 2018, 46, 1541-1552.
8. Pollak, N. M.; Cooper-White, J. J.; Macdonald, J. Translational control of enzyme scavenger expression with toxin-induced micro RNA switches. Sci. Rep. 2021, 11, 1-12.
9. Mou, H.; Zhong, G.; Gardner, M. R.; Wang, H., Wang, Y.-W.; Cheng, D.; Farzan, M.

Conditional Regulation of Gene Expression by Ligand-Induced Occlusion of a MicroRNA Target Sequence. Mol. Ther. 2018, 26, 1277-1286.
10. Borel, F., Kay, M. A., & Mueller, C. (2014). Recombinant AAV as a platform for translating the therapeutic potential of RNA interference. Molecular Therapy, 22 (4), 692-701.
11. Nair, J. K., Attarwala, H., Sehgal, A., Wang, Q., Aluri, K., Zhang, X., . . . & Maier, M. A. (2017). Impact of enhanced metabolic stability on pharmacokinetics and pharmacodynamics of GalNAc-siRNA conjugates. Nucleic acids research.
12. Foster, D. J., Brown, C. R., Shaikh, S., Trapp, C., Schlegel, M. K., Qian, K., . . . & Milstein, S. (2018). Advanced siRNA designs further improve in vivo performance of GalNAc-siRNA conjugates. Molecular Therapy, 26 (3), 708-717.
13. Brown, C. R., Gupta, S., Qin, J., Racie, T., He, G., Lentini, S., . . . & Jadhav, V. (2020). Investigating the pharmacodynamic durability of GalNAc-siRNA conjugates. Nucleic acids research, 48 (21), 11827-11844.
14. S. F. Garrelfs, Y. Frishberg, S. A. Hulton, M. J. Koren, W. D. O'Riordan, P. Cochat, G. Deschênes, H. Shasha-Lavsky, J. M. Saland, W. G. van't Hoff, D. G. Fuster, D. Magen, S. H. Moochhala, G. Schalk, E. Simkova, J. W. Groothoff, D. J. Sas, K. A. Meliambro, J. Lu, M. T. Sweetser, P. P. Garg, A. K. Vaishnaw, J. M. Gansner, T. L. McGregor, J. C. Lieske, Lumasiran, an RNAi Therapeutic for Primary Hyperoxaluria Type 1. N. Engl. J. Med. 384, 1216-1226 (2021).
15. M. Balwani, E. Sardh, P. Ventura, P. A. Peiró, D. C. Rees, U. Stölzel, D. M. Bissell, H. L. Bonkovsky, J. Windyga, K. E. Anderson, C. Parker, S. M. Silver, S. B. Keel, J.-D. Wang, P. E. Stein, P. Harper, D. Vassiliou, B. Wang, J. Phillips, A. Ivanova, J. G. Langendonk, R. Kauppinen, E. Minder, Y. Horie, C. Penz, J. Chen, S. Liu, J. J. Ko, M. T. Sweetser, P. Garg, A. Vaishnaw, J. B. Kim, A. R. Simon, L. Gouya, Phase 3 Trial of RNAi Therapeutic Givosiran for Acute Intermittent Porphyria. N. Engl. J. Med. 382, 2289-2301 (2020).
16. D. Adams, A. Gonzalez-Duarte, W. D. O'Riordan, C.-C. Yang, M. Ueda, A. V. Kristen, I. Tournev, H. H. Schmidt, T. Coelho, J. L. Berk, K.-P. Lin, G. Vita, S. Attarian, V. Planté-Bordeneuve, M. M. Mezei, J. M. Campistol, J. Buades, T. H. Brannagan, B. J. Kim, J. Oh, Y. Parman, Y. Sekijima, P. N. Hawkins, S. D. Solomon, M. Polydefkis, P. J. Dyck, P. J. Gandhi, S. Goyal, J. Chen, A. L. Strahs, S. V. Nochur, M. T. Sweetser, P. P. Garg, A. K. Vaishnaw, J. A. Gollob, O. B. Suhr, Patisiran, an RNAi Therapeutic, for Hereditary Transthyretin Amyloidosis. N. Engl. J. Med. 379, 11-21 (2018).
17. Brown, K. M., Nair, J. K., Janas, M. M., Anglero-Rodriguez, Y. I., Dang, L. T., Peng, H., . . . & Jadhav, V. (2022). Expanding RNAi therapeutics to extrabepatie tissues with lipophilic conjugates. Nature Biotechnology, 1-9.Kotowska-Zimmer, A., Pewinska, M., & Olejniczak, M. (2021). Artificial miRNAs as therapeutic tools: Challenges and opportunities. Wiley Interdisciplinary Reviews: RNA, 12 (4), e1640.
18. Caron, N. S., Southwell, A. L., Brouwers, C. C., Cengio, L. D., Xie, Y., Black, H. F., . . . & Hayden, M. R. (2020). Potent and sustained huntingtin lowering via AAV5 encoding miRNA preserves striatal volume and cognitive function in a humanized mouse model of Huntington disease. Nucleic acids research, 48 (1), 36-54.
19. Borel, F., Gernoux, G., Sun, H., Stock, R., Blackwood, M., Brown Jr, R. H., & Mueller, C. (2018). Safe and effective superoxide dismutase 1 silencing using artificial microRNA in macaques. Science Translational Medicine, 10 (465), eaau6414.
20. Zlatev, I., Castoreno, A., Brown, C. R., Qin, J., Waldron, S., Schlegel, M. K., . . . & Jadhav, V. (2018). Reversal of siRNA-mediated gene silencing in vivo. Nature biotechnology, 36 (6), 509-511.
21. Krützfeldt, J., Rajewsky, N., Braich, R., Rajeev, K. G., Tuschl, T., Manoharan, M., & Stoffel, M. (2005). Silencing of microRNAs in vivo with ‘antagomirs’. nature, 438 (7068), 685-689.
22. Janssen, H. L., Reesink, H. W., Lawitz, E. J., Zeuzem, S., Rodriguez-Torres, M., Patel, K., . . . & Hodges, M. R. (2013). Treatment of HCV infection by targeting microRNA. New England Journal of Medicine, 368 (18), 1685-1694.
23. Xie, Jun, et al. “MicroRNA-regulated, systemically delivered rAAV9: a step closer to CNS-restricted transgene expression.” Molecular therapy 19.3 (2011): 526-535.
24. Hordeaux, J., Buza, E. L., Jeffrey, B., Song, C., Jahan, T., Yuan, Y., . . . & Wilson, J. M. (2020). MicroRNA-mediated inhibition of transgene expression reduces dorsal root ganglion toxicity by AAV vectors in primates. Science Translational Medicine, 12 (569).
25. Brown, B. D., Venneri, M. A., Zingale, A., Sergi, L. S., & Naldini, L. (2006). Endogenous microRNA regulation suppresses transgene expression in hematopoietic lineages and enables stable gene transfer. Nature medicine, 12 (5), 585-591.
26. Mueller, C., Tang, Q., Gruntman, A., Blomenkamp, K., Teckman, J., Song, L., . . . & Flotte, T. R. (2012). Sustained miRNA-mediated knockdown of mutant AAT with simultaneous augmentation of wild-type AAT has minimal effect on global liver miRNA profiles. Molecular Therapy, 20 (3), 590-600.
27. Beisel, C. L., Chen, Y. Y., Culler, S. J., Hoff, K. G., & Smolke, C. D. (2011). Design of small molecule-responsive microRNAs based on structural requirements for Drosha processing. Nucleic acids research, 39 (7), 2981-2994.
28. Beisel, C. L., Bloom, R. J., & Smolke, C. D. (2014). Construction of ligand-responsive MicroRNAs that operate through inhibition of Drosha processing. In Artificial Riboswitches (pp. 259-267). Humana Press, Totowa, NJ.
29. Xie, J., Tai, P. W., Brown, A., Gong, S., Zhu, S., Wang, Y., . . . & Gao, G. (2020). Effective and accurate gene silencing by a recombinant AAV-compatible microRNA scaffold. Molecular Therapy, 28 (2), 422-430.
30. Xie, J., Mao, Q., Tai, P. W., He, R., Ai, J., Su, Q., . . . & Gao, G. (2017). Short DNA hairpins compromise recombinant adeno-associated virus genome homogeneity. Molecular Therapy, 25 (6), 1363-1374.
31. Fellmann, C., Hoffmann, T., Sridhar, V., Hopfgartner, B., Muhar, M., Roth, M., . . . & Zuber, J. (2013). An optimized microRNA backbone for effective single-copy RNAi. Cell reports, 5 (6), 1704-1713.
32. McBride, J. L., Boudreau, R. L., Harper, S. Q., Staber, P. D., Monteys, A. M., Martins, I., . . . & Davidson, B. L. (2008). Artificial miRNAs mitigate shRNA-mediated toxicity in the brain: implications for the therapeutic development of RNAi. Proceedings of the National Academy of Sciences, 105 (15), 5868-5873
33. Ausländer, S., & Fussenegger, M. (2017). Synthetic RNA-based switches for mammalian gene expression control. Current opinion in biotechnology, 48, 54-60.
34. Zhong, G., Wang, H., He, W., Li, Y., Mou, H., Tickner, Z. J., . . . & Farzan, M. (2020). A reversible RNA on-switch that controls gene expression of AAV-delivered therapeutics in vivo. Nature biotechnology, 38 (2), 169-175.
35. Monteys, A. M., Hundley, A. A., Ranum, P. T., Tecedor, L., Muehlmatt, A., Lim, E., . . . & Davidson, B. L. (2021). Regulated control of gene therapies by drug-induced splicing. Nature, 596 (7871), 291-295.
36. Aronesty. E. (2011) ea-utils: “Command-line tools for processing biological sequencing data”.
37. Dobin, A., Davis, C. A., Schlesinger, F., Drenkow, J., Zaleski, C., Jha, S., Batut, P., Chaisson, M. and Gingeras, T. R. (2013) STAR: ultrafast universal RNA-seq aligner. Bioinformatics, 29, 15-21.
38. Liao, Y., Smyth, G. K. and Shi, W. (2014) featureCounts: an efficient general purpose program for assigning sequence reads to genomic features. Bioinformatics, 30, 923-930.
39. Love, M. I., Huber, W. and Anders, S. (2014) Moderated estimation of fold change and dispersion for RNA-seq data with DESeq2. Genome Biology, 15, 550.
40. Fong, S., Yates, B., Sihn, C. R., Mattis, A. N., Mitchell, N., Liu, S., . . . & Wong, W. Y. (2022). Interindividual variability in transgene mRNA and protein production following adeno-associated virus gene therapy for hemophilia A. Nature medicine, 28 (4), 789-797.
41. Rangarajan, S., Walsh, L., Lester, W., Perry, D., Madan, B., Laffan, M., . . . & Pasi, K. J. (2017). AAV5-factor VIII gene transfer in severe hemophilia A. New England Journal of Medicine, 377 (26), 2519-2530.
42. Tang, F., Wong, H., & Ng, C. M. (2021). Rational Clinical Dose Selection of Adeno-Associated Virus-Mediated Gene Therapy Based on Allometric Principles. Clinical Pharmacology & Therapeutics, 110 (3), 803-807.
43. Hu. B., Zhong, L., Weng, Y., Peng, L., Huang, Y., Zhao, Y., & Liang, X. J. (2020). Therapeutic siRNA: state of the art. Signal transduction and targeted therapy, 5 (1), 1-25.
44. Hansel, T. T., Kropshofer, H., Singer, T., Mitchell, J. A., & George, A. J. (2010). The safety and side effects of monoclonal antibodies. Nature reviews Drug discovery, 9 (4), 325-338.
45. Kaczmarek, R., & Herzog, R. W. (2022). Treatment-induced hemophilic thrombosis. Mol Ther, 30 (2), 505-506.
46. Verdera, H. C., Kuranda, K., & Mingozzi, F. (2020). AAV vector immunogenicity in humans: a long journey to successful gene transfer. Molecular Therapy, 28 (3), 723-746.
47. Mueller, C., Tang, Q., Gruntman, A., Blomenkamp, K., Teckman, J., Song, L., . . . & Flotte, T. R. (2012). Sustained miRNA-mediated knockdown of mutant AAT with simultaneous augmentation of wild-type AAT has minimal effect on global liver miRNA profiles. Molecular Therapy, 20 (3), 590-600.
48. Schlegel, M. K., Matsuda, S., Brown, C. R., Harp, J. M., Barry, J. D., Berman, D., . . . & Maier, M. A. (2021). Overcoming GNA/RNA base-pairing limitations using isonucleotides improves the pharmacodynamic activity of ESC+ GalNAc-siRNAs. Nucleic acids research, 49 (19), 10851-10867.

1. A universal double stranded ribonucleic acid (dsRNA) agent, comprising a sense strand and an antisense strand forming a double stranded region,
(a) wherein the antisense strand comprises at least 15 contiguous nucleotides differing by no more than 3 nucleotides from any one of the antisense strand nucleotide sequences in Table 2;
(b) wherein the sense strand comprises at least 15 contiguous nucleotides differing by no more than 3 nucleotides from any one of the sense strand nucleotide sequences in Table 2 and the antisense strand comprises at least 15 contiguous nucleotides differing by no more than 3 nucleotides from any one of the antisense strand nucleotide sequences in Table 2; or
(c) wherein the antisense strand comprises a region of complementarity to any one of the target nucleotide sequences in Table 3.

2.-5. (canceled)

6. The universal dsRNA agent of claim 1, wherein all of the nucleotides of the sense strand comprise a nucleotide modification are modified nucleotides; all of the nucleotides of the antisense strand comprise a nucleotide modification are modified nucleotides; or all of the nucleotides of the sense strand and all of the nucleotides of the antisense strand comprise a nucleotide modification are modified nucleotides.

7. The universal dsRNA agent of claim 6, wherein at least one of the nucleotide modifications is selected from the group consisting of a deoxy-nucleotide modification, a 3′-terminal deoxythimidine (dT) nucleotide modification, a 2′-O-methyl nucleotide modification, a 2′-fluoro nucleotide modification, a 2′-deoxy-nucleotide modification, a locked nucleotide modification, an unlocked nucleotide modification, a conformationally restricted nucleotide modification, a constrained ethyl nucleotide modification, an abasic nucleotide modification, a 2′-amino-nucleotide modification, a 2′-O-allyl-nucleotide modification, 2′-C-alkyl-nucleotide modification, 2′-hydroxly-nucleotide modification, a 2′-methoxyethyl-nucleotide modification, a 2′-O-alkyl-nucleotide modification, a morpholino nucleotide modification, a phosphoramidate modification, a non-natural base comprising nucleotide modification, a tetrahydropyran nucleotide modification, a 1,5-anhydrohexitol nucleotide modification, a cyclohexenyl nucleotide modification, a nucleotide comprising a 5′-phosphate modification, a nucleotide comprising a 5′-phosphate mimic modification, a thermally destabilizing nucleotide modification, a glycol nucleotide (GNA) modification, a nucleotide comprising a 2′ phosphate modification, and a 2-O—(N-methylacetamide) nucleotide modification; and combinations thereof.

8.-16. (canceled)

17. The universal dsRNA agent of claim 1, wherein each strand is independently no more than 30 nucleotides in length.

18.-21. (canceled)

22. The universal dsRNA agent of claim 1, further comprising a ligand.

23.-28. (canceled)

29. The universal dsRNA agent of claim 1, wherein the dsRNA agent further comprises at least one phosphorothioate or methylphosphonate internucleotide linkage.

30.-38. (canceled)

39. A cell containing the universal dsRNA agent of claim 1.

40. A vector comprising the universal dsRNA agent of claim 1.

41.-46. (canceled)

47. A pharmaceutical composition comprising the universal dsRNA agent of claim 1 and a pharmaceutically acceptable carrier.

48.-53. (canceled)

54. A REVERSIR compound, comprising a single stranded oligonucleotide 6-30 nucleotides in length and comprising a nucleotide sequence which is at least about 90% complementary to any one of the antisense strand nucleotide sequences in Table 2 or Table 3.

55.-57. (canceled)

58. The REVERSIR compound of claim 54, wherein all of the nucleotides of the oligonucleotide comprise a nucleotide modification are modified nucleotides.

59. The REVERSIR compound of claim 54, wherein at least one of the nucleotide modifications is a nucleobase modification and/or a sugar modification.

60.-62. (canceled)

63. The REVERSIR compound of claim 54, further comprising a ligand.

64.-69. (canceled)

70. The REVERSIR compound of claim 54, wherein the oligonucleotide further comprises at least one phosphorothioate or methylphosphonate internucleotide linkage.

71. The REVERSIR compound of claim 54, wherein the oligonucleotide is 6-15, 7-11, 8-10, 15-25, 17-25, 19-25, or 21-25 nucleotides in length.

72. (canceled)

73. A cell containing the REVERSIR compound of claim 54.

74. A system for on-demand expression of a transgene, the system comprising
(a) an expression vector encoding a transgene and comprising a universal iRNA target site;
the universal dsRNA agent of claim 1, which recognizes and binds to the universal iRNA target site to thereby inhibit the expression of the transgene; and, optionally,
a REVERSIR compound that abrogates the iRNA activity of the universal dsRNA agent to thereby allow the expression of the transgene; or
(b) an expression vector encoding a transgene and a double stranded ribonucleic acid (dsRNA) agent targeting the transgene; wherein expression of the transgene is inhibited by the expression of the dsRNA agent targeting the transgene; and, optionally, a REVERSIR compound that abrogates the iRNA activity of the dsRNA agent to thereby allow the expression of the transgene.

75.-114. (canceled)

115. The system of claim 74, wherein the REVERSIR compound comprises a single stranded oligonucleotide 6-30 nucleotides in length and comprising a nucleotide sequence which is at least about 90% complementary to any one of the antisense strand nucleotide sequences in any one of Table 2 or Table 3.

116.-136. (canceled)

137. A method of modulating expression of a transgene in a cell, the method comprising
contacting the cell with an expression vector encoding a transgene and comprising a universal iRNA target site;
contacting the cell with the universal dsRNA agent of claim 1 which recognizes and binds to the universal iRNA target site and inhibits the expression of the transgene; and, optionally,
further contacting the cell with a REVERSIR compound that abrogates the iRNA activity of the universal dsRNA agent, thereby allowing expression of the transgene.

138. (canceled)

139. A method of treating a subject in need thereof, the method comprising
(a) contacting an expression vector encoding a therapeutic transgene and comprising a universal iRNA target site administered to the subject with a universal double stranded ribonucleic acid (dsRNA) agent which recognizes and binds to the universal iRNA target site to thereby inhibit expression of the transgene, thereby treating the subject;
(b) contacting an expression vector encoding a therapeutic transgene and a double stranded ribonucleic acid (dsRNA) agent targeting the transgene administered to the subject with a REVERSIR compound that abrogates the iRNA activity of the dsRNA agent to thereby allow expression of the transgene, thereby treating the subject; or
(c) administering to said subject an expression vector encoding a transgene and comprising a universal iRNA target site;
allowing expression of the transgene until a desired level of expression has been achieved; and once a desired level of expression of the transgene has been achieved, administering to said subject a universal double stranded ribonucleic acid (dsRNA) agent, comprising a sense strand and an antisense strand forming a double stranded region, which recognizes and binds to the universal iRNA target site to thereby inhibit the expression of the transgene, thereby treating said subject.

140.-204. (canceled)
